SEQUENCE LISTING (167 sequences)

SEQ ID NO: 1 - protein, 16 aa, synthetic construct
  REGION 1..16: note = Description of Artificial Sequence: Synthetic peptide
RSSKSLLHRNGITYLY

SEQ ID NO: 2 - protein, 7 aa, synthetic construct
  REGION 1..7: note = Description of Artificial Sequence: Synthetic peptide
EMSNLAS

SEQ ID NO: 3 - protein, 9 aa, synthetic construct
  REGION 1..9: note = Description of Artificial Sequence: Synthetic peptide
GQNLEFPLT

SEQ ID NO: 4 - protein, 11 aa, synthetic construct
  REGION 1..11: note = Description of Artificial Sequence: Synthetic peptide
KASQNVGTAVA

SEQ ID NO: 5 - protein, 7 aa, synthetic construct
  REGION 1..7: note = Description of Artificial Sequence: Synthetic peptide
SVSNRYT

SEQ ID NO: 6 - protein, 9 aa, synthetic construct
  REGION 1..9: note = Description of Artificial Sequence: Synthetic peptide
QQYSSYPFT

SEQ ID NO: 7 - protein, 11 aa, synthetic construct
  REGION 1..11: note = Description of Artificial Sequence: Synthetic peptide
HASQGINNNIG

SEQ ID NO: 8 - protein, 7 aa, synthetic construct
  REGION 1..7: note = Description of Artificial Sequence: Synthetic peptide
HKSNLED

SEQ ID NO: 9 - protein, 9 aa, synthetic construct
  REGION 1..9: note = Description of Artificial Sequence: Synthetic peptide
VQYAQFPYT

SEQ ID NO: 10 - protein, 16 aa, synthetic construct
  REGION 1..16: note = Description of Artificial Sequence: Synthetic peptide
RSSKSLLHRNGITYLY

SEQ ID NO: 11 - protein, 7 aa, synthetic construct
  REGION 1..7: note = Description of Artificial Sequence: Synthetic peptide
QMSNLAS

SEQ ID NO: 12 - protein, 9 aa, synthetic construct
  REGION 1..9: note = Description of Artificial Sequence: Synthetic peptide
AQNLELPLT

SEQ ID NO: 13 - protein, 17 aa, synthetic construct
  REGION 1..17: note = Description of Artificial Sequence: Synthetic peptide
KSSQSLLYSSNQKNYLA

SEQ ID NO: 14 - protein, 7 aa, synthetic construct
  REGION 1..7: note = Description of Artificial Sequence: Synthetic peptide
WASTRES

SEQ ID NO: 15 - protein, 9 aa, synthetic construct
  REGION 1..9: note = Description of Artificial Sequence: Synthetic peptide
HQYYSSPLT

SEQ ID NO: 16 - protein, 11 aa, synthetic construct
  REGION 1..11: note = Description of Artificial Sequence: Synthetic peptide
RASQEISGYLS

SEQ ID NO: 17 - protein, 7 aa, synthetic construct
  REGION 1..7: note = Description of Artificial Sequence: Synthetic peptide
AASTLDS

SEQ ID NO: 18 - protein, 9 aa, synthetic construct
  REGION 1..9: note = Description of Artificial Sequence: Synthetic peptide
LQYASYPLT

SEQ ID NO: 19 - protein, 10 aa, synthetic construct
  REGION 1..10: note = Description of Artificial Sequence: Synthetic peptide
SANSSVSFMH

SEQ ID NO: 20 - protein, 7 aa, synthetic construct
  REGION 1..7: note = Description of Artificial Sequence: Synthetic peptide
STSSLAS

SEQ ID NO: 21 - protein, 9 aa, synthetic construct
  REGION 1..9: note = Description of Artificial Sequence: Synthetic peptide
QQRSSYPLT

SEQ ID NO: 22 - protein, 11 aa, synthetic construct
  REGION 1..11: note = Description of Artificial Sequence: Synthetic peptide
KASQNVVTYVA

SEQ ID NO: 23 - protein, 7 aa, synthetic construct
  REGION 1..7: note = Description of Artificial Sequence: Synthetic peptide
SASYRYS

SEQ ID NO: 24 - protein, 9 aa, synthetic construct
  REGION 1..9: note = Description of Artificial Sequence: Synthetic peptide
QQYHSYPYT

SEQ ID NO: 25 - protein, 15 aa, synthetic construct
  REGION 1..15: note = Description of Artificial Sequence: Synthetic peptide
RASESVDIYGISFLH

SEQ ID NO: 26 - protein, 7 aa, synthetic construct
  REGION 1..7: note = Description of Artificial Sequence: Synthetic peptide
RASNLDS

SEQ ID NO: 27 - protein, 9 aa, synthetic construct
  REGION 1..9: note = Description of Artificial Sequence: Synthetic peptide
QQSNKDPLT

SEQ ID NO: 28 - protein, 17 aa, synthetic construct
  REGION 1..17: note = Description of Artificial Sequence: Synthetic peptide
KSSQSLLYSSNQKIYLA

SEQ ID NO: 29 - protein, 9 aa, synthetic construct
  REGION 1..9: note = Description of Artificial Sequence: Synthetic peptide
QQYYNSPLT

SEQ ID NO: 30 - protein, 12 aa, synthetic construct
  REGION 1..12: note = Description of Artificial Sequence: Synthetic peptide
SASSSISSDYLH

SEQ ID NO: 31 - protein, 7 aa, synthetic construct
  REGION 1..7: note = Description of Artificial Sequence: Synthetic peptide
RTSNLAS

SEQ ID NO: 32 - protein, 9 aa, synthetic construct
  REGION 1..9: note = Description of Artificial Sequence: Synthetic peptide
QQGTSLPRA

SEQ ID NO: 33 - protein, 17 aa, synthetic construct
  REGION 1..17: note = Description of Artificial Sequence: Synthetic peptide
KSSQSLLHSGNQKNYMT

SEQ ID NO: 34 - protein, 9 aa, synthetic construct
  REGION 1..9: note = Description of Artificial Sequence: Synthetic peptide
QNDYSYPLT

SEQ ID NO: 35 - protein, 7 aa, synthetic construct
  REGION 1..7: note = Description of Artificial Sequence: Synthetic peptide
VASTLDS

SEQ ID NO: 36 - protein, 9 aa, synthetic construct
  REGION 1..9: note = Description of Artificial Sequence: Synthetic peptide
LQYANYPYT

SEQ ID NO: 37 - protein, 11 aa, synthetic construct
  REGION 1..11: note = Description of Artificial Sequence: Synthetic peptide
SASQDIINYLN

SEQ ID NO: 38 - protein, 7 aa, synthetic construct
  REGION 1..7: note = Description of Artificial Sequence: Synthetic peptide
STSSLHS

SEQ ID NO: 39 - protein, 9 aa, synthetic construct
  REGION 1..9: note = Description of Artificial Sequence: Synthetic peptide
HQYSQLPYT

SEQ ID NO: 40 - protein, 11 aa, synthetic construct
  REGION 1..11: note = Description of Artificial Sequence: Synthetic peptide
SASQDIFNYLN

SEQ ID NO: 41 - protein, 7 aa, synthetic construct
  REGION 1..7: note = Description of Artificial Sequence: Synthetic peptide
YTSSLHS

SEQ ID NO: 42 - protein, 9 aa, synthetic construct
  REGION 1..9: note = Description of Artificial Sequence: Synthetic peptide
QQYSNLPYT

SEQ ID NO: 43 - protein, 10 aa, synthetic construct
  REGION 1..10: note = Description of Artificial Sequence: Synthetic peptide
GYTFTDYYVN

SEQ ID NO: 44 - protein, 17 aa, synthetic construct
  REGION 1..17: note = Description of Artificial Sequence: Synthetic peptide
DIHPNSGDTTYNQKFKD

SEQ ID NO: 45 - protein, 10 aa, synthetic construct
  REGION 1..10: note = Description of Artificial Sequence: Synthetic peptide
RRYDYDGFAY

SEQ ID NO: 46 - protein, 17 aa, synthetic construct
  REGION 1..17: note = Description of Artificial Sequence: Synthetic peptide
DIHPNNGGITYNQKFKG

SEQ ID NO: 47 - protein, 10 aa, synthetic construct
  REGION 1..10: note = Description of Artificial Sequence: Synthetic peptide
GFTFSDYGMH

SEQ ID NO: 48 - protein, 17 aa, synthetic construct
  REGION 1..17: note = Description of Artificial Sequence: Synthetic peptide
YINSDSNTIYYADTVKG

SEQ ID NO: 49 - protein, 6 aa, synthetic construct
  REGION 1..6: note = Description of Artificial Sequence: Synthetic peptide
LVAPDY

SEQ ID NO: 50 - protein, 10 aa, synthetic construct
  REGION 1..10: note = Description of Artificial Sequence: Synthetic peptide
GHTFTSNWIH

SEQ ID NO: 51 - protein, 17 aa, synthetic construct
  REGION 1..17: note = Description of Artificial Sequence: Synthetic peptide
EIDPSDSYTYYNQKFKG

SEQ ID NO: 52 - protein, 9 aa, synthetic construct
  REGION 1..9: note = Description of Artificial Sequence: Synthetic peptide
PGRNSNFAY

SEQ ID NO: 53 - protein, 12 aa, synthetic construct
  REGION 1..12: note = Description of Artificial Sequence: Synthetic peptide
GFSLSTSGMGVT

SEQ ID NO: 54 - protein, 16 aa, synthetic construct
  REGION 1..16: note = Description of Artificial Sequence: Synthetic peptide
HIFWDGDKRYNPSLKS

SEQ ID NO: 55 - protein, 12 aa, synthetic construct
  REGION 1..12: note = Description of Artificial Sequence: Synthetic peptide
YYYFDYGYAIDY

SEQ ID NO: 56 - protein, 10 aa, synthetic construct
  REGION 1..10: note = Description of Artificial Sequence: Synthetic peptide
GYTFTSYVIH

SEQ ID NO: 57 - protein, 17 aa, synthetic construct
  REGION 1..17: note = Description of Artificial Sequence: Synthetic peptide
YIDPSNDDTKYNEKFKG

SEQ ID NO: 58 - protein, 12 aa, synthetic construct
  REGION 1..12: note = Description of Artificial Sequence: Synthetic peptide
EAYYGGLYGMDY

SEQ ID NO: 59 - protein, 10 aa, synthetic construct
  REGION 1..10: note = Description of Artificial Sequence: Synthetic peptide
GYTFIDYTIH

SEQ ID NO: 60 - protein, 18 aa, synthetic construct
  REGION 1..18: note = Description of Artificial Sequence: Synthetic peptide
WIFPGSTNDTKYNDKFKG

SEQ ID NO: 61 - protein, 7 aa, synthetic construct
  REGION 1..7: note = Description of Artificial Sequence: Synthetic peptide
YRTDFDY

SEQ ID NO: 62 - protein, 10 aa, synthetic construct
  REGION 1..10: note = Description of Artificial Sequence: Synthetic peptide
GYSFTSYWMH

SEQ ID NO: 63 - protein, 17 aa, synthetic construct
  REGION 1..17: note = Description of Artificial Sequence: Synthetic peptide
DIDPSNSYAYHSQKFKG

SEQ ID NO: 64 - protein, 11 aa, synthetic construct
  REGION 1..11: note = Description of Artificial Sequence: Synthetic peptide
ADGTSHWYFDV

SEQ ID NO: 65 - protein, 10 aa, synthetic construct
  REGION 1..10: note = Description of Artificial Sequence: Synthetic peptide
GYTFTDYYLN

SEQ ID NO: 66 - protein, 17 aa, synthetic construct
  REGION 1..17: note = Description of Artificial Sequence: Synthetic peptide
WIYPGSSDTKHNENFKG

SEQ ID NO: 67 - protein, 8 aa, synthetic construct
  REGION 1..8: note = Description of Artificial Sequence: Synthetic peptide
YSNFFFDY

SEQ ID NO: 68 - protein, 12 aa, synthetic construct
  REGION 1..12: note = Description of Artificial Sequence: Synthetic peptide
GFSLSTSGMGVS

SEQ ID NO: 69 - protein, 16 aa, synthetic construct
  REGION 1..16: note = Description of Artificial Sequence: Synthetic peptide
HIYWDDDKRYNPSLKS

SEQ ID NO: 70 - protein, 11 aa, synthetic construct
  REGION 1..11: note = Description of Artificial Sequence: Synthetic peptide
SSQGLYSSYDY

SEQ ID NO: 71 - protein, 10 aa, synthetic construct
  REGION 1..10: note = Description of Artificial Sequence: Synthetic peptide
GYTFTSYVMH

SEQ ID NO: 72 - protein, 17 aa, synthetic construct
  REGION 1..17: note = Description of Artificial Sequence: Synthetic peptide
YIDPDNDGTKYNEKFKG

SEQ ID NO: 73 - protein, 10 aa, synthetic construct
  REGION 1..10: note = Description of Artificial Sequence: Synthetic peptide
GFTFSDYYMS

SEQ ID NO: 74 - protein, 17 aa, synthetic construct
  REGION 1..17: note = Description of Artificial Sequence: Synthetic peptide
YISSGGGSSYYPDSVKG

SEQ ID NO: 75 - protein, 9 aa, synthetic construct
  REGION 1..9: note = Description of Artificial Sequence: Synthetic peptide
LPHYFAMDC

SEQ ID NO: 76 - protein, 17 aa, synthetic construct
  REGION 1..17: note = Description of Artificial Sequence: Synthetic peptide
YISSGGGSKYYPDLVKG

SEQ ID NO: 77 - protein, 9 aa, synthetic construct
  REGION 1..9: note = Description of Artificial Sequence: Synthetic peptide
LPHYFAMDY

SEQ ID NO: 78 - protein, 17 aa, synthetic construct
  REGION 1..17: note = Description of Artificial Sequence: Synthetic peptide
YISSGGGSSYYPDAVKG

SEQ ID NO: 79 - protein, 17 aa, synthetic construct
  REGION 1..17: note = Description of Artificial Sequence: Synthetic peptide
YISSGGGSSYYPDLVKG

SEQ ID NO: 80 - protein, 10 aa, synthetic construct
  REGION 1..10: note = Description of Artificial Sequence: Synthetic peptide
GFTFSDYYMA

SEQ ID NO: 81 - protein, 17 aa, synthetic construct
  REGION 1..17: note = Description of Artificial Sequence: Synthetic peptide
NINYDGFNTYYLDSLKS

SEQ ID NO: 82 - protein, 10 aa, synthetic construct
  REGION 1..10: note = Description of Artificial Sequence: Synthetic peptide
GGYWSLYFDY

SEQ ID NO: 83 - protein, 10 aa, synthetic construct
  REGION 1..10: note = Description of Artificial Sequence: Synthetic peptide
GYTFTDYYIN

SEQ ID NO: 84 - protein, 17 aa, synthetic construct
  REGION 1..17: note = Description of Artificial Sequence: Synthetic peptide
WIYPGGGHTKYNEKFKG

SEQ ID NO: 85 - protein, 8 aa, synthetic construct
  REGION 1..8: note = Description of Artificial Sequence: Synthetic peptide
YSNYYFDF

SEQ ID NO: 86 - protein, 10 aa, synthetic construct
  REGION 1..10: note = Description of Artificial Sequence: Synthetic peptide
GYTFTSYYIQ

SEQ ID NO: 87 - protein, 17 aa, synthetic construct
  REGION 1..17: note = Description of Artificial Sequence: Synthetic peptide
WIYPGDGTTNYNENFKG

SEQ ID NO: 88 - protein, 8 aa, synthetic construct
  REGION 1..8: note = Description of Artificial Sequence: Synthetic peptide
YGLVPFDY

SEQ ID NO: 89 - protein, 10 aa, synthetic construct
  REGION 1..10: note = Description of Artificial Sequence: Synthetic peptide
GNTFNSNYIQ

SEQ ID NO: 90 - protein, 17 aa, synthetic construct
  REGION 1..17: note = Description of Artificial Sequence: Synthetic peptide
WIYPGDGSTNYSEKFKG

SEQ ID NO: 91 - protein, 8 aa, synthetic construct
  REGION 1..8: note = Description of Artificial Sequence: Synthetic peptide
YGPVPFDY

SEQ ID NO: 92 - protein, 112 aa, synthetic construct
  REGION 1..112: note = Description of Artificial Sequence: Synthetic polypeptide
EIVMTQAAFSNPVTLGTSASISCRSSKSLLHRNGITYLYWYLQKPGQSPQLLIYEMSNLASGVPDRFSSSGSGTDFTLRISRVEAEDVGVYYCGQNLEFPLTFGAGTKLELK

SEQ ID NO: 93 - protein, 112 aa, synthetic construct
  REGION 1..112: note = Description of Artificial Sequence: Synthetic polypeptide
EIVMTQAAFSNPVTLGTSASISCRSSKSLLHRNGITYLYWYLQKPGQSPQLLIYEMSNLASGVPDRFSGSGSGTDFTLRISRVEAEDVGVYYCGQNLEFPLTFGAGTKLELK

SEQ ID NO: 94 - protein, 107 aa, synthetic construct
  REGION 1..107: note = Description of Artificial Sequence: Synthetic polypeptide
DIVMTQSQKFMSTTVGDRVSITCKASQNVGTAVAWYQQKPGQSPKLLIYSVSNRYTGVPDRFTGSGSGTDFTLTISNMQSEDLADYFCQQYSSYPFTFGAGTKLELK

SEQ ID NO: 95 - protein, 107 aa, synthetic construct
  REGION 1..107: note = Description of Artificial Sequence: Synthetic polypeptide
DILMTQSPSSMSVSLGDTVSITCHASQGINNNIGWLQQKPGKSFKGLIYHKSNLEDGVPSRFSGSGSGADYSLTISSLESEDFADYYCVQYAQFPYTFGGGTKLEIK

SEQ ID NO: 96 - protein, 112 aa, synthetic construct
  REGION 1..112: note = Description of Artificial Sequence: Synthetic polypeptide
DIVMTQAAFYNPVTLGTSASISCRSSKSLLHRNGITYLYWYLQKPGQSPQLLIYQMSNLASGVPDRFSSSGSGADFTLRISRVEAEDVGVYYCAQNLELPLTFGAGTKLELK

SEQ ID NO: 97 - protein, 113 aa, synthetic construct
  REGION 1..113: note = Description of Artificial Sequence: Synthetic polypeptide
DIVMSQSPSSLAVSVGEKVTMTCKSSQSLLYSSNQKNYLAWYQQKPGQSPKLLIYWASTRESGVPDRFTGSGSGTDFTLTISSVKAEDLAVYYCHQYYSSPLTFGAGTKLELK

SEQ ID NO: 98 - protein, 107 aa, synthetic construct
  REGION 1..107: note = Description of Artificial Sequence: Synthetic polypeptide
DIQMTQSPSSLSASLGERVSLTCRASQEISGYLSWLQQKPDGTIKRLIYAASTLDSGVPKRFSGSRSGSDYSLTISSLESEDFADYYCLQYASYPLTFGAGTKLELK

SEQ ID NO: 99 - protein, 106 aa, synthetic construct
  REGION 1..106: note = Description of Artificial Sequence: Synthetic polypeptide
QIVLTQSPGIMSASPGEKVTITCSANSSVSFMHWFQQKPGTSPKIWIYSTSSLASGVPARFSGSGSGTSYSLTISRMEAEDAATYYCQQRSSYPLTFGAGTKLELK

SEQ ID NO: 100 - protein, 107 aa, synthetic construct
  REGION 1..107: note = Description of Artificial Sequence: Synthetic polypeptide
DIVMTQSQKFLSTSVGDRVRVTCKASQNVVTYVAWYQQKPGQSPKSLIYSASYRYSGVPDRFTGSGSGTYFTLTINNVQFEDLAEYFCQQYHSYPYTFGGGTKLEIK

SEQ ID NO: 101 - protein, 111 aa, synthetic construct
  REGION 1..111: note = Description of Artificial Sequence: Synthetic polypeptide
DIVLTQSPASLAVSLGQRATISCRASESVDIYGISFLHWYQQKPGQPPKHLIYRASNLDSGIPARFSGSGSRTDFTLTINPVETDDVATYYCQQSNKDPLTFGAGTKLELK

SEQ ID NO: 102 - protein, 113 aa, synthetic construct
  REGION 1..113: note = Description of Artificial Sequence: Synthetic polypeptide
DIVMSQSPSSLTVSVGEKVTMSCKSSQSLLYSSNQKIYLAWFQQKPGQSPKLLIYWASTRESGVPDRFTGSGSGTDFTLTISSVKAEDLAVYYCQQYYNSPLTFGAGTKLELK

SEQ ID NO: 103 - protein, 108 aa, synthetic construct
  REGION 1..108: note = Description of Artificial Sequence: Synthetic polypeptide
EIVLTQSPTTMAASPGEKITITCSASSSISSDYLHWYQQKPGFSPELLIYRTSNLASGVPARFSGSGSGTSYSLTIGSMEAEDVATYYCQQGTSLPRAFGGGTKLEIK

SEQ ID NO: 104 - protein, 113 aa, synthetic construct
  REGION 1..113: note = Description of Artificial Sequence: Synthetic polypeptide
DIVMTQSPSSLTVTAGEKVTMSCKSSQSLLHSGNQKNYMTWYQQKPGQPPKLLIYWASTRESGVPDRFTGSGSGTDFTLTISSVQAEDLAVYYCQNDYSYPLTFGAGTKLELK

SEQ ID NO: 105 - protein, 107 aa, synthetic construct
  REGION 1..107: note = Description of Artificial Sequence: Synthetic polypeptide
DIQMTQSPSSLSASPGERVSLTCRASQEISGYLSWLQQKPDGTIKRLIYVASTLDSGVPKRFSGSRSGSDYSLTISSLESEDFADYYCLQYANYPYTFGGGTKLEIK

SEQ ID NO: 106 - protein, 107 aa, synthetic construct
  REGION 1..107: note = Description of Artificial Sequence: Synthetic polypeptide
DIQMTQTTSSLSASLGDRVTISCSASQDIINYLNWYQQKPDGTVKLLIYSTSSLHSGVSLRFSGSGSGTDYSLTISNLEPEDVATYYCHQYSQLPYTFGGGTKLEIK

SEQ ID NO: 107 - protein, 107 aa, synthetic construct
  REGION 1..107: note = Description of Artificial Sequence: Synthetic polypeptide
DIQMTQTTSSLSASLGDRVTISCSASQDIFNYLNWYQQKPDGTVKLLIYYTSSLHSGVPSRFSGSGSGTDFSLTISNLEPEDIATYYCQQYSNLPYTFGGGTKLEIK

SEQ ID NO: 108 - protein, 119 aa, synthetic construct
  REGION 1..119: note = Description of Artificial Sequence: Synthetic polypeptide
EVQLQQSGPELVKPGSSVKISCKASGYTFTDYYVNWVKQSHGKSLEWFGDIHPNSGDTTYNQKFKDKATLTVDKSSSTAYMELRSLTSEDSAVYYCARRRYDYDGFAYWGQGTLVTVSA

SEQ ID NO: 109 - protein, 119 aa, synthetic construct
  REGION 1..119: note = Description of Artificial Sequence: Synthetic polypeptide
EVQLQQSGPELVKPGASVKISCKASGYTFTDYYVNWVKQSHGKSLEWFGDIHPNNGGITYNQKFKGKASLTVDKSSSTAYMELRSLTSEDSAVYYCARRRYDYDGFAYWGQGTLVTVSA

SEQ ID NO: 110 - protein, 115 aa, synthetic construct
  REGION 1..115: note = Description of Artificial Sequence: Synthetic polypeptide
EVQLVESGGGLVKPGGSLKLSCAASGFTFSDYGMHWVRQTPEKGLEWIAYINSDSNTIYYADTVKGRFTISRDNARKTLYLQMTSLRSEDTAMYYCSPLVAPDYWGQGTTLTVSS

SEQ ID NO: 111 - protein, 118 aa, synthetic construct
  REGION 1..118: note = Description of Artificial Sequence: Synthetic polypeptide
QVQLQQPGAELVKPGASVKLSCKASGHTFTSNWIHWVKQRPGQGLEWIGEIDPSDSYTYYNQKFKGKATLTVDKSSSTAYMQLSSLTSEDSAVYSCACPGRNSNFAYWGQGTTLTVSS

SEQ ID NO: 112 - protein, 122 aa, synthetic construct
  REGION 1..122: note = Description of Artificial Sequence: Synthetic polypeptide
QVTLKESGPGILQPSQTLSLTCSFSGFSLSTSGMGVTWIRKPSGQGLEWLAHIFWDGDKRYNPSLKSRLTISKDSSSNQVFLMITGVGTADAATYYCARYYYFDYGYAIDYWGQGTSVTVSS

SEQ ID NO: 113 - protein, 121 aa, synthetic construct
  REGION 1..121: note = Description of Artificial Sequence: Synthetic polypeptide
EVQLQQSGPELVKPGASVKMSCKASGYTFTSYVIHWVKQKPGQGLEWIGYIDPSNDDTKYNEKFKGKATLTSDKSSSTAYMELSSLTSEDSAVYYCAREAYYGGLYGMDYWGQGTSVTVSS

SEQ ID NO: 114 - protein, 117 aa, synthetic construct
  REGION 1..117: note = Description of Artificial Sequence: Synthetic polypeptide
QVQLQQSGPELVKPGASVKLSCKASGYTFIDYTIHWVKQSPGQGLEWIGWIFPGSTNDTKYNDKFKGKATMTADKSSSTAYMQLSSLTSEDSAVYFCARYRTDFDYWGQGTTLTVSS

SEQ ID NO: 115 - protein, 120 aa, synthetic construct
  REGION 1..120: note = Description of Artificial Sequence: Synthetic polypeptide
QVQLQQPGAELVKPGASVKLSCKASGYSFTSYWMHWVRQRPGQGLEWIGDIDPSNSYAYHSQKFKGKATLTVDKSSSTAYMQLSSLTSEDSAVYFCARADGTSHWYFDVWGAGTAVTVSS

SEQ ID NO: 116 - protein, 117 aa, synthetic construct
  REGION 1..117: note = Description of Artificial Sequence: Synthetic polypeptide
QIQLQQSGPELVKPGASVKISCKASGYTFTDYYLNWVKQRPGHGLEWIGWIYPGSSDTKHNENFKGKATLTVDTYSSTAYMQLGSLTSEDSAVYFCTRYSNFFFDYWGQGTTLTVSS

SEQ ID NO: 117 - protein, 121 aa, synthetic construct
  REGION 1..121: note = Description of Artificial Sequence: Synthetic polypeptide
QVTLKESGPGILQPSQTLSLTCSFSGFSLSTSGMGVSWIRQPSGKGLEWLTHIYWDDDKRYNPSLKSRLTISKDTSRNQVFLEITSVDTADTATYFCARSSQGLYSSYDYWGQGTTLTVSS

SEQ ID NO: 118 - protein, 121 aa, synthetic construct
  REGION 1..121: note = Description of Artificial Sequence: Synthetic polypeptide
EVQLQQSGPELVKPGASVKMSCKASGYTFTSYVMHWVRQKPGQGLEWIGYIDPDNDGTKYNEKFKGKATLTSDKSSSTAYMELSSLTSEDSAVYYCAREAYYGGLYGMDYWGQGSSVTVSS

SEQ ID NO: 119 - protein, 118 aa, synthetic construct
  REGION 1..118: note = Description of Artificial Sequence: Synthetic polypeptide
EVQLVESGGGLVQPGGSLKLSCAASGFTFSDYYMSWVRQTPEKRLEWVAYISSGGGSSYYPDSVKGRFTISRDNTKNTLYLQMSSLKSEDTAVYYCARLPHYFAMDCWGQGTSVTVSS

SEQ ID NO: 120 - protein, 119 aa, synthetic construct
  REGION 1..119: note = Description of Artificial Sequence: Synthetic polypeptide
EVKLVESEGGLVQPGSSMKLSCTASGFTFSDYYMAWVRQVPEKGLEWVANINYDGFNTYYLDSLKSRFIISRDNAKNILYLQMSSLKSEDTATYYCARGGYWSLYFDYWGQGTTLTVSS

SEQ ID NO: 121 - protein, 117 aa, synthetic construct
  REGION 1..117: note = Description of Artificial Sequence: Synthetic polypeptide
QIQLQQSGPEVVKPGASVKISCKASGYTFTDYYINWVKQRPGQGLEWIGWIYPGGGHTKYNEKFKGEATLTVDTSSRTAYMQLSSLTSEDSAVYFCARYSNYYFDFWGHGTTLTVSS

SEQ ID NO: 122 - protein, 117 aa, synthetic construct
  REGION 1..117: note = Description of Artificial Sequence: Synthetic polypeptide
QIQLQQSGPELVKPGASVKISCKASGYTFTSYYIQWVKQRPGQGLEWIGWIYPGDGTTNYNENFKGKTTLTADKSSSTVYMLLSSLTSEDSAVYFCARYGLVPFDYWGQGTTLTVSS

SEQ ID NO: 123 - protein, 117 aa, synthetic construct
  REGION 1..117: note = Description of Artificial Sequence: Synthetic polypeptide
QIQLQQSGPELVKTGASVKISCKASGNTFNSNYIQWVKQRPGQGLEWIGWIYPGDGSTNYSEKFKGKTTLTADKSSSTAYMLVSSLTSEDSAVYFCARYGPVPFDYWGQGTTLSVSS

SEQ ID NO: 124 - DNA, 324 bp, synthetic construct
  misc_feature 1..324: note = Description of Artificial Sequence: Synthetic polynucleotide
  source 1..324: mol_type = other DNA
gatatccagatgacgcagagcccaagcagcctgagcgcgtccgtgggcgaccgcgtgacgatcacctgtagcgcgtcccagagcatcagcagcgactatctgcattggtatcagcagaaaccaggtaaagcccctaaactgctgatctaccggacctccaatctggcaagcggcgtgcctagccgtttcagcggtagcggctccggtaccgacttcacctttactatctccagcctgcagcctgaagacatcgcgacgtattattgtcagcagggtactagcctgcctcgcgccttcggccaggggaccaaactggaaatcaaa

SEQ ID NO: 125 - protein, 108 aa, synthetic construct
  REGION 1..108: note = Description of Artificial Sequence: Synthetic polypeptide
DIQMTQSPSSLSASVGDRVTITCSASQSISSDYLHWYQQKPGKAPKLLIYRTSNLASGVPSRFSGSGSGTDFTFTISSLQPEDIATYYCQQGTSLPRAFGQGTKLEIK

SEQ ID NO: 126 - DNA, 324 bp, synthetic construct
  misc_feature 1..324: note = Description of Artificial Sequence: Synthetic polynucleotide
  source 1..324: mol_type = other DNA
gatatccagatgacgcagagcccaagcagcctgagcgcgtccgtgggcgaccgcgtgacgatcacctgtcaggcgtcccagagcatcagcagcgactatctgcattggtatcagcagaaaccaggtaaagcccctaaactgctgatctaccggacctccaatctggaaaccggcgtgcctagccgtttcagcggtagcggctccggtaccgacttcacctttactatctccagcctgcagcctgaagacatcgcgacgtattattgtcagcagggtactagcctgcctcgcgccttcggccaggggaccaaactggaaatcaaa

SEQ ID NO: 127 - protein, 108 aa, synthetic construct
  REGION 1..108: note = Description of Artificial Sequence: Synthetic polypeptide
DIQMTQSPSSLSASVGDRVTITCQASQSISSDYLHWYQQKPGKAPKLLIYRTSNLETGVPSRFSGSGSGTDFTFTISSLQPEDIATYYCQQGTSLPRAFGQGTKLEIK

SEQ ID NO: 128 - DNA, 324 bp, synthetic construct
  misc_feature 1..324: note = Description of Artificial Sequence: Synthetic polynucleotide
  source 1..324: mol_type = other DNA
gatatccagatgacgcagagcccaagcagcctgagcgcgtccgtgggcgaccgcgtgacgatcacctgtcaggcgtcccagagcatcagcagcgactatctgcattggtatcagcagaaaccaggtaaagcccctaaactgctgatctaccggacctccaatctggaaagcggcgtgcctagccgtttcagcggtagcggctccggtaccgacttcacctttactatctccagcctgcagcctgaagacatcgcgacgtattattgtcagcagggtactagcctgcctcgcgccttcggccaggggaccaaactggaaatcaaa

SEQ ID NO: 129 - protein, 108 aa, synthetic construct
  REGION 1..108: note = Description of Artificial Sequence: Synthetic polypeptide
DIQMTQSPSSLSASVGDRVTITCQASQSISSDYLHWYQQKPGKAPKLLIYRTSNLESGVPSRFSGSGSGTDFTFTISSLQPEDIATYYCQQGTSLPRAFGQGTKLEIK

SEQ ID NO: 130 - DNA, 354 bp, synthetic construct
  misc_feature 1..354: note = Description of Artificial Sequence: Synthetic polynucleotide
  source 1..354: mol_type = other DNA
gaagtgcagctggtggaaagcggtggtggcctggtgcagccaggcggctccctgcgcctgagctgcgccgcaagcggtttcacctttagcgactactatatgtcctgggtgcgtcaggcgccaggtaaaggtctggaatgggtgtcatacatcagctccgggggcggtagcaagtactatccggacctggtgaaagggcgctttactatctcccgggataatgcaaaaaatagcctgtacctgcagatgagcagcctgcgggcggaagataccgccgtgtattactgtgcgcgtctgccgcattatttcgccatggattactggggccaggggaccctggtgaccgtgagcagc

SEQ ID NO: 131 - protein, 118 aa, synthetic construct
  REGION 1..118: note = Description of Artificial Sequence: Synthetic polypeptide
EVQLVESGGGLVQPGGSLRLSCAASGFTFSDYYMSWVRQAPGKGLEWVSYISSGGGSKYYPDLVKGRFTISRDNAKNSLYLQMSSLRAEDTAVYYCARLPHYFAMDYWGQGTLVTVSS

SEQ ID NO: 132 - DNA, 354 bp, synthetic construct
  misc_feature 1..354: note = Description of Artificial Sequence: Synthetic polynucleotide
  source 1..354: mol_type = other DNA
gaagtgcagctggtggaaagcggtggtggcctggtgcagccaggcggctccctgcgcctgagctgcgccgcaagcggtttcacctttagcgactactatatgtcctgggtgcgtcaggcgccaggtaaaggtctggaatgggtggcatacatcagctccgggggcggtagcagctactatccggacctggtgaaagggcgctttactatctcccgggataatgcaaaaaatagcctgtacctgcagatgcagagcctgcgggcggaagataccgccgtgtattactgtgcgcgtctgccgcattatttcgccatggattactggggccaggggaccctggtgaccgtgagcagc

SEQ ID NO: 133 - protein, 118 aa, synthetic construct
  REGION 1..118: note = Description of Artificial Sequence: Synthetic polypeptide
EVQLVESGGGLVQPGGSLRLSCAASGFTFSDYYMSWVRQAPGKGLEWVAYISSGGGSSYYPDLVKGRFTISRDNAKNSLYLQMQSLRAEDTAVYYCARLPHYFAMDYWGQGTLVTVSS

SEQ ID NO: 134 - DNA, 354 bp, synthetic construct
  misc_feature 1..354: note = Description of Artificial Sequence: Synthetic polynucleotide
  source 1..354: mol_type = other DNA
gaagtgcagctggtggaaagcggtggtggcctggtgcagccaggcggctccctgcgcctgagctgcgccgcaagcggtttcacctttagcgactactatatgtcctgggtgcgtcaggcgccaggtaaaggtctggaatgggtggcatacatcagctccgggggcggtagcagctactatccggacgctgtgaaagggcgctttactatctcccgggataatgcaaaacagagcctgtacctgcagatgcagagcctgcgggcggaagataccgccgtgtattactgtgcgcgtctgccgcattatttcgccatggattactggggccaggggaccctggtgaccgtgagcagc

SEQ ID NO: 135 - protein, 118 aa, synthetic construct
  REGION 1..118: note = Description of Artificial Sequence: Synthetic polypeptide
EVQLVESGGGLVQPGGSLRLSCAASGFTFSDYYMSWVRQAPGKGLEWVAYISSGGGSSYYPDAVKGRFTISRDNAKQSLYLQMQSLRAEDTAVYYCARLPHYFAMDYWGQGTLVTVSS

SEQ ID NO: 136 - DNA, 354 bp, synthetic construct
  misc_feature 1..354: note = Description of Artificial Sequence: Synthetic polynucleotide
  source 1..354: mol_type = other DNA
gaagtgcagctggtggaaagcggtggtggcctggtgcagccaggcggctccctgcgcctgagctgcgccgcaagcggtttcacctttagcgactactatatgtcctgggtgcgtcaggcgccaggtaaaggtctggaatgggtggcctacatcagctccgggggcggtagcagctactatccggacctggtgaaagggcgctttactatctcccgggataatgcaaaacagagcctgtacctgcagatgaacagcctgcgggcggaagataccgccgtgtattactgtgcgcgtctgccgcattatttcgccatggattactggggccaggggaccctggtgaccgtgagcagc

SEQ ID NO: 137 - protein, 118 aa, synthetic construct
  REGION 1..118: note = Description of Artificial Sequence: Synthetic polypeptide
EVQLVESGGGLVQPGGSLRLSCAASGFTFSDYYMSWVRQAPGKGLEWVAYISSGGGSSYYPDLVKGRFTISRDNAKQSLYLQMNSLRAEDTAVYYCARLPHYFAMDYWGQGTLVTVSS

SEQ ID NO: 138 - DNA, 354 bp, synthetic construct
  misc_feature 1..354: note = Description of Artificial Sequence: Synthetic polynucleotide
  source 1..354: mol_type = other DNA
gaagtgcagctggtggaaagcggtggtggcctggtgcagccaggcggctccctgcgcctgagctgcgccgcaagcggtttcacctttagcgactactatatgtcctgggtgcgtcaggcgccaggtaaaggtctggaatgggtggcctacatcagctccgggggcggtagcagctactatccggacctggtgaaagggcgctttactatctcccgggataatgcaaaacagagcctgtacctgcagatgcagagcctgcgggcggaagataccgccgtgtattactgtgcgcgtctgccgcattatttcgccatggattactggggccaggggaccctggtgaccgtgagcagc

SEQ ID NO: 139 - protein, 118 aa, synthetic construct
  REGION 1..118: note = Description of Artificial Sequence: Synthetic polypeptide
EVQLVESGGGLVQPGGSLRLSCAASGFTFSDYYMSWVRQAPGKGLEWVAYISSGGGSSYYPDLVKGRFTISRDNAKQSLYLQMQSLRAEDTAVYYCARLPHYFAMDYWGQGTLVTVSS

SEQ ID NO: 140 - DNA, 645 bp, synthetic construct
  misc_feature 1..645: note = Description of Artificial Sequence: Synthetic polynucleotide
  source 1..645: mol_type = other DNA
gatatccagatgacgcagagcccaagcagcctgagcgcgtccgtgggcgaccgcgtgacgatcacctgtagcgcgtcccagagcatcagcagcgactatctgcattggtatcagcagaaaccaggtaaagcccctaaactgctgatctaccggacctccaatctggcaagcggcgtgcctagccgtttcagcggtagcggctccggtaccgacttcacctttactatctccagcctgcagcctgaagacatcgcgacgtattattgtcagcagggtactagcctgcctcgcgccttcggccaggggaccaaactggaaatcaaacgtactgtggctgcaccatctgtcttcatcttcccgccatctgatgagcaattgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 141 - protein, 215 aa, synthetic construct
  REGION 1..215: note = Description of Artificial Sequence: Synthetic polypeptide
DIQMTQSPSSLSASVGDRVTITCSASQSISSDYLHWYQQKPGKAPKLLIYRTSNLASGVPSRFSGSGSGTDFTFTISSLQPEDIATYYCQQGTSLPRAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - DNA, 1332 bp, synthetic construct
  misc_feature 1..1332: note = Description of Artificial Sequence: Synthetic polynucleotide
  source 1..1332: mol_type = other DNA
gaagtgcagctggtggaaagcggtggtggcctggtgcagccaggcggctccctgcgcctgagctgcgccgcaagcggtttcacctttagcgactactatatgtcctgggtgcgtcaggcgccaggtaaaggtctggaatgggtgtcatacatcagctccgggggcggtagcaagtactatccggacctggtgaaagggcgctttactatctcccgggataatgcaaaaaatagcctgtacctgcagatgagcagcctgcgggcggaagataccgccgtgtattactgtgcgcgtctgccgcattatttcgccatggattactggggccaggggaccctggtgaccgtgagcagcgcctccacaaagggcccttccgtgttccccctggccccttgctcccggtccacctccgagtctaccgccgctctgggctgcctggtcaaggactacttccccgagcccgtgaccgtgtcctggaactctggcgccctgacctccggcgtgcacaccttccctgctgtgctgcagtcctccggcctgtactccctgtcctccgtcgtgaccgtgccctcctctagcctgggcaccaagacctacacctgtaacgtggaccacaagccctccaacaccaaggtggacaagcgggtggaatctaagtacggccctccctgccccccctgccctgcccctgaatttctgggcggaccctccgtgttcctgttccccccaaagcccaaggacaccctgatgatctcccggacccccgaagtgacctgcgtggtggtggacgtgtcccaggaagatcccgaggtccagtttaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgcctccaagccaggaagagatgaccaagaaccaggtgtccctgacctgtctggtcaagggcttctacccctccgatatcgccgtggaatgggagtccaacggccagcccgagaacaactacaagaccaccccccctgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgagcctgggc

SEQ ID NO: 143 - protein, 444 aa, synthetic construct
  REGION 1..444: note = Description of Artificial Sequence: Synthetic polypeptide
EVQLVESGGGLVQPGGSLRLSCAASGFTFSDYYMSWVRQAPGKGLEWVSYISSGGGSKYYPDLVKGRFTISRDNAKNSLYLQMSSLRAEDTAVYYCARLPHYFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 144 - DNA, 645 bp, synthetic construct
  misc_feature 1..645: note = Description of Artificial Sequence: Synthetic polynucleotide
  source 1..645: mol_type = other DNA
gacatccagatgacccagagcccaagcagcctgagcgccagcgtgggcgaccgcgtgaccatcacctgccaggccagccagagcatcagcagcgactacctgcactggtaccagcagaagccaggcaaggccccaaagctgctgatctaccgcaccagcaacctggagaccggcgtgccaagccgcttcagcggcagcggcagcggcaccgacttcaccttcaccatcagcagcctgcagccagaggacatcgccacctactactgccagcagggcaccagcctgccacgcgccttcggccagggcaccaagctggagatcaagcgtactgtggctgcaccatctgtcttcatcttcccgccatctgatgagcaattgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 145 - protein, 215 aa, synthetic construct
  REGION 1..215: note = Description of Artificial Sequence: Synthetic polypeptide
DIQMTQSPSSLSASVGDRVTITCQASQSISSDYLHWYQQKPGKAPKLLIYRTSNLETGVPSRFSGSGSGTDFTFTISSLQPEDIATYYCQQGTSLPRAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - DNA, 1332 bp, synthetic construct
  misc_feature 1..1332: note = Description of Artificial Sequence: Synthetic polynucleotide
  source 1..1332: mol_type = other DNA
gaagtgcagctggtggaaagcggcggaggcctggtgcagccaggcggcagcctgagactgagctgcgccgccagcggcttcaccttcagcgactactacatgagctgggtgcgccaggccccaggcaagggcctggagtgggtggcctacatcagcagcggcggcggcagcagctactacccagacctggtgaagggccgcttcaccatcagccgcgacaacgccaagaacagcctgtacctgcagatgcagagcctgcgcgccgaggacaccgccgtgtactactgcgcccgcctgccacactacttcgccatggactactggggccagggcaccctggtgaccgtgagcagcgcctccacaaagggcccttccgtgttccccctggccccttgctcccggtccacctccgagtctaccgccgctctgggctgcctggtcaaggactacttccccgagcccgtgaccgtgtcctggaactctggcgccctgacctccggcgtgcacaccttccctgctgtgctgcagtcctccggcctgtactccctgtcctccgtcgtgaccgtgccctcctctagcctgggcaccaagacctacacctgtaacgtggaccacaagccctccaacaccaaggtggacaagcgggtggaatctaagtacggccctccctgccccccctgccctgcccctgaatttctgggcggaccctccgtgttcctgttccccccaaagcccaaggacaccctgatgatctcccggacccccgaagtgacctgcgtggtggtggacgtgtcccaggaagatcccgaggtccagtttaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgcctccaagccaggaagagatgaccaagaaccaggtgtccctgacctgtctggtcaagggcttctacccctccgatatcgccgtggaatgggagtccaacggccagcccgagaacaactacaagaccaccccccctgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgagcctgggc

SEQ ID NO: 147 - protein, 444 aa, synthetic construct
  REGION 1..444: note = Description of Artificial Sequence: Synthetic polypeptide
EVQLVESGGGLVQPGGSLRLSCAASGFTFSDYYMSWVRQAPGKGLEWVAYISSGGGSSYYPDLVKGRFTISRDNAKNSLYLQMQSLRAEDTAVYYCARLPHYFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 148 - DNA, 1332 bp, synthetic construct
  misc_feature 1..1332: note = Description of Artificial Sequence: Synthetic polynucleotide
  source 1..1332: mol_type = other DNA
gaggtgcagctggtggagagcggcggcggcctggtgcagccaggtggtagcctgcgcctgagctgcgccgccagcggcttcaccttcagcgactactacatgagctgggtgcgccaggctccaggcaagggtctggaatgggtggcctacatcagcagcggcggcggcagcagctactacccagacgccgtgaagggccgcttcaccatcagccgcgacaacgccaagcagagcctgtacctgcagatgcagagcctgcgcgccgaggacaccgccgtgtactactgcgcccgcctgccacactacttcgccatggactactggggccagggcaccctggtgaccgtgagcagcgcctccacaaagggcccttccgtgttccccctggccccttgctcccggtccacctccgagtctaccgccgctctgggctgcctggtcaaggactacttccccgagcccgtgaccgtgtcctggaactctggcgccctgacctccggcgtgcacaccttccctgctgtgctgcagtcctccggcctgtactccctgtcctccgtcgtgaccgtgccctcctctagcctgggcaccaagacctacacctgtaacgtggaccacaagccctccaacaccaaggtggacaagcgggtggaatctaagtacggccctccctgccccccctgccctgcccctgaatttctgggcggaccctccgtgttcctgttccccccaaagcccaaggacaccctgatgatctcccggacccccgaagtgacctgcgtggtggtggacgtgtcccaggaagatcccgaggtccagtttaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgcctccaagccaggaagagatgaccaagaaccaggtgtccctgacctgtctggtcaagggcttctacccctccgatatcgccgtggaatgggagtccaacggccagcccgagaacaactacaagaccaccccccctgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgagcctgggc

SEQ ID NO: 149 - protein, 444 aa, synthetic construct
  REGION 1..444: note = Description of Artificial Sequence: Synthetic polypeptide
EVQLVESGGGLVQPGGSLRLSCAASGFTFSDYYMSWVRQAPGKGLEWVAYISSGGGSSYYPDAVKGRFTISRDNAKQSLYLQMQSLRAEDTAVYYCARLPHYFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 150 - DNA, 645 bp, synthetic construct
  misc_feature 1..645: note = Description of Artificial Sequence: Synthetic polynucleotide
  source 1..645: mol_type = other DNA
gacatccagatgacccagagcccaagcagcctgagcgccagcgtgggcgaccgcgtgaccatcacctgccaggccagccagagcatcagcagcgactacctgcactggtaccagcagaagccaggcaaggccccaaagctgctgatctaccgcaccagcaacctggagagcggcgtgccaagccgcttcagcggcagcggcagcggcaccgacttcaccttcaccatcagcagcctgcagccagaggacatcgccacctactactgccagcagggcaccagcctgccacgcgccttcggccagggcaccaagctggagatcaagcgtactgtggctgcaccatctgtcttcatcttcccgccatctgatgagcaattgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 151 - protein, 215 aa, synthetic construct
  REGION 1..215: note = Description of Artificial Sequence: Synthetic polypeptide
DIQMTQSPSSLSASVGDRVTITCQASQSISSDYLHWYQQKPGKAPKLLIYRTSNLESGVPSRFSGSGSGTDFTFTISSLQPEDIATYYCQQGTSLPRAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - DNA, 1332 bp, synthetic construct
  misc_feature 1..1332: note = Description of Artificial Sequence: Synthetic polynucleotide
  source 1..1332: mol_type = other DNA
gaagtgcagctggtggaaagcggtggtggcctggtgcagccaggcggctccctgcgcctgagctgcgccgcaagcggtttcacctttagcgactactatatgtcctgggtgcgtcaggcgccaggtaaaggtctggaatgggtggcctacatcagctccgggggcggtagcagctactatccggacctggtgaaagggcgctttactatctcccgggataatgcaaaacagagcctgtacctgcagatgaacagcctgcgggcggaagataccgccgtgtattactgtgcgcgtctgccgcattatttcgccatggattactggggccaggggaccctggtgaccgtgagcagcgcctccacaaagggcccttccgtgttccccctggccccttgctcccggtccacctccgagtctaccgccgctctgggctgcctggtcaaggactacttccccgagcccgtgaccgtgtcctggaactctggcgccctgacctccggcgtgcacaccttccctgctgtgctgcagtcctccggcctgtactccctgtcctccgtcgtgaccgtgccctcctctagcctgggcaccaagacctacacctgtaacgtggaccacaagccctccaacaccaaggtggacaagcgggtggaatctaagtacggccctccctgccccccctgccctgcccctgaatttctgggcggaccctccgtgttcctgttccccccaaagcccaaggacaccctgatgatctcccggacccccgaagtgacctgcgtggtggtggacgtgtcccaggaagatcccgaggtccagtttaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgcctccaagccaggaagagatgaccaagaaccaggtgtccctgacctgtctggtcaagggcttctacccctccgatatcgccgtggaatgggagtccaacggccagcccgagaacaactacaagaccaccccccctgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgagcctgggc

SEQ ID NO: 153 - protein, 444 aa, synthetic construct
  REGION 1..444: note = Description of Artificial Sequence: Synthetic polypeptide
EVQLVESGGGLVQPGGSLRLSCAASGFTFSDYYMSWVRQAPGKGLEWVAYISSGGGSSYYPDLVKGRFTISRDNAKQSLYLQMNSLRAEDTAVYYCARLPHYFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 154 - DNA, 1332 bp, synthetic construct
  misc_feature 1..1332: note = Description of Artificial Sequence: Synthetic polynucleotide
  source 1..1332: mol_type = other DNA
gaggtgcagctggtggagagcggcggcggcctggtgcagccaggtggtagcctgcgcctgagctgcgccgccagcggcttcaccttcagcgactactacatgagctgggtgcgccaggctccaggcaagggtctggaatgggtggcctacatcagcagcggcggcggcagcagctactacccagacctggtgaagggccgcttcaccatcagccgcgacaacgccaagcagagcctgtacctgcagatgcagagcctgcgcgccgaggacaccgccgtgtactactgcgcccgcctgccacactacttcgccatggactactggggccagggcaccctggtgaccgtgagcagcgcctccacaaagggcccttccgtgttccccctggccccttgctcccggtccacctccgagtctaccgccgctctgggctgcctggtcaaggactacttccccgagcccgtgaccgtgtcctggaactctggcgccctgacctccggcgtgcacaccttccctgctgtgctgcagtcctccggcctgtactccctgtcctccgtcgtgaccgtgccctcctctagcctgggcaccaagacctacacctgtaacgtggaccacaagccctccaacaccaaggtggacaagcgggtggaatctaagtacggccctccctgccccccctgccctgcccctgaatttctgggcggaccctccgtgttcctgttccccccaaagcccaaggacaccctgatgatctcccggacccccgaagtgacctgcgtggtggtggacgtgtcccaggaagatcccgaggtccagtttaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgcctccaagccaggaagagatgaccaagaaccaggtgtccctgacctgtctggtcaagggcttctacccctccgatatcgccgtggaatgggagtccaacggccagcccgagaacaactacaagaccaccccccctgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgagcctgggc

SEQ ID NO: 155 - protein, 444 aa, synthetic construct
  REGION 1..444: note = Description of Artificial Sequence: Synthetic polypeptide
EVQLVESGGGLVQPGGSLRLSCAASGFTFSDYYMSWVRQAPGKGLEWVAYISSGGGSSYYPDLVKGRFTISRDNAKQSLYLQMQSLRAEDTAVYYCARLPHYFAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 156 - DNA, 645 bp, synthetic construct
  misc_feature 1..645: note = Description of Artificial Sequence: Synthetic polynucleotide
  source 1..645: mol_type = other DNA
gagatcgtgctgacacagagccctaccacaatggccgcctctccaggcgagaagatcaccatcacatgtagcgccagcagcagcatcagcagcgactacctgcactggtatcagcagaagcctggcttcagccccgagctgctgatctacagaacaagcaatctggccagcggcgtgccagccagattttctggttctggcagcggcaccagctacagcctgacaatcggatccatggaagccgaggacgtggccacctattactgtcagcagggcacaagcctgcctagagcctttggcggaggcaccaagctggaaatcaagcgtactgtggctgcaccatctgtcttcatcttcccgccatctgatgagcaattgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 157 - protein, 215 aa, synthetic construct
  REGION 1..215: note = Description of Artificial Sequence: Synthetic polypeptide
EIVLTQSPTTMAASPGEKITITCSASSSISSDYLHWYQQKPGFSPELLIYRTSNLASGVPARFSGSGSGTSYSLTIGSMEAEDVATYYCQQGTSLPRAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - DNA, 1341 bp, synthetic construct
  misc_feature 1..1341: note = Description of Artificial Sequence: Synthetic polynucleotide
  source 1..1341: mol_type = other DNA
gaagtgcagctggtggaatctggcggaggacttgttcaacctggcggcagcctgaaactgtcttgtgccgccagcggcttcaccttcagcgactactacatgagctgggtccgacagacccctgagaagagactggaatgggtcgcctacatcagctctggcggcggaagcagctactaccctgatagcgtgaagggcagattcaccatcagccgggacaacaccaagaacaccctgtacctgcagatgtccagcctgaagtctgaggacaccgccgtgtactactgtgccagactgcctcactacttcgccatggattattggggccagggcaccagcgtgaccgtttcttctgcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagcgcgttgagcccaaatcttgtgacaaaactcacacatgcccaccgtgcccagcacctgaactactagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtaagtttgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 159 - protein, 447 aa, synthetic construct
  REGION 1..447: note = Description of Artificial Sequence: Synthetic polypeptide
EVQLVESGGGLVQPGGSLKLSCAASGFTFSDYYMSWVRQTPEKRLEWVAYISSGGGSSYYPDSVKGRFTISRDNTKNTLYLQMSSLKSEDTAVYYCARLPHYFAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - DNA, 1341 bp, synthetic construct
  misc_feature 1..1341: note = Description of Artificial Sequence: Synthetic polynucleotide
  source 1..1341: mol_type = other DNA
gaagtgcagctggtggaatctggcggaggacttgttcaacctggcggcagcctgaaactgtcttgtgccgccagcggcttcaccttcagcgactactacatgagctgggtccgacagacccctgagaagagactggaatgggtcgcctacatcagctctggcggcggaagcagctactaccctgatagcgtgaagggcagattcaccatcagccgggacaacaccaagaacaccctgtacctgcagatgtccagcctgaagtctgaggacaccgccgtgtactactgtgccagactgcctcactacttcgccatggattgttggggccagggcacatctgtgaccgttagttctgcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagcgcgttgagcccaaatcttgtgacaaaactcacacatgcccaccgtgcccagcacctgaagccgctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtaagtttgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 161 - protein, 447 aa, synthetic construct
  REGION 1..447: note = Description of Artificial Sequence: Synthetic polypeptide
EVQLVESGGGLVQPGGSLKLSCAASGFTFSDYYMSWVRQTPEKRLEWVAYISSGGGSSYYPDSVKGRFTISRDNTKNTLYLQMSSLKSEDTAVYYCARLPHYFAMDCWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - DNA, 1332 bp, synthetic construct
  misc_feature 1..1332: note = Description of Artificial Sequence: Synthetic polynucleotide
  source 1..1332: mol_type = other DNA
gaagtgcagctggtggaatctggcggaggacttgttcaacctggcggcagcctgaaactgtcttgtgccgccagcggcttcaccttcagcgactactacatgagctgggtccgacagacccctgagaagagactggaatgggtcgcctacatcagctctggcggcggaagcagctactaccctgatagcgtgaagggcagattcaccatcagccgggacaacaccaagaacaccctgtacctgcagatgtccagcctgaagtctgaggacaccgccgtgtactactgtgccagactgcctcactacttcgccatggattattggggccagggcaccagcgtgaccgtttcttctgcctccacaaagggcccttccgtgttccccctggccccttgctcccggtccacctccgagtctaccgccgctctgggctgcctggtcaaggactacttccccgagcccgtgaccgtgtcctggaactctggcgccctgacctccggcgtgcacaccttccctgctgtgctgcagtcctccggcctgtactccctgtcctccgtcgtgaccgtgccctcctctagcctgggcaccaagacctacacctgtaacgtggaccacaagccctccaacaccaaggtggacaagcgggtggaatctaagtacggccctccctgccccccctgccctgcccctgaatttctgggcggaccctccgtgttcctgttccccccaaagcccaaggacaccctgatgatctcccggacccccgaagtgacctgcgtggtggtggacgtgtcccaggaagatcccgaggtccagtttaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgcctccaagccaggaagagatgaccaagaaccaggtgtccctgacctgtctggtcaagggcttctacccctccgatatcgccgtggaatgggagtccaacggccagcccgagaacaactacaagaccaccccccctgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgagcctgggc

SEQ ID NO: 163 - protein, 444 aa, synthetic construct
  REGION 1..444: note = Description of Artificial Sequence: Synthetic polypeptide
EVQLVESGGGLVQPGGSLKLSCAASGFTFSDYYMSWVRQTPEKRLEWVAYISSGGGSSYYPDSVKGRFTISRDNTKNTLYLQMSSLKSEDTAVYYCARLPHYFAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 164 - protein, 12 aa, synthetic construct
  REGION 1..12: note = Description of Artificial Sequence: Synthetic peptide
SASQSISSDYLH

SEQ ID NO: 165 - protein, 12 aa, synthetic construct
  REGION 1..12: note = Description of Artificial Sequence: Synthetic peptide
QASQSISSDYLH

SEQ ID NO: 166 - protein, 7 aa, synthetic construct
  REGION 1..7: note = Description of Artificial Sequence: Synthetic peptide
RTSNLET

SEQ ID NO: 167 - protein, 7 aa, synthetic construct
  REGION 1..7: note = Description of Artificial Sequence: Synthetic peptide
RTSNLES

(19) United States
(12) Patent Application Publication
(10) Pub. No.: US 2026/0070984 A1
(43) Pub. Date: Mar. 12, 2026

(54) ANTI-PD-1 ANTIBODIES

(71) Applicant: Boehringer Ingelheim International GmbH, Ingelheim am Rhein (DE)

(72) Inventors: David A. BLAIR, Sandy Hook, CT (US); Nicole K. GARAFFA, Trumbull, CT (US); Pankaj GUPTA, Scarsdale, NY (US); Priyanka GUPTA, Newtown, CT (US); Fei HAN, Acton, MA (US); Aaron Timothy KARLAK, Beacon Falls, CT (US); Dongmei LIU, Oxford, CT (US); Mouhamadou Lamine MBOW, Echichens (CH); Joseph A. MOZDZIERZ, Mahopac, NY (US); Kerry L.M. RALPH, Bethel, CT (US); Abdulsalam SHAABAN, Ridgefield, CT (US); Helen Haixia WU, Danbury, CT (US); Guangwei YANG, Cheshire, CT (US)

(21) Appl. No.: 19/188,480

(22) Filed: Apr. 24, 2025

Related U.S. Application Data
(63) Continuation of application No. 17/328,050, filed on May 24, 2021, now Pat. No. 12,312,405.
(60) Provisional application No. 63/029,962, filed on May 26, 2020.
(60) Provisional application No. 63/039,686, filed on Jun. 16, 2020.

(51) Int. Cl. C07K 16/28; A61K 39/00

(52) U.S. Cl. CPC C07K 16/2818; C07K 16/2803; A61K 2039/505; C07K 2317/24; C07K 2317/31; C07K 2317/75; C07K 2317/92

(57) ABSTRACT

The present invention relates to new anti-PD-1 (Programmed cell death 1) antibodies and antigen-binding fragments thereof for therapeutic and diagnostic methods and compositions using them.

### RELATED APPLICATION DISCLOSURE

This application is a continuation of U.S. application Ser. No. 17/328,050, filed May 24, 2021, which is hereby incorporated by reference herein in its entirety and which claims the benefit of U.S. Provisional Application No. 63/029,962, filed May 26, 2020, and U.S. Provisional Application No. 63/039,686, filed Jun. 16, 2020.

### SEQUENCE LISTING

The instant application contains a Sequence Listing which has been submitted electronically in XML format and is hereby incorporated by reference in its entirety. Said XML copy, created on Apr. 17, 2025, is named 09-0701-US-4_2025-04-25-SL.xml and is 239,570 bytes in size.

### FIELD OF THE INVENTION

This invention generally relates to anti-PD-1 (Programmed cell death 1) antibodies for therapeutic and diagnostic use. More specifically, anti-PD-1 antibodies and methods of use for the treatment of various diseases or disorders characterized by cells expressing PD-1 are disclosed. Pharmaceutical compositions and kits comprising the anti-PD-1 antibodies are also disclosed.

### BACKGROUND OF THE INVENTION

Programmed cell death 1, also known as PD-1 and CD279 (cluster of differentiation 279), is a cell surface receptor protein expressed primarily on T-cells, but also on other immune cells. The PD-1 pathway is a key regulator in the induction and maintenance of immunological tolerance. The protein functions as an “immune checkpoint” inhibitor, i.e. it acts to modulate the activity of cells in the immune system so as to regulate and limit autoimmune diseases. PD-1 has two ligands, PD-L1 and PD-L2, which interact with the cell surface receptor. On binding, PD-1 induces an intracellular signal, which negatively regulates T-cell responses. On the surface of activated T cells, PD-1 expression is upregulated after the recognition of peripheral antigens by T cells; subsequently, the elevated binding of PD-1 to PD-L1 and PD-L2 becomes a key step for downstream inhibitory signaling. PD-1 is also associated with increased Treg-cell proliferation and enhanced immunosuppressive function.

It has been recently understood that many cancers can protect themselves from the immune system by modifying “immune checkpoint” inhibitors and thus avoid detection. PD-1 inhibitors, a new class of drugs that block PD-1, activate the immune system to attack tumors and are used to treat certain types of cancer.

In contrast, defective PD-1 inhibitory functions have also been associated with pathophysiology of immune-mediated diseases, and expression of PD-1 or it is ligands may be dysregulated or not fully engaged in certain autoimmune indications. Induction of PD-1 activation and the use of the PD-1/PD-L1 and/or PD-L2 system thus represent an alternative approach to suppress the immune response and provide treatments for various immune and inflammatory disorders.

There is therefore a need for therapies, which induce the PD-1 pathway, enhance suppressive function and provide treatments for immune and inflammatory disorders controlled by the PD-1/PD-L1 and/or PD-L2 system. In particular, there is a need for biological therapeutics, such as antibodies, which modulate the interaction between PD-1 and PD-L1 or PD-L2 without blocking such interaction.

### BRIEF SUMMARY OF THE INVENTION

The present invention provides antibodies that specifically bind to human PD-1. In one aspect of the invention, the antibodies of the present invention do not block the interaction between PD-1 and PD-L1. In one aspect of the invention, the antibodies of the present invention enhance the interaction between PD-1 and PD-L1. In one aspect of the invention, the antibodies of the present invention activate the PD-1 signaling pathway. In one aspect of the invention, the antibodies of the present invention are anti-PD-1 agonist antibodies. The antibodies of the invention are useful, for example, for the treatment and/or prevention of diseases or disorders that can be alleviated by modulating the interaction between PD-1 and PD-L1, in particular by activating the PD-1 pathway.

In one aspect, the present invention provides an anti-PD-1 antibody, in particular a monoclonal anti-PD-1 antibody, for example a humanized monoclonal anti-PD-1 antibody, having one or more of the properties described herein below. In one aspect, an anti-PD-1 antibody of the present invention binds to purified recombinant human PD-1 at high affinity, for example 20 nM or less, for example 10 nM or less, for example 5 nM of less. In one aspect, an anti-PD-1 antibody of the present invention binds to purified recombinant cynomologus PD-1 at an affinity of 50 nM or less. In one aspect, an anti-PD-1 antibody of the present invention selectively binds to PD-1, in particular human PD-1. In one aspect, an antibody of the present invention does not bind to mouse, rat, or rabbit PD-1. In one aspect, an anti-PD-1 antibody of the present invention does not block the binding of PD-L1 to PD-1. In one aspect, an anti-PD-1 antibody of the present invention enhances the binding of PD-L1 to PD-1. In one aspect, an anti-PD-1 antibody of the present invention attenuates T cell activity in a functional cell assay, for example by inhibition of IFNγ production, inhibition of IL-17A production or inhibition of IL-21 production. In one aspect, an anti-PD-1 antibody of the present invention inhibits human cell accumulation in a mouse model and reduces the levels of human inflammatory cytokines in the mouse model. In one aspect, an anti-PD-1 antibody of the present invention has favorable pharmacokinetic properties. In one aspect, an anti-PD-1 antibody of the present invention has favorable biophysical properties, for example yield, quality, stability or solubility. In one aspect, the present invention provides antigen-binding fragments of an antibody of the present invention.

In one embodiment, the present invention provides an anti-PD-1 antibody or antigen-binding fragment thereof comprising:
a heavy chain variable region comprising the amino acid sequence of SEQ ID NO: 43 (H-CDR1); the amino acid sequence of SEQ ID NO: 44 (H-CDR2); and the amino acid sequence of SEQ ID NO: 45 (H-CDR3), and
a light chain variable region comprising the amino acid sequence of SEQ ID NO: 1 (L-CDR1); the amino acid sequence of SEQ ID NO: 2 (L-CDR2); and the amino acid sequence of SEQ ID NO: 29 (L-CDR3),
or
a heavy chain variable region comprising the amino acid sequence of SEQ ID NO: 43 (H-CDR1); the amino acid sequence of SEQ ID NO: 46 (H-CDR2); and the amino acid sequence of SEQ ID NO: 45 (H-CDR3), and
a light chain variable region comprising the amino acid sequence of SEQ ID NO: 1 (L-CDR1); the amino acid sequence of SEQ ID NO: 2 (L-CDR2); and the amino acid sequence of SEQ ID NO: 3 (L-CDR3),
or
a heavy chain variable region comprising the amino acid sequence of SEQ ID NO: 47 (H-CDR1); the amino acid sequence of SEQ ID NO: 48 (H-CDR2); and the amino acid sequence of SEQ ID NO: 49 (H-CDR3), and
a light chain variable region comprising the amino acid sequence of SEQ ID NO: 4 (L-CDR1); the amino acid sequence of SEQ ID NO: 5 (L-CDR2); and the amino acid sequence of SEQ ID NO: 6 (L-CDR3),
or
a heavy chain variable region comprising the amino acid sequence of SEQ ID NO: 50 (H-CDR1); the amino acid sequence of SEQ ID NO: 51 (H-CDR2); and the amino acid sequence of SEQ ID NO: 52 (H-CDR3), and
a light chain variable region comprising the amino acid sequence of SEQ ID NO: 7 (L-CDR1); the amino acid sequence of SEQ ID NO: 8 (L-CDR2); and the amino acid sequence of SEQ ID NO: 9 (L-CDR3),
or
a heavy chain variable region comprising the amino acid sequence of SEQ ID NO: 53 (H-CDR1); the amino acid sequence of SEQ ID NO: 54 (H-CDR2); and the amino acid sequence of SEQ ID NO: 55 (H-CDR3), and
a light chain variable region comprising the amino acid sequence of SEQ ID NO: 10 (L-CDR1); the amino acid sequence of SEQ ID NO: 11 (L-CDR2); and the amino acid sequence of SEQ ID NO: 12 (L-CDR3),
or
a heavy chain variable region comprising the amino acid sequence of SEQ ID NO: 56 (H-CDR1); the amino acid sequence of SEQ ID NO: 57 (H-CDR2); and the amino acid sequence of SEQ ID NO: 58 (H-CDR3), and
a light chain variable region comprising the amino acid sequence of SEQ ID NO: 13 (L-CDR1); the amino acid sequence of SEQ ID NO: 14 (L-CDR2); and the amino acid sequence of SEQ ID NO: 15 (L-CDR3),
or
a heavy chain variable region comprising the amino acid sequence of SEQ ID NO: 59 (H-CDR1); the amino acid sequence of SEQ ID NO: 60 (H-CDR2); and the amino acid sequence of SEQ ID NO: 61 (H-CDR3), and
a light chain variable region comprising the amino acid sequence of SEQ ID NO: 16 (L-CDR1); the amino acid sequence of SEQ ID NO: 17 (L-CDR2); and the amino acid sequence of SEQ ID NO: 18 (L-CDR3),
or
a heavy chain variable region comprising the amino acid sequence of SEQ ID NO: 62 (H-CDR1); the amino acid sequence of SEQ ID NO: 63 (H-CDR2); and the amino acid sequence of SEQ ID NO: 64 (H-CDR3), and
a light chain variable region comprising the amino acid sequence of SEQ ID NO: 19 (L-CDR1); the amino acid sequence of SEQ ID NO: 20 (L-CDR2); and the amino acid sequence of SEQ ID NO: 21 (L-CDR3),
or
a heavy chain variable region comprising the amino acid sequence of SEQ ID NO: 65 (H-CDR1); the amino acid sequence of SEQ ID NO: 66 (H-CDR2); and the amino acid sequence of SEQ ID NO: 67 (H-CDR3), and
a light chain variable region comprising the amino acid sequence of SEQ ID NO: 22 (L-CDR1); the amino acid sequence of SEQ ID NO: 23 (L-CDR2); and the amino acid sequence of SEQ ID NO: 24 (L-CDR3),
or
a heavy chain variable region comprising the amino acid sequence of SEQ ID NO: 68 (H-CDR1); the amino acid sequence of SEQ ID NO: 69 (H-CDR2); and the amino acid sequence of SEQ ID NO: 70 (H-CDR3), and
a light chain variable region comprising the amino acid sequence of SEQ ID NO: 25 (L-CDR1); the amino acid sequence of SEQ ID NO: 26 (L-CDR2); and the amino acid sequence of SEQ ID NO: 27 (L-CDR3),
or
a heavy chain variable region comprising the amino acid sequence of SEQ ID NO: 71 (H-CDR1); the amino acid sequence of SEQ ID NO: 72 (H-CDR2); and the amino acid sequence of SEQ ID NO: 58 (H-CDR3), and
a light chain variable region comprising the amino acid sequence of SEQ ID NO: 28 (L-CDR1); the amino acid sequence of SEQ ID NO: 14 (L-CDR2); and the amino acid sequence of SEQ ID NO: 29 (L-CDR3),
or
a heavy chain variable region comprising the amino acid sequence of SEQ ID NO: 73 (H-CDR1); the amino acid sequence of SEQ ID NO: 74 (H-CDR2); and the amino acid sequence of SEQ ID NO: 75 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 76 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 78 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 76 (H-CDR2); and the amino acid sequence of SEQ ID NO: 77 (H-CDR3), and
a light chain variable region comprising the amino acid sequence of SEQ ID NO: 164 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 166 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 78 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 166 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 167 (L-CDR2); and the amino acid sequence of SEQ ID NO: 32 (L-CDR3),
or
a heavy chain variable region comprising the amino acid sequence of SEQ ID NO: 80 (H-CDR1); the amino acid sequence of SEQ ID NO: 81 (H-CDR2); and the amino acid sequence of SEQ ID NO: 82 (H-CDR3), and
a light chain variable region comprising the amino acid sequence of SEQ ID NO: 33 (L-CDR1); the amino acid sequence of SEQ ID NO: 14 (L-CDR2); and the amino acid sequence of SEQ ID NO: 34 (L-CDR3),
or
a heavy chain variable region comprising the amino acid sequence of SEQ ID NO: 83 (H-CDR1); the amino acid sequence of SEQ ID NO: 84 (H-CDR2); and the amino acid sequence of SEQ ID NO: 85 (H-CDR3), and
a light chain variable region comprising the amino acid sequence of SEQ ID NO: 16 (L-CDR1); the amino acid sequence of SEQ ID NO: 35 (L-CDR2); and the amino acid sequence of SEQ ID NO: 36 (L-CDR3),
or
a heavy chain variable region comprising the amino acid sequence of SEQ ID NO: 86 (H-CDR1); the amino acid sequence of SEQ ID NO: 87 (H-CDR2); and the amino acid sequence of SEQ ID NO: 88 (H-CDR3), and
a light chain variable region comprising the amino acid sequence of SEQ ID NO: 37 (L-CDR1); the amino acid sequence of SEQ ID NO: 38 (L-CDR2); and the amino acid sequence of SEQ ID NO: 39 (L-CDR3),
or
a heavy chain variable region comprising the amino acid sequence of SEQ ID NO: 89 (H-CDR1); the amino acid sequence of SEQ ID NO: 90 (H-CDR2); and the amino acid sequence of SEQ ID NO: 91 (H-CDR3), and
a light chain variable region comprising the amino acid sequence of SEQ ID NO: 40 (L-CDR1); the amino acid sequence of SEQ ID NO: 41 (L-CDR2); and the amino acid sequence of SEQ ID NO: 42 (L-CDR3).

In one embodiment, the present invention provides an anti-PD-1 antibody or antigen-binding fragment comprising:
a heavy chain variable region comprising the amino acid sequence of SEQ ID NO: 73 (H-CDR1); the amino acid sequence of SEQ ID NO: 74 (H-CDR2); and the amino acid sequence of SEQ ID NO: 75 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 76 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 78 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 76 (H-CDR2); and the amino acid sequence of SEQ ID NO: 77 (H-CDR3), and
a light chain variable region comprising the amino acid sequence of SEQ ID NO: 164 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 166 (L-CDR2); and the amino acid sequence of SEQ ID NO: 29 (L-CDR3),
or
a heavy chain variable region comprising the amino acid sequence of SEQ ID NO: 73 (H-CDR1); the amino acid sequence of SEQ ID NO: 78 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 166 (L-CDR2); and the amino acid sequence of SEQ ID NO: 32 (L-CDR3),
or
a heavy chain variable region comprising the amino acid sequence of SEQ ID NO: 73 (H-CDR1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 167 (L-CDR2); and the amino acid sequence of SEQ ID NO: 32 (L-CDR3).

In one embodiment, the present invention provides an anti-PD-1 antibody or antigen-binding fragment comprising:
a heavy chain variable region comprising any one of:
the amino acid sequence SEQ ID NO: 73 (H-CDR1); the amino acid sequence of SEQ ID NO: 74 (H-CDR2); and the amino acid sequence of SEQ ID NO: 75 (H-CDR3),
the amino acid sequence SEQ ID NO: 73 (H-CDR1); the amino acid sequence of SEQ ID NO: 76 (H-CDR2); and the amino acid sequence of SEQ ID NO: 77 (H-CDR3),
the amino acid sequence SEQ ID NO: 73 (H-CDR1); the amino acid sequence of SEQ ID NO: 78 (H-CDR2); and the amino acid sequence of SEQ ID NO: 77 (H-CDR3), or
the amino acid sequence SEQ ID NO: 73 (H-CDR1); the amino acid sequence of SEQ ID NO: 79 (H-CDR2); and the amino acid sequence of SEQ ID NO: 77 (H-CDR3);
and
a light chain variable region comprising any one of:
the amino acid sequence of SEQ ID NO: 30 (L-CDR1); the amino acid sequence of SEQ ID NO: 31 (L-CDR2); and the amino acid sequence of SEQ ID NO: 32 (L-CDR3),
the amino acid sequence of SEQ ID NO: 164 (L-CDR1); the amino acid sequence of SEQ ID NO: 31 (L-CDR2); and the amino acid sequence of SEQ ID NO: 32 (L-CDR3),
the amino acid sequence of SEQ ID NO: 165 (L-CDR1); the amino acid sequence of SEQ ID NO: 166 (L-CDR2); and the amino acid sequence of SEQ ID NO: 32 (L-CDR3),
the amino acid sequence of SEQ ID NO: 165 (L-CDR1); the amino acid sequence of SEQ ID NO: 167 (L-CDR2); and the amino acid sequence of SEQ ID NO: 32 (L-CDR3).

In one embodiment, the CDRs of the anti-PD-1 antibody or antigen-binding fragment thereof are defined per the Chemical Computing Group (CCG) numbering.

In one embodiment, the present invention provides an anti-PD1 antibody or antigen-binding fragment thereof as set forth above, wherein the antibody or antigen-binding fragment thereof is a humanized antibody or antigen-binding fragment thereof.

In one embodiment, the present invention provides an anti-PD1 antibody or antigen-binding fragment thereof as set forth above, wherein the antibody or antigen-binding fragment thereof is selected from the group consisting of a monoclonal antibody, a Fab, a F(ab′)2, a Fv and an scFv.

In one embodiment, the present invention provides an anti-PD1 antibody or antigen-binding fragment thereof, wherein the antibody or antigen-binding fragment thereof comprises a heavy chain variable region and a light chain variable region comprising the amino acid sequences of SEQ ID NO: 108 and SEQ ID NO: 92, respectively; SEQ ID NO: 109 and SEQ ID NO: 93, respectively; SEQ ID NO: 110 and SEQ ID NO: 94, respectively; SEQ ID NO: 111 and SEQ ID NO: 95, respectively; SEQ ID NO: 112 and SEQ ID NO: 96, respectively; SEQ ID NO: 113 and SEQ ID NO: 97, respectively; SEQ ID NO: 114 and SEQ ID NO: 98, respectively; SEQ ID NO: 115 and SEQ ID NO: 99, respectively; SEQ ID NO: 116 and SEQ ID NO: 100, respectively; SEQ ID NO: 117 and SEQ ID NO: 101, respectively; SEQ ID NO: 118 and SEQ ID NO: 102, respectively; SEQ ID NO: 119 and SEQ ID NO: 103, respectively; SEQ ID NO: 120 and SEQ ID NO: 104, respectively; SEQ ID NO: 121 and SEQ ID NO: 105, respectively; SEQ ID NO: 122 and SEQ ID NO: 106, respectively; SEQ ID NO: 123 and SEQ ID NO: 107, respectively.

In one embodiment, the present invention provides an anti-PD1 antibody or antigen-binding fragment thereof, wherein the antibody or antigen-binding fragment thereof comprises a heavy chain variable region comprising the amino acid sequence of any one of SEQ NO: 131, SEQ NO: 133, SEQ NO: 135, SEQ NO: 137 or SEQ NO: 139 and a light chain variable region comprising the amino acid sequence of any one of SEQ NO: 125, SEQ NO: 127 or SEQ NO: 129.

In one embodiment, the present invention provides an anti-PD1 antibody or antigen-binding fragment thereof, wherein the antibody or antigen-binding fragment thereof comprises a heavy chain variable region and a light chain variable region comprising the amino acid sequences of SEQ ID NO: 131 and SEQ ID NO: 125, respectively.

In one embodiment, the present invention provides an anti-PD1 antibody or antigen-binding fragment thereof, wherein the antibody or antigen-binding fragment thereof comprises a heavy chain variable region and a light chain variable region comprising the amino acid sequences of SEQ ID NO: 133 and SEQ ID NO: 127, respectively.

In one embodiment, the present invention provides an anti-PD1 antibody or antigen-binding fragment thereof, wherein the antibody or antigen-binding fragment thereof comprises a heavy chain variable region and a light chain variable region comprising the amino acid sequences of SEQ ID NO: 135 and SEQ ID NO: 127, respectively.

In one embodiment, the present invention provides an anti-PD1 antibody or antigen-binding fragment thereof, wherein the antibody or antigen-binding fragment thereof comprises a heavy chain variable region and a light chain variable region comprising the amino acid sequences of SEQ ID NO: 137 and SEQ ID NO: 129, respectively.

In one embodiment, the present invention provides an anti-PD1 antibody or antigen-binding fragment thereof, wherein the antibody or antigen-binding fragment thereof comprises a heavy chain variable region and a light chain variable region comprising the amino acid sequences of SEQ ID NO: 139 and SEQ ID NO: 129, respectively.

In one embodiment, the present invention provides an anti-PD1 antibody or antigen-binding fragment thereof, wherein the antibody or antigen-binding fragment thereof comprises a heavy chain variable region and a light chain variable region having at least 90%, at least 95%, at least 98%, or at least 99% identity to the amino acid sequences of SEQ ID NO: 131 and SEQ ID NO: 125, respectively.

In one embodiment, the present invention provides an anti-PD1 antibody or antigen-binding fragment thereof, wherein the antibody or antigen-binding fragment thereof comprises a heavy chain variable region and a light chain variable region having at least 90%, at least 95%, at least 98%, or at least 99% identity to the amino acid sequences of SEQ ID NO: 133 and SEQ ID NO: 127, respectively.

In one embodiment, the present invention provides an anti-PD1 antibody or antigen-binding fragment thereof, wherein the antibody or antigen-binding fragment thereof comprises a heavy chain variable region and a light chain variable region having at least 90%, at least 95%, at least 98%, or at least 99% identity to the amino acid sequences of SEQ ID NO: 135 and SEQ ID NO: 127, respectively.

In one embodiment, the present invention provides an anti-PD1 antibody or antigen-binding fragment thereof, wherein the antibody or antigen-binding fragment thereof comprises a heavy chain variable region and a light chain variable region having at least 90%, at least 95%, at least 98%, or at least 99% identity to the amino acid sequences of SEQ ID NO: 137 and SEQ ID NO: 129, respectively.

In one embodiment, the present invention provides an anti-PD1 antibody or antigen-binding fragment thereof, wherein the antibody or antigen-binding fragment thereof comprises a heavy chain variable region and a light chain variable region having at least 90%, at least 95%, at least 98%, or at least 99% identity to the amino acid sequences of SEQ ID NO: 139 and SEQ ID NO: 129, respectively.

In one embodiment, the present invention provides an anti-PD1 antibody or antigen-binding fragment thereof as described above, wherein the antibody comprises a heavy chain constant region selected from the group consisting of IgG1, IgG2, IgG3, IgG4, IgM, IgA and IgE constant regions, for example human IgG1, IgG2, IgG3, IgG4, IgM, IgA or IgE.

In one embodiment, the present invention provides an anti-PD1 antibody as described above, wherein the heavy chain constant region is a heavy chain constant region of an IgG4 with a Ser228Pro mutation.

In one embodiment, the present invention provides an anti-PD1 antibody as described above, wherein the heavy chain constant region is a heavy chain constant region of an IgG1.

In one embodiment, the present invention provides an anti-PD1 antibody as described above, wherein the heavy chain constant region is a heavy chain constant region of an IgG1 with Leu234Ala and Leu235Ala mutations.

In one embodiment, the present invention provides an anti-PD1 antibody or antigen-binding fragment thereof as described above wherein the antibody or antigen-binding fragment thereof comprises a light chain constant region selected from the group consisting of kappa and lambda.

In one embodiment, the present invention provides an anti-PD1 antibody, wherein the antibody comprises a heavy chain and a light chain comprising the amino acid sequences of SEQ ID NO: 143 and SEQ ID NO: 141, respectively.

In one embodiment, the present invention provides an anti-PD1 antibody, wherein the antibody comprises a heavy chain and a light chain comprising the amino acid sequences of SEQ ID NO: 147 and SEQ ID NO: 145, respectively.

In one embodiment, the present invention provides an anti-PD1 antibody, wherein the antibody comprises a heavy chain and a light chain comprising the amino acid sequences of SEQ ID NO: 149 and SEQ ID NO: 145, respectively.

In one embodiment, the present invention provides an anti-PD1 antibody, wherein the antibody comprises a heavy chain and a light chain comprising the amino acid sequences of SEQ ID NO: 153 and SEQ ID NO: 151, respectively.

In one embodiment, the present invention provides an anti-PD1 antibody, wherein the antibody comprises a heavy chain and a light chain comprising the amino acid sequences of SEQ ID NO: 155 and SEQ ID NO: 151, respectively.

In one embodiment, the present invention provides an anti-PD1 antibody, wherein the antibody comprises a heavy chain and a light chain, wherein the amino acid sequence of said heavy chain consists of the amino acids of SEQ ID NO: 143 and the amino acid sequence of said light chain consists of the amino acids of SEQ ID NO:141.

In one embodiment, the present invention provides an anti-PD1 antibody, wherein the antibody comprises a heavy chain and a light chain, wherein the amino acid sequence of said heavy chain consists of the amino acids of SEQ ID NO: 147 and the amino acid sequence of said light chain consists of the amino acids of SEQ ID NO:145.

In one embodiment, the present invention provides an anti-PD1 antibody, wherein the antibody comprises a heavy chain and a light chain, wherein the amino acid sequence of said heavy chain consists of the amino acids of SEQ ID NO: 149 and the amino acid sequence of said light chain consists of the amino acids of SEQ ID NO:145.

In one embodiment, the present invention provides an anti-PD1 antibody, wherein the antibody comprises a heavy chain and a light chain, wherein the amino acid sequence of said heavy chain consists of the amino acids of SEQ ID NO: 153 and the amino acid sequence of said light chain consists of the amino acids of SEQ ID NO:151.

In one embodiment, the present invention provides an anti-PD1 antibody, wherein the antibody comprises a heavy chain and a light chain, wherein the amino acid sequence of said heavy chain consists of the amino acids of SEQ ID NO: 155 and the amino acid sequence of said light chain consists of the amino acids of SEQ ID NO:151.

In one embodiment, an anti-PD1 antibody or antigen-binding fragment thereof as described above is a monoclonal antibody or antigen-binding fragment thereof.

In one embodiment, an anti-PD1 antibody or antigen-binding fragment thereof as described above is a humanized antibody or antigen-binding fragment thereof.

In one embodiment, an anti-PD1 antibody or antigen-binding fragment thereof as described above is an agonist anti-PD1 antibody or antigen-binding fragment thereof.

In one embodiment, an anti-PD1 antibody or antigen-binding fragment thereof as described above binds to human PD-1 at high affinity, for example 20 nM of less, for example 10 nM or less, for example 5 nM of less.

In one embodiment, the present invention provides an anti-PD1 antibody or antigen-binding fragment thereof that competes for binding to PD-1 with an anti-PD-1 antibody or antigen-binding fragment thereof as described above. In one embodiment, the present invention provides an anti-PD-1 antibody or antigen-binding fragment thereof that competes for binding to PD-1 with Antibody A, Antibody B, Antibody C, Antibody D or Antibody E.

In one embodiment, the present invention provides a pharmaceutical composition comprising an anti-PD-1 antibody or antigen-binding fragment thereof as described above, and a pharmaceutically acceptable excipient.

In one embodiment, the present invention provides an anti-PD-1 antibody or antigen-binding fragment thereof as described above for use as a medicament.

In one embodiment, the present invention provides a method of treating a PD-1 pathway disorder comprising administering to a patient in need thereof a pharmaceutically effective amount of the anti-PD-1 antibody or antigen-binding fragment thereof as described above. In one embodiment, the present invention provides an anti-PD-1 antibody or antigen-binding fragment thereof as described above for use in treating a PD-1 pathway disorder. In one embodiment, the present invention provides the use of the anti-PD-1 antibody or antigen-binding fragment thereof as described above in manufacture of a medicament for treating a PD-1 pathway disorder.

In one embodiment, the present invention provides a method of modulating the interaction between PD-1 and PD-L1 in a human patient comprising administering to the human patient a composition comprising an anti-PD-1 antibody or the antigen-binding fragment as described above in an amount sufficient to activate the PD-1 pathway in the human patient. In one embodiment, the present invention provides an anti-PD-1 antibody or the antigen-binding fragment as described above for use in modulating the interaction between PD-1 and PD-L1 in a human patient. In one embodiment, the present invention provides the use of an anti-PD-1 antibody or the antigen-binding fragment as described above in the manufacture of a medicament for modulating the interaction between PD-1 and PD-L1 in a human patient.

In one embodiment, the present invention provides a method of attenuating PD-1 expressing T cell activity in a human patient comprising administering to the human patient a composition comprising an anti-PD-1 antibody or the antigen-binding fragment as described above in an amount sufficient to down-modulate an immune response in the human patient. In one embodiment, the present invention provides an anti-PD-1 antibody or the antigen-binding fragment as described above for use in attenuating PD-1 expressing T cell activity in a human patient. In one embodiment, the present invention provides the use of an anti-PD-1 antibody or the antigen-binding fragment as described above in the manufacture of a medicament for attenuating PD-1 expressing T cell activity in a human patient.

In one embodiment, in a method above, in an anti-PD-1 antibody or antigen-binding fragment thereof for use above, or in the use of an anti-PD-1 antibody or antigen-binding fragment thereof above, the disease is selected from the group consisting of systemic sclerosis (SSc), systemic lupus erythematosus, polymyositis, giant cell arteritis, psoriasis, psoriatic arthritis, ankylosing spondylitis and inflammatory bowel disease.

In one embodiment, in the method above, in the anti-PD-1 antibody or antigen-binding fragment thereof for use above, or in the use of the anti-PD-1 antibody or antigen-binding fragment thereof above, the antibody or antigen-binding fragment thereof is administered by a parenteral route, intravenous route or subcutaneous route of administration.

In one embodiment, the present invention provides an isolated polynucleotide encoding a heavy chain variable region amino and/or a light chain variable region as described above.

In one embodiment, the present invention provides an isolated polynucleotide encoding a heavy chain and/or a light chain as described above.

In one embodiment, the present invention provides an expression vector comprising a polynucleotide as described above.

In one embodiment, the present invention provides a host cell comprising an expression vector as described above. In one embodiment, the host cell is a mammalian cell.

In one embodiment, the present invention provides a method of manufacturing an antibody comprising the steps of:
culturing a host cell comprising an expression vector comprising an isolated polynucleotide encoding a heavy chain variable region as described above and an expression vector comprising polynucleotide encoding light chain variable region as described above under conditions that allow formation of an antibody, and
recovering said antibody.

In one embodiment, the present invention provides a method of manufacturing an antibody comprising the steps of:
culturing a host cell comprising an expression vector comprising an isolated polynucleotide encoding a heavy chain as described above and an expression vector comprising polynucleotide encoding light chain as described above under conditions that allow formation of an antibody; and
recovering said antibody.

In one embodiment, a method above further comprises the step of purifying the antibody. In one embodiment, a method above further comprises the step of formulating the antibody into a pharmaceutical composition.

In one embodiment, the present invention provides a multi-specific antibody comprising a first anti-PD-1 agonist antigen-binding site and a second antigen-binding site.

In one embodiment, the second antigen-binding site is an anti-CD48 binding site, an anti-CD-2 binding site, an anti-CD11a binding site or an anti-CD3 binding site.

In one embodiment, the first anti-PD-1 agonist antigen-binding site comprises a heavy chain variable region and a light chain variable region as described above.

In one embodiment, the multi-specific antibody is a bi-specific antibody.

### BRIEF DESCRIPTION OF THE DRAWINGS

FIG. 1: Selectivity of anti-PD-1 antibody to human PD-1 protein in a cell-based assay evaluated by flow cytometry. MFI stands for “mean fluorescence intensity”.

FIG. 2A, 2B: Competition binding assay of human PD-1-Fc binding to human PD-L1-Fc. Sensorgram depicting the binding curve of 25 nM PD-1-Fc to PD-L1-Fc amine coupled on the GLM chip surface (FIG. 2A). Sensorgrams of Antibody C, MK-3475, and PD1AB-6-4P (500 nM) pre-mixed with 25 nM PD-1-Fc binding to PD-L1-Fc amine coupled to the GLM chip surface (FIG. 2B).

FIG. 3A, 3B, 3C, 3D: Enhanced binding of PD-L1 to PD-1 in the presence of anti-PD-1 agonist antibody. PD-1 Biotin: PD-L1 interaction assay (FIG. 3A). CHO PD-1-PD-L1 Delphia-Eu TRF assay (FIGS. 3B and 3D). CHO PD-1: Biotin PD-L1 binding assay (FIG. 3C). In FIG. 3D, the individual data points are only depicted for Antibody C; the individual data points for the other antibodies are not depicted in FIG. 3D because of their close proximity to each other. POC stands for “percentage of control”.

FIG. 4A, 4B: T cell functional activity in the presence of anti-PD-1 agonist antibody or F(ab′)2 fragments derived from parental 723C2 agonist antibody (FIG. 4A) or parental agonist antibody 820C3 (FIG. 4B). POC stands for “percentage of control”.

FIG. 5: Induction of PD-1 activation by a MAb against CD48 to upon crosslinking.

FIG. 6: Labeling of human pan-T cell with CellTrace-Violet, activation with CD3 MAbs, and analysis of cell proliferation by dilution of the CellTrace.

FIG. 7A, 7B, 7C: Bispecific constructs (FIG. 7A). Binding of each arm to PD-1 or CD48 demonstrated by flow cytometry on Jurkat cells overexpressing PD-1 (FIG. 7B). Stimulation of human memory CD4+T (PD1+) cells with plate-bound anti-CD3e in presence of plate-bound PD-1/CD48 BsAbs or control antibodies (FIG. 7C). MFI stands for “mean fluorescence intensity”.

### DETAILED DESCRIPTION OF THE INVENTION

The present invention addresses the need for treatments for immune and inflammatory disorders, in particular immune and inflammatory disorders controlled by the PD-1/PD-L1 and/or PD-L2 system. To address this need, the present invention provides anti-PD-1 antibodies, which do not block the interaction between PD-1 and PD-L1. In one aspect, the present invention provides antibodies, which enhance the interaction between PD-1 and PD-L1. In one aspect of the invention, the antibodies of the present invention activate the PD-1 signaling pathway. In one aspect, the antibodies of the present invention are anti-PD-1 agonist antibodies. PD-1 agonism restores immune balance by inhibiting expansion and effector function of auto-reactive T cells in human diseases where PD-1 is expressed but may not be engaged, or optimally engaged by its ligands. In one aspect, the antibodies of the present invention are useful in treating in immune and inflammatory disorders and transplant rejection. For example, the antibodies of the present invention are useful for the treatment and/or prevention of diseases or disorders that can be alleviated by modulating the interaction between PD-1 and PD-L1, in particular by activating the PD-1 pathway. In one aspect, antibodies of the present invention are useful in treating and/or preventing systemic sclerosis (SSc), systemic lupus erythematosus, polymyositis, giant cell arteritis, psoriasis, psoriatic arthritis, ankylosing spondylitis or inflammatory bowel disease.

In one aspect, the present invention provides an anti-PD-1 antibody, in particular a monoclonal anti-PD-1 antibody, for example a humanized monoclonal anti-PD-1 antibody, having one or more of the properties described herein below. In one aspect, an anti-PD-1 antibody of the present invention binds to purified recombinant human PD-1 at high affinity, for example 20 nM or less, for example 10 nM or less, for example 5 nM of less. In one aspect, an anti-PD-1 antibody of the present invention binds to purified recombinant cynomologus PD-1 at an affinity of 50 nM or less. In one aspect, an anti-PD-1 antibody of the present invention selectively binds to PD-1, in particular human PD-1. In one aspect, an antibody of the present invention does not bind to mouse, rat, or rabbit PD-1. In one aspect, an anti-PD-1 antibody of the present invention does not block the binding of PD-L1 to PD-1. In one aspect, an anti-PD-1 antibody of the present invention enhances the binding of PD-L1 to PD-1. In one aspect, an anti-PD-1 antibody of the present invention attenuates T cell activity in several functional cell assays as shown herein below, for example by inhibition of IFNγ production, inhibition of IL-17A production or inhibition of IL-21 production. In one aspect, an anti-PD-1 antibody of the present invention inhibits human cell accumulation in a mouse model and reduces the levels of human inflammatory cytokines in the mouse model. In one aspect, an anti-PD-1 antibody of the present invention has favorable pharmacokinetic properties. In one aspect, an anti-PD-1 antibody of the present invention has favorable biophysical properties, for example yield, quality, stability or solubility. These properties are for example shown in the Examples herein below.

The generalized structure of antibodies or immunoglobulin is well known to those of skill in the art, these molecules are heterotetrameric glycoproteins, typically of about 150,000 daltons, composed of two identical light (L) chains and two identical heavy (H) chains. Each light chain is covalently linked to a heavy chain by one disulfide bond to form a heterodimer, and the heterotrimeric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (V_{H}=variable heavy chain), followed by three or four constant domains (C_{H1}, C_{H2}, C_{H3}, and C_{H4}), as well as a hinge region between C_{H1} and C_{H2}. Each light chain has two domains, an amino-terminal variable domain (V_{L}=variable light chain) and a carboxy-terminal constant domain (CL). The V_{L} domain associates non-covalently with the V_{H} domain, whereas the CL domain is commonly covalently linked to the C_{H1} domain via a disulfide bond. Particular amino acid residues are believed to form an interface between the light and heavy chain variable domains (Chothia et al., 1985, J. Mol. Biol. 186:651-663, Vargas-Madrazo E, Paz-García E. J Mol Recognit. 2003; 16(3): 113-120). The variable domains are also referred herein as variable regions, and the constant domains as constant regions.

Certain domains within the variable domains differ extensively between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5th Ed. Public Health Service, National Institutes of Health, Bethesda, Md., whereas HVLs are structurally defined according to the three-dimensional structure of the variable domain, as described by Chothia and Lesk, 1987, J. Mol. Biol. 196:901-917. Where these two methods result in slightly different identifications of a CDR, the structural definition is preferred. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. An alternative definition of the CDRs is per the Chemical Computing Group (CCG) numbering (Almagro et al., Proteins 2011; 79:3050-3066 and Maier et al, Proteins 2014; 82:1599-1610). The CDR1, CDR2, CDR3 of the heavy and light chains therefore define the unique and functional properties specific for a given antibody.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely β-sheet configuration of the FRs brings the CDRs within each of the chains into close proximity to each other as well as to the CDRs from the other chain. The resulting conformation contributes to the antigen binding site (see Kabat et al., 1991, NIH Publ. No. 91-3242, Vol. I, pages 647-669), although not all CDR residues are necessarily directly involved in antigen binding.

FR residues and Ig constant domains are generally not directly involved in antigen binding, but contribute to antigen binding and/or mediate antibody effector function. Some FR residues are thought to have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody-dependent cellular cytotoxicity (ADCC), complement-dependent cytotoxicity (CDC) and antibody-dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM IgG and IgA are further divided into subclasses (isotypes), e.g., IgG₁, IgG₂, IgG₃, IgG₄, IgA₁, and IgA₂, respectively. The heavy chain constant domains that correspond to the different classes of immunoglobulins are called α, δ, ε, γ, and μ, respectively. The subunit structures and three-dimensional configurations of the classes of native immunoglobulins are well known.

The terms, “antibody”, “anti-PD-1 antibody”, “humanized anti-PD-1 antibody”, and “variant humanized anti-PD-1 antibody” are used herein in the broadest sense and specifically encompass monoclonal antibodies (including full length monoclonal antibodies), multispecific antibodies (e.g., bispecific antibodies), antibodies with minor modifications such as N- or C-terminal truncations and antibody fragments such as variable domains and other portions of antibodies that exhibit a desired biological activity, e.g., PD-1 binding.

The term “monoclonal antibody” (mAb) refers to an antibody of a population of substantially homogeneous antibodies; that is, the individual antibodies in that population are identical except for naturally occurring mutations or possible well-known alterations such as removal of C-terminal lysine from the antibody heavy chain or post-translational modifications such as amino acid isomerization or deamidation, methionine oxidation or asparagine or glutamine deamidation that may be present in minor amounts. Monoclonal antibodies are highly specific, being directed against a single antigenic determinant, an “epitope”. Therefore, the modifier “monoclonal” is indicative of a substantially homogeneous population of antibodies directed to the identical epitope and is not to be construed as requiring production of the antibody by any particular method. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352:624-628, and Marks et al., 1991, J. Mol. Biol. 222:581-597.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81:6851-6855).

The terms “antibody fragment”, “antigen binding fragment”, “anti-PD-1 antibody fragment”, “humanized anti-PD-1 antibody fragment”, “variant humanized anti-PD-1 antibody fragment” refer to a portion of a full length anti-PD-1 antibody, in which a variable region or a functional capability is retained, for example, specific PD-1 epitope binding. Examples of antibody fragments include, but are not limited to, a Fab, Fab′, F(ab′)2, Fd, Fv, scFv and scFv-Fc fragment, a diabody, a linear antibody, a single-chain antibody, a minibody, a diabody formed from antibody fragments, and multispecific antibodies formed from antibody fragments.

Antibody fragments can be obtained for example by treating full-length antibodies treated with enzymes such as papain or pepsin to generate useful antibody fragments. Papain digestion is used to produce two identical antigen-binding antibody fragments called “Fab” fragments, each with a single antigen-binding site, and a residual “Fc” fragment. The Fab fragment also contains the constant domain of the light chain and the CHI domain of the heavy chain. Pepsin treatment yields a F(ab′)2 fragment that has two antigen-binding sites and is still capable of cross-linking antigen.

Another example of antibody fragments according to the invention are Fab′ fragments. Fab′ fragments differ from Fab fragments by the presence of additional residues including one or more cysteines from the antibody hinge region at the C-terminus of the C_{H1} domain. F(ab′)₂ antibody fragments are pairs of Fab′ fragments linked by cysteine residues in the hinge region. Other chemical couplings of antibody fragments are also known.

A “Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the V_{H}-V_{L} dimer. Collectively, the six CDRs confer antigen-binding specificity to the antibody.

Antibody fragments may also include “Single-chain Fv” or “scFv” fragments. A “single-chain Fv” or “scFv” antibody fragment is a single chain Fv variant comprising the V_{H} and V_{L} domains of an antibody where the domains are present in a single polypeptide chain. The single chain Fv is capable of recognizing and binding antigen. The scFv polypeptide may optionally also contain a polypeptide linker positioned between the V_{H} and V_{L} domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

Antibody fragments may also form tandem Fd segments, which comprise a pair of tandem Fd segments (V_{H}-C_{H1}-V_{H}-C_{H1}) to form a pair of antigen binding regions. These “linear antibodies” can be bispecific or monospecific as described in, for example, Zapata et al. 1995, Protein Eng. 8(10): 1057-1062.

In one aspect, an anti-PD-1 antibody of the invention is a humanized antibody or antibody fragment. A humanized antibody or a humanized antibody fragment is a specific type of chimeric antibody which includes an immunoglobulin amino acid sequence variant, or fragment thereof, which is capable of binding to a predetermined antigen and which, comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, a humanized antibody includes at least the CDRs or HVLs of a non-human antibody, inserted between the FRs of a human heavy or light chain variable domain. Methods of humanization of antibodies are for example described by Almagro et al., (2008) Frontiers in Bioscience 13, 1619-1633, or in WO12092374 A2.

The present invention describes specific humanized anti-PD-1 antibodies which contain CDRs derived from the mouse lead 723C2 inserted between the FRs of human germline sequence heavy and light chain variable domains. Additionally, a cysteine in the heavy chain CDR3 of mouse lead 723C2 was replaced with a tyrosine in the humanized anti-PD-1 antibodies derived from mouse lead 723C2 (“DC” to “DY”).

In one aspect, a humanized anti-PD-1 antibody comprises substantially all of at least one, and typically two, variable domains (such as contained, for example, in Fab, Fab′, F(ab′)2, Fabc, and Fv fragments) in which all, or substantially all, of the CDRs correspond to those of a non-human immunoglobulin, and specifically herein, the CDRs are murine sequences of the mouse lead 723C2 and the FRs are those of a human immunoglobulin consensus or germline sequence. In another aspect, a humanized anti-PD-1 antibody also includes at least a portion of an immunoglobulin Fc region, typically that of a human immunoglobulin. Ordinarily, the antibody will contain both the light chain as well as at least the variable domain of a heavy chain. The antibody also may include one or more of the C_{H1}, hinge, C_{H2}, C_{H3}, and/or C_{H4} regions of the heavy chain, as appropriate.

A humanized anti-PD-1 antibody according to the invention can be selected from any class of immunoglobulins, including IgM, IgG, IgD, IgA and IgE, and any isotype, including IgG₁, IgG₂, IgG₃, IgG₄, IgA₁ and IgA₂. For example, the constant domain can be a complement fixing constant domain where it is desired that the humanized antibody exhibits cytotoxic activity, and the isotype is typically IgG₁. Where such cytotoxic activity is not desirable, the constant domain may be of another isotype, e.g., IgG₂. An alternative humanized anti-PD-1 antibody can comprise sequences from more than one immunoglobulin class or isotype, and selecting particular constant domains to optimize desired effector functions is within the ordinary skill in the art.

In one aspect, the constant domain of an antibody of the present invention is IgG4Pro, which has one replacement mutation (Ser228Pro) that prevents Fab-arm exchanging. This Ser to Pro mutation is in the hinge region of the IgG4 backbone and is commonly known as Ser228Pro, although its position in the heavy chain may vary by a few amino acids, for example depending on the length of the variable region and/or difference of the hinge length between IgG1 and IgG4. The Ser to Pro mutation in the hinge region (Cys-Pro-Ser-Cys-Pro) is referred herein as “Ser228Pro”, independently of its position in the heavy chain. In another aspect, the constant domain of an antibody of the present invention is IgG1KO, which has two mutations in the hinge region, Leu234Ala and Leu235Ala, to reduce effector function (ADCC).

The FRs and CDRs, or HVLs, of a humanized anti-PD-1 antibody need not correspond precisely to the parental sequences. For example, one or more residues in the import CDR, or HVL, or the consensus or germline FR sequence may be altered (e.g., mutagenized) by substitution, insertion or deletion such that the resulting amino acid residue is no longer identical to the original residue in the corresponding position in either parental sequence but the antibody nevertheless retains the function of binding to PD-1. Such alteration typically will not be extensive and will be conservative alterations. Usually, at least 75% of the humanized antibody residues will correspond to those of the parental consensus or germline FR and import CDR sequences, more often at least 90%, and most frequently greater than 95%, or greater than 98% or greater than 99%.

Immunoglobulin residues that affect the interface between heavy and light chain variable regions (“the V_{L}-V_{H} interface”) are those that affect the proximity or orientation of the two chains with respect to one another. Certain residues that may be involved in interchain interactions include V_{L} residues 34, 36, 38, 44, 46, 87, 89, 91, 96, and 98 and V_{H} residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). U.S. Pat. No. 6,407,213 also discusses that residues such as V_{L} residues 43 and 85, and V_{H} residues 43 and 60 also may be involved in this interaction. While these residues are indicated for human IgG only, they are applicable across species. Important antibody residues that are reasonably expected to be involved in interchain interactions are selected for substitution into the consensus sequence.

The terms “consensus sequence” and “consensus antibody” refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Kabat et al., 1991, Sequences of Proteins of Immunological Interest, 5th Ed. Public Health Service, National Institutes of Health, Bethesda, Md., and variants thereof. The FRs of heavy and light chain consensus sequences, and variants thereof, provide useful sequences for the preparation of humanized anti-PD-1 antibodies. See, for example, U.S. Pat. Nos. 6,037,454 and 6,054,297.

Human germline sequences are found naturally in human population. A combination of those germline genes generates antibody diversity. Germline antibody sequences for the light chain of the antibody come from conserved human germline kappa or lambda v-genes and j-genes. Similarly the heavy chain sequences come from germline v-, d- and j-genes (LeFranc, M-P, and LeFranc, G, “The Immunoglobulin Facts Book” Academic Press, 2001).

An “isolated” antibody is one that has been identified and separated and/or recovered from a component of its natural environment. Contaminant components of the antibody's natural environment are those materials that may interfere with diagnostic or therapeutic uses of the antibody, and can be enzymes, hormones, or other proteinaceous or nonproteinaceous solutes. In one aspect, the antibody will be purified to at least greater than 95% isolation by weight of antibody, for example purified to at least greater than 95%, 96%, 97%, 98%, or 99%.

An isolated antibody includes an antibody in situ within recombinant cells in which it is produced, since at least one component of the antibody's natural environment will not be present. Ordinarily however, an isolated antibody will be prepared by at least one purification step in which the recombinant cellular material is removed.

The term “antibody performance” as used according to the invention refers to factors/properties that contribute to antibody recognition of antigen or the effectiveness of an antibody in vivo. Changes in the amino acid sequence of an antibody can affect antibody properties such as folding, and can influence physical factors such as initial rate of antibody binding to antigen (ka), dissociation constant of the antibody from antigen (k_{d}), affinity constant of the antibody for the antigen (Kd), conformation of the antibody, protein stability, and half-life of the antibody.

The term “agonist antibody” or “agonistic antibody” as used according to the invention refers to an antibody, which upon binding to PD-1, induces at least one biological activity that is induced by PD-1 ligand PD-L1. In one aspect, the induction is statistically significant when compared to the induction in the absence of the agonist antibody. In one aspect, the antibody is an agonist antibody when the at least one biological activity is induced by at least about 20%, 30%, 40%, 45%, 50%, 55%, 60%, 65%, 70%, 75%, 80%, 85%, 90%, 95%, or 100% greater than in the absence of the agonist antibody, for example when measured as in one of the Examples described herein below. In some embodiments, an “agonist antibody” enhances the interaction between PD-1 and PD-L1. Exemplary assays for detecting PD-1 agonist properties are described herein or are known in the art.

“Multispecific” refers to a protein, such as an antibody, that specifically binds two or more distinct antigens or two or more distinct epitopes within the same antigen.

“Bispecific” refers to a protein, such as an antibody, that specifically binds two distinct antigens or two distinct epitopes within the same antigen.

In some embodiments, the antibody that specifically binds PD-1 or the antigen-binding fragment thereof of the invention is a bispecific antibody. In some embodiments, the antibody or the antigen-binding fragment thereof of the invention is a multispecific antibody. The monospecific antibodies that specifically bind PD-1 provided herein may be engineered into bispecific antibodies, which are also encompassed within the scope of the invention.

Full-length bispecific antibodies may be generated for example using Fab arm exchange (e.g., half-molecule exchange, exchanging one heavy chain-light chain pair) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond

Bispecific antibodies may also be generated using designs such as the Triomab/Quadroma (Trion Pharma/Fresenius Biotech), Knob-in-Hole (Genentech), CrossMAbs (Roche) and the electrostatically-induced CH3 interaction (Chugai, Amgen, NovoNordisk, Oncomed), the LUZ-Y (Genentech), the Strand Exchange Engineered Domain body (SEEDbody) (EMD Serono), the Biclonic (Merus) and as DuoBody® Products (Genmab A/S).

For example, a bispecific PD-1/CD2, a bispecific PD-1/CD48, a bispecific PD-1/CD11a or a PD-1/CD3 antibody can be generated using the VH/VL domains of the PD-1 antibodies described herein or any VH/VL regions of published anti-PD-1 agonist antibodies and any VH/VL regions of published anti-CD2, anti-CD48, anti-CD11a or anti-CD3 antibodies, respectively.

Another embodiment of the invention is a bispecific antibody comprising a first domain that binds PD-1 and a second domain that binds CD2, CD48, CD11a or CD3.

As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences that are compared are the same length after gaps are introduced within the sequences, as appropriate (e.g., excluding additional sequence extending beyond the sequences being compared). For example, when variable region sequences are compared, the leader and/or constant domain sequences are not considered. For sequence comparisons between two sequences, a “corresponding” CDR refers to a CDR in the same location in both sequences (e.g., CDR-H1 of each sequence).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (ld.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

As used herein, the expressions “cell”, “cell line”, and “cell culture” are used interchangeably and all such designations include the progeny thereof. Thus, “transformants” and “transformed cells” include the primary subject cell and cultures derived therefrom without regard for the number of transfers, which may for example have been transfected with one or more expression vectors encoding one or more amino acids sequences of an antibody or antigen binding fragment thereof of the present invention.

The term “mammal” for purposes of treatment according to the invention refers to any animal classified as a mammal, including humans, domesticated and farm animals, and zoo, sports, or pet animals, such as dogs, horses, cats, cows, and the like. Preferably, the mammal is human.

A “disorder”, as used herein, is any condition that would benefit from treatment with an anti-PD-1 antibody described herein, in particular a humanized anti-PD-1 antibody described herein. This includes chronic and acute disorders or diseases including those pathological conditions that predispose the mammal to the disorder in question.

As used herein, the term “PD-1 pathway disorder” or “PD-1 pathway disease” refers to a condition, which can be alleviated by modulating the interaction between PD-1 and PD-L1, in particular by activating the PD-1 pathway. A “PD-1 pathway disorder” or “PD-1 pathway disease” includes T-cell associated diseases where PD-1 is expressed, A “PD-1 pathway disorder” or “PD-1 pathway disease” also includes conditions characterized by activated auto-reactive T cells that express PD-1 and are drivers of chronic inflammation and autoimmune disease and in which attenuation of PD-1 expressing T cell activity and/or downmodulation of immune response is desired. Examples of PD-1 pathway disorders include diseases or disorders such as systemic sclerosis (SSc), systemic lupus erythematosus, polymyositis, giant cell arteritis, psoriasis, psoriatic arthritis, ankylosing spondylitis and inflammatory bowel disease.

The term “specifically binds,” or the like, means that an anti-PD-1 antibody or antigen-binding fragment thereof forms a complex with an antigen that is relatively stable under physiologic conditions. Methods for determining whether two molecules specifically bind are described herein or a known in the art and include, for example, equilibrium dialysis, surface plasmon resonance, and the like. In one embodiment, specific binding is characterized by a K_{D} of about 1×10⁻⁷ M (100 nM) or less according to the Affinity Binding method described in the Examples section herein. In another embodiment, specific binding is characterized by a Kp of about 5×10⁻⁸ M (50 nM) or less according to the Affinity Binding method described in the Examples section herein. In another embodiment, specific binding is characterized by a K_{D} of about 1×10⁻⁸ M (10 nM) or less according to the Affinity Binding method described in the Examples section herein. In another embodiment, specific binding is characterized by a K_{D} of about 5×10⁻⁹ M (5 nM) or less according to the Affinity Binding method described in the Examples section herein. An isolated antibody that specifically binds human PD-1 may, however, have cross-reactivity to other antigens, such as PD-1 molecules from other species. Moreover, an isolated antibody may be substantially free of other cellular material and/or chemicals.

The term “subcutaneous administration” refers to introduction of a drug, for example an anti-PD-1 antibody or antigen-binding fragment thereof of the invention, under the skin of an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug, for example an anti-PD-1 antibody or antigen-binding fragment thereof of the invention, under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes; in another aspect, less than 5 minutes, and in still another aspect, less than 60 seconds. In yet even another aspect, administration is within a pocket between the skin and underlying tissue, where the pocket may be created by pinching or drawing the skin up and away from underlying tissue. For example, “subcutaneous bolus” refers to the administration of an anti-PD-1 antibody or antigen-binding fragment thereof of the invention to a human patient in less than approximately 15 minutes; in another aspect, less than 5 minutes, and in still another aspect, less than 60 seconds

The term “therapeutically effective amount” is used to refer to an amount of an anti-PD-1 antibody or antigen-binding fragment thereof that relieves or ameliorates one or more of the symptoms of the disorder being treated. In doing so, it is that amount that has a beneficial patient outcome. Efficacy can be measured in conventional ways, depending on the condition to be treated.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an anti-PD-1 antibody or antigen-binding fragment thereof prior to or following the onset of a symptom of a disease or disorder thereby preventing or removing one or more signs of the disease or disorder. As another example, the term includes the administration of an anti-PD-1 antibody or antigen-binding fragment thereof after clinical manifestation of the disease to combat the symptoms of the disease. Further, administration of an anti-PD-1 antibody or antigen-binding fragment thereof after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the anti-PD-1 antibody composition or antigen-binding fragment thereof, the result should be considered an effective treatment of the underlying disorder regardless of whether all the symptoms of the disorder are alleviated or not.

The term “package insert” is used to refer to instructions customarily included in commercial packages of therapeutic products, that contain information about the indications, usage, administration, contraindications and/or warnings concerning the use of such therapeutic products.

### Antibodies

Described and disclosed herein are anti-PD-1 antibodies, in particular humanized anti-PD-1 antibodies, as well as compositions and articles of manufacture comprising anti-PD-1 antibodies of the present invention. Also described are antigen-binding fragments of an anti-PD-1 antibody. The anti-PD-1 antibodies and antigen-binding fragments thereof can be used in the treatment of a variety of diseases or disorders, in particular diseases or disorders characterized by activated auto-reactive T cells that express PD-1 and are drivers of chronic inflammation and autoimmune disease. An anti-PD-1 antibody and an antigen-binding fragment thereof each include at least a portion that specifically recognizes a PD-1 epitope. In one aspect, the anti-PD-1 antibodies of the present invention and antigen-binding fragments thereof are agonist anti-PD-1 antibodies and antigen-binding fragment thereof.

The generation of anti-PD-1 antibodies according to the invention and their characterization is described in the Examples. In an initial characterization, the anti-PD-1 chimeric lead 723C2 was selected based on its superior antibody performance, as for example described in the Examples below. A library of variants was generated by placing the CDRs of the chimeric lead into FRs of the human consensus heavy and light chain variable domains and furthermore by engineering the FRs with different alterations. Additionally, a cysteine in the heavy chain CDR3 of mouse lead 723C2 was replaced with a tyrosine in the humanized anti-PD-1 antibodies derived from mouse lead 723C2 (“DC” to “DY”). The change from “DC” to “DY” did not have an impact on the pharmacological properties of the antibodies. The process for the production of humanized antibodies is described in the Examples.

The amino acid sequences of variable regions of representative mouse leads are shown in Tables 1 and 2. The CDR regions of these mouse leads and the CDR regions of engineered variants of lead 723C2 are shown in Tables 3 and 4.

TABLE 1
Anti-PD-1 Mouse Leads-VK Sequences

| | |
|---|---|
| 306E6VK | EIVMTQAAFSNPVTLGTSASISCRSSKSLLHRNGITYLYWYLQKPGQSP |
| | QLLIYEMSNLASGVPDRFSSSGSGTDFTLRISRVEAEDVGVYYCGQNLE |
| | FPLTFGAGTKLELK (SEQ ID NO: 92) |
| 307A3VK | EIVMTQAAFSNPVTLGTSASISCRSSKSLLHRNGITYLYWYLQKPGQSP |
| | QLLIYEMSNLASGVPDRFSGSGSGTDFTLRISRVEAEDVGVYYCGQNLE |
| | FPLTFGAGTKLELK (SEQ ID NO: 93) |
| 313C12VK | DIVMTQSQKFMSTTVGDRVSITCKASQNVGTAVAWYQQKPGQSPKLLIY |
| | SVSNRYTGVPDRFTGSGSGTDFTLTISNMQSEDLADYFCQQYSSYPFTF |
| | GAGTKLELK (SEQ ID NO: 94) |
| 414A12VK | DILMTQSPSSMSVSLGDTVSITCHASQGINNNIGWLQQKPGKSFKGLIY |
| | HKSNLEDGVPSRFSGSGSGADYSLTISSLESEDFADYYCVQYAQFPYTF |
| | GGGTKLEIK (SEQ ID NO: 95) |
| 502H1VK | DIVMTQAAFYNPVTLGTSASISCRSSKSLLHRNGITYLYWYLQKPGQSP |
| | QLLIYQMSNLASGVPDRFSSSGSGADFTLRISRVEAEDVGVYYCAQNLE |
| | LPLTFGAGTKLELK (SEQ ID NO: 96) |
| 701C1VK | DIVMSQSPSSLAVSVGEKVTMTCKSSQSLLYSSNQKNYLAWYQQKPGQS |
| | PKLLIYWASTRESGVPDRFTGSGSGTDFTLTISSVKAEDLAVYYCHQYY |
| | SSPLTFGAGTKLELK (SEQ ID NO: 97) |
| 701E9VK | DIQMTQSPSSLSASLGERVSLTCRASQEISGYLSWLQQKPDGTIKRLIY |
| | AASTLDSGVPKRFSGSRSGSDYSLTISSLESEDFADYYCLQYASYPLTF |
| | GAGTKLELK (SEQ ID NO: 98) |
| 703D10VK | QIVLTQSPGIMSASPGEKVTITCSANSSVSFMHWFQQKPGTSPKIWIYS |
| | TSSLASGVPARFSGSGSGTSYSLTISRMEAEDAATYYCQQRSSYPLTFG |
| | AGTKLELK (SEQ ID NO: 99) |
| 708E4VK | DIVMTQSQKFLSTSVGDRVRVTCKASQNVVTYVAWYQQKPGQSPKSLIY |
| | SASYRYSGVPDRFTGSGSGTYFTLTINNVQFEDLAEYFCQQYHSYPYTF |
| | GGGTKLEIK (SEQ ID NO: 100) |
| 709A6VK | DIVLTQSPASLAVSLGQRATISCRASESVDIYGISFLHWYQQKPGQPPK |
| | HLIYRASNLDSGIPARFSGSGSRTDFTLTINPVETDDVATYYCQQSNKD |
| | PLTFGAGTKLELK (SEQ ID NO: 101) |
| 718C2VK | DIVMSQSPSSLTVSVGEKVTMSCKSSQSLLYSSNQKIYLAWFQQKPGQS |
| | PKLLIYWASTRESGVPDRFTGSGSGTDFTLTISSVKAEDLAVYYCQQYY |
| | NSPLTFGAGTKLELK (SEQ ID NO: 102) |
| 723C2VK | EIVLTQSPTTMAASPGEKITITCSASSSISSDYLHWYQQKPGFSPELLI |
| | YRTSNLASGVPARFSGSGSGTSYSLTIGSMEAEDVATYYCQQGTSLPRA |
| | FGGGTKLEIK (SEQ ID NO: 103) |
| 803E6VK | DIVMTQSPSSLTVTAGEKVTMSCKSSQSLLHSGNQKNYMTWYQQKPGQP |
| | PKLLIYWASTRESGVPDRFTGSGSGTDFTLTISSVQAEDLAVYYCQNDY |
| | SYPLTFGAGTKLELK (SEQ ID NO: 104) |
| 811G3VK | DIQMTQSPSSLSASPGERVSLTCRASQEISGYLSWLQQKPDGTIKRLIY |
| | VASTLDSGVPKRFSGSRSGSDYSLTISSLESEDFADYYCLQYANYPYTF |
| | GGGTKLEIK |
| | (SEQ ID NO: 105) |
| 814E10VK | DIQMTQTTSSLSASLGDRVTISCSASQDIINYLNWYQQKPDGTVKLLIY |
| | STSSLHSGVSLRFSGSGSGTDYSLTISNLEPEDVATYYCHQYSQLPYTF |
| | GGGTKLEIK (SEQ ID NO: 106) |
| 820C3VK | DIQMTQTTSSLSASLGDRVTISCSASQDIFNYLNWYQQKPDGTVKLLIY |
| | YTSSLHSGVPSRFSGSGSGTDFSLTISNLEPEDIATYYCQQYSNLPYTF |
| | GGGTKLEIK (SEQ ID NO: 107) |

TABLE 2
Anti-PD-1 Mouse Leads-VH Sequences

| | |
|---|---|
| 306E6VH | EVQLQQSGPELVKPGSSVKISCKASGYTFTDYYVNWVKQSHGKSLEWFGD |
| | IHPNSGDTTYNQKFKDKATLTVDKSSSTAYMELRSLTSEDSAVYYCARRR |
| | YDYDGFAYWGQGTLVTVSA |
| | (SEQ ID NO: 108) |
| 307A3VH | EVQLQQSGPELVKPGASVKISCKASGYTFTDYYVNWVKQSHGKSLEWFGD |
| | IHPNNGGITYNQKFKGKASLTVDKSSSTAYMELRSLTSEDSAVYYCARRR |
| | YDYDGFAYWGQGTLVTVSA |
| | (SEQ ID NO: 109) |
| 313C12VH | EVQLVESGGGLVKPGGSLKLSCAASGFTFSDYGMHWVRQTPEKGLEWIAY |
| | INSDSNTIYYADTVKGRFTISRDNARKTLYLQMTSLRSEDTAMYYCSPLV |
| | APDYWGQGTTLTVSS |
| | (SEQ ID NO: 110) |
| 414A12VH | QVQLQQPGAELVKPGASVKLSCKASGHTFTSNWIHWVKQRPGQGLEWIGE |
| | IDPSDSYTYYNQKFKGKATLTVDKSSSTAYMQLSSLTSEDSAVYSCACPG |
| | RNSNFAYWGQGTTLTVSS |
| | (SEQ ID NO: 111) |
| 502H1VH | QVTLKESGPGILQPSQTLSLTCSFSGFSLSTSGMGVTWIRKPSGQGLEWL |
| | AHIFWDGDKRYNPSLKSRLTISKDSSSNQVFLMITGVGTADAATYYCARY |
| | YYFDYGYAIDYWGQGTSVTVSS |
| | (SEQ ID NO: 112) |
| 701C1VH | EVQLQQSGPELVKPGASVKMSCKASGYTFTSYVIHWVKQKPGQGLEWIGY |
| | IDPSNDDTKYNEKFKGKATLTSDKSSSTAYMELSSLTSEDSAVYYCAREA |
| | YYGGLYGMDYWGQGTSVTVSS |
| | (SEQ ID NO: 113) |
| 701E9VH | QVQLQQSGPELVKPGASVKLSCKASGYTFIDYTIHWVKQSPGQGLEWIGW |
| | IFPGSTNDTKYNDKFKGKATMTADKSSSTAYMQLSSLTSEDSAVYFCARY |
| | RTDFDYWGQGTTLTVSS |
| | (SEQ ID NO: 114) |
| 703D10VH | QVQLQQPGAELVKPGASVKLSCKASGYSFTSYWMHWVRQRPGQGLEWIGD |
| | IDPSNSYAYHSQKFKGKATLTVDKSSSTAYMQLSSLTSEDSAVYFCARAD |
| | GTSHWYFDVWGAGTAVTVSS |
| | (SEQ ID NO: 115) |
| 708E4VH | QIQLQQSGPELVKPGASVKISCKASGYTFTDYYLNWVKQRPGHGLEWIGW |
| | IYPGSSDTKHNENFKGKATLTVDTYSSTAYMQLGSLTSEDSAVYFCTRYS |
| | NFFFDYWGQGTTLTVSS |
| | (SEQ ID NO: 116) |
| 709A6VH | QVTLKESGPGILQPSQTLSLTCSFSGFSLSTSGMGVSWIRQPSGKGLEWL |
| | THIYWDDDKRYNPSLKSRLTISKDTSRNQVFLEITSVDTADTATYFCARS |
| | SQGLYSSYDYWGQGTTLTVSS |
| | (SEQ ID NO: 117) |
| 718C2VH | EVQLQQSGPELVKPGASVKMSCKASGYTFTSYVMHWVRQKPGQGLEWIGY |
| | IDPDNDGTKYNEKFKGKATLTSDKSSSTAYMELSSLTSEDSAVYYCAREA |
| | YYGGLYGMDYWGQGSSVTVSS |
| | (SEQ ID NO: 118) |
| 723C2VH | EVQLVESGGGLVQPGGSLKLSCAASGFTFSDYYMSWVRQTPEKRLEWVAY |
| | ISSGGGSSYYPDSVKGRFTISRDNTKNTLYLQMSSLKSEDTAVYYCARLP |
| | HYFAMDCWGQGTSVTVSS |
| | (SEQ ID NO: 119) |
| 803E6VH | EVKLVESEGGLVQPGSSMKLSCTASGFTFSDYYMAWVRQVPEKGLEWVAN |
| | INYDGFNTYYLDSLKSRFIISRDNAKNILYLQMSSLKSEDTATYYCARGG |
| | YWSLYFDYWGQGTTLTVSS |
| | (SEQ ID NO: 120) |
| 811G3VH | QIQLQQSGPEVVKPGASVKISCKASGYTFTDYYINWVKQRPGQGLEWIGW |
| | IYPGGGHTKYNEKFKGEATLTVDTSSRTAYMQLSSLTSEDSAVYFCARYS |
| | NYYFDFWGHGTTLTVSS |
| | (SEQ ID NO: 121) |
| 814E10VH | QIQLQQSGPELVKPGASVKISCKASGYTFTSYYIQWVKQRPGQGLEWIGW |
| | IYPGDGTTNYNENFKGKTTLTADKSSSTVYMLLSSLTSEDSAVYFCARYG |
| | LVPFDYWGQGTTLTVSS |
| | (SEQ ID NO: 122) |
| 820C3VH | QIQLQQSGPELVKTGASVKISCKASGNTFNSNYIQWVKQRPGQGLEWIGW |
| | IYPGDGSTNYSEKFKGKTTLTADKSSSTAYMLVSSLTSEDSAVYFCARYG |
| | PVPFDYWGQGTTLSVSS |
| | (SEQ ID NO: 123) |

The mouse light chain and heavy chain CDRs of the various mouse antibodies are shown in Table 3 and Table 4, respectively. Tables 3 and 4 also show three light chain CDRs and three heavy chains CDRs derived from the mouse antibody 723C2 through the humanization process.

TABLE 3
LIGHT CHAIN CDR sequences

| | L-CDR1 | L-CDR2 | L-CDR3 |
|---|---|---|---|
| 306E6 | RSSKSLLHRNGITYLY | EMSNLAS | GQNLEFPLT |
| | (SEQ ID NO: 1) | (SEQ ID NO: 2) | (SEQ ID NO: 3) |
| 307A3 | RSSKSLLHRNGITYLY | EMSNLAS | GQNLEFPLT |
| | (SEQ ID NO: 1) | (SEQ ID NO: 2) | (SEQ ID NO: 3) |
| 313C12 | KASQNVGTAVA | SVSNRYT | QQYSSYPFT |
| | (SEQ ID NO: 4) | (SEQ ID NO: 5) | (SEQ ID NO: 6) |
| 414A12 | HASQGINNNIG | HKSNLED | VQYAQFPYT |
| | (SEQ ID NO: 7) | (SEQ ID NO: 8) | (SEQ ID NO: 9) |
| 502H1 | RSSKSLLHRNGITYLY | QMSNLAS | AQNLELPLT |
| | (SEQ ID NO: 10) | (SEQ ID NO: 11) | (SEQ ID NO: 12) |
| 701C1 | KSSQSLLYSSNQKNYLA | WASTRES | HQYYSSPLT |
| | (SEQ ID NO: 13) | (SEQ ID NO: 14) | (SEQ ID NO: 15) |
| 701E9 | RASQEISGYLS | AASTLDS | LQYASYPLT |
| | (SEQ ID NO: 16) | (SEQ ID NO: 17) | (SEQ ID NO: 18) |
| 703D10 | SANSSVSFMH | STSSLAS | QQRSSYPLT |
| | (SEQ ID NO: 19) | (SEQ ID NO: 20) | (SEQ ID NO: 21) |
| 708E4 | KASQNVVTYVA | SASYRYS | QQYHSYPYT |
| | (SEQ ID NO: 22) | (SEQ ID NO: 23) | (SEQ ID NO: 24) |
| 709A6 | RASESVDIYGISFLH | RASNLDS | QQSNKDPLT |
| | (SEQ ID NO: 25) | (SEQ ID NO: 26) | (SEQ ID NO: 27) |
| 718C2 | KSSQSLLYSSNQKIYLA | WASTRES | QQYYNSPLT |
| | (SEQ ID NO: 28) | (SEQ ID NO: 14) | (SEQ ID NO: 29) |
| 723C2 | SASSSISSDYLH | RTSNLAS | QQGTSLPRA |
| | (SEQ ID NO: 30) | (SEQ ID NO: 31) | (SEQ ID NO: 32) |
| Hu_723C2- | SASQSISSDYLH | RTSNLAS | QQGTSLPRA |
| 463-60 | (SEQ ID NO: 164) | (SEQ ID NO: 31) | (SEQ ID NO: 32) |
| Hu_723C2- | QASQSISSDYLH | RTSNLET | QQGTSLPRA |
| 462-07 | (SEQ ID NO: 165) | (SEQ ID NO: 166) | (SEQ ID NO: 32) |
| Hu_723C2- | QASQSISSDYLH | RTSNLES | QQGTSLPRA |
| 462-08 | (SEQ ID NO: 165) | (SEQ ID NO: 167) | (SEQ ID NO: 32) |
| 803E6 | KSSQSLLHSGNQKNYMT | WASTRES | QNDYSYPLT |
| | (SEQ ID NO: 33) | (SEQ ID NO: 14) | (SEQ ID NO: 34) |
| 811G3 | RASQEISGYLS | VASTLDS | LQYANYPYT |
| | (SEQ ID NO: 16) | (SEQ ID NO: 35) | (SEQ ID NO: 36) |
| 814E10 | SASQDIINYLN | STSSLHS | HQYSQLPYT |
| | (SEQ ID NO: 37) | (SEQ ID NO: 38) | (SEQ ID NO: 39) |
| 820C3 | SASQDIFNYLN | YTSSLHS | QQYSNLPYT |
| | (SEQ ID NO: 40) | (SEQ ID NO: 41) | (SEQ ID NO: 42) |

TABLE 4
HEAVY CHAIN CDR sequences

| | H-CDR1 | H-CDR2 | H-CDR3 |
|---|---|---|---|
| 306E6 | GYTFTDYYVN | DIHPNSGDTTYNQKFKD | RRYDYDGFAY |
| | (SEQ ID NO: 43) | (SEQ ID NO: 44) | (SEQ ID NO: 45) |
| 307A3 | GYTFTDYYVN | DIHPNNGGITYNQKFKG | RRYDYDGFAY |
| | (SEQ ID NO: 43) | (SEQ ID NO: 46) | (SEQ ID NO: 45) |
| 313C12 | GFTFSDYGMH | YINSDSNTIYYADTVKG | LVAPDY |
| | (SEQ ID NO: 47) | (SEQ ID NO: 48) | (SEQ ID NO: 49) |
| 414A12 | GHTFTSNWIH | EIDPSDSYTYYNQKFKG | PGRNSNFAY |
| | (SEQ ID NO: 50) | (SEQ ID NO: 51) | (SEQ ID NO: 52) |
| 502H1 | GFSLSTSGMGVT | HIFWDGDKRYNPSLKS | YYYFDYGYAIDY |
| | (SEQ ID NO: 53) | (SEQ ID NO: 54) | (SEQ ID NO: 55) |
| 701C1 | GYTFTSYVIH | YIDPSNDDTKYNEKFKG | EAYYGGLYGMDY |
| | (SEQ ID NO: 56) | (SEQ ID NO: 57) | (SEQ ID NO: 58) |
| 701E9 | GYTFIDYTIH | WIFPGSTNDTKYNDKFKG | YRTDFDY |
| | (SEQ ID NO: 59) | (SEQ ID NO: 60) | (SEQ ID NO: 61) |
| 703D10 | GYSFTSYWMH | DIDPSNSYAYHSQKFKG | ADGTSHWYFDV |
| | (SEQ ID NO: 62) | (SEQ ID NO: 63) | (SEQ ID NO: 64) |
| 708E4 | GYTFTDYYLN | WIYPGSSDTKHNENFKG | YSNFFFDY |
| | (SEQ ID NO: 65) | (SEQ ID NO: 66) | (SEQ ID NO: 67) |
| 709A6 | GFSLSTSGMGVS | HIYWDDDKRYNPSLKS | SSQGLYSSYDY |
| | (SEQ ID NO: 68) | (SEQ ID NO: 69) | (SEQ ID NO: 70) |
| 718C2 | GYTFTSYVMH | YIDPDNDGTKYNEKFKG | EAYYGGLYGMDY |
| | (SEQ ID NO: 71) | (SEQ ID NO: 72) | (SEQ ID NO: 58) |
| 723C2 | GFTFSDYYMS | YISSGGGSSYYPDSVKG | LPHYFAMDC |
| | (SEQ ID NO: 73) | (SEQ ID NO: 74) | (SEQ ID NO: 75) |
| Hu_723C2- | GFTFSDYYMS | YISSGGGSKYYPDLVKG | LPHYFAMDY |
| 463-60 | (SEQ ID NO: 73) | (SEQ ID NO: 76) | (SEQ ID NO: 77) |
| Hu_723C2- | GFTFSDYYMS | YISSGGGSSYYPDAVKG | LPHYFAMDY |
| 461-47 | (SEQ ID NO: 73) | (SEQ ID NO: 78) | (SEQ ID NO: 77) |
| Hu_723C2- | GFTFSDYYMS | YISSGGGSSYYPDLVKG | LPHYFAMDY |
| 461-40 | (SEQ ID NO: 73) | (SEQ ID NO: 79) | (SEQ ID NO: 77) |
| 803E6 | GFTFSDYYMA | NINYDGFNTYYLDSLKS | GGYWSLYFDY |
| | (SEQ ID NO: 80) | (SEQ ID NO: 81) | (SEQ ID NO: 82) |
| 811G3 | GYTFTDYYIN | WIYPGGGHTKYNEKFKG | YSNYYFDF |
| | (SEQ ID NO: 83) | (SEQ ID NO: 84) | (SEQ ID NO: 85) |
| 814E10 | GYTFTSYYIQ | WIYPGDGTTNYNENFKG | YGLVPFDY |
| | (SEQ ID NO: 86) | (SEQ ID NO: 87) | (SEQ ID NO: 88) |
| 820C3 | GNTFNSNYIQ | WIYPGDGSTNYSEKFKG | YGPVPFDY |
| | (SEQ ID NO: 89) | (SEQ ID NO: 90) | (SEQ ID NO: 91) |

The CDRs listed above in Tables 3 and 4 are defined using the Chemical Computing Group (CCG) numbering are underlined (Almagro et al., Proteins 2011; 79:3050-3066 and Maier et al, Proteins 2014; 82:1599-1610).

A representative number of humanized light chain and heavy chain variable regions derived from mouse antibody 723C2 are provided and shown in Tables 5 and 6.

TABLE 5
Humanized 723C2-VK Sequences

| | |
|---|---|
| 723C2VK- | GATATCCAGATGACGCAGAGCCCAAGCAGCCTGAGCGCGTCCGTGGG |
| 463-60 | CGACCGCGTGACGATCACCTGTAGCGCGTCCCAGAGCATCAGCAGCG |
| | ACTATCTGCATTGGTATCAGCAGAAACCAGGTAAAGCCCCTAAACTG |
| | CTGATCTACCGGACCTCCAATCTGGCAAGCGGCGTGCCTAGCCGTTT |
| | CAGCGGTAGCGGCTCCGGTACCGACTTCACCTTTACTATCTCCAGCC |
| | TGCAGCCTGAAGACATCGCGACGTATTATTGTCAGCAGGGTACTAGC |
| | CTGCCTCGCGCCTTCGGCCAGGGGACCAAACTGGAAATCAAA |
| | (SEQ ID NO: 124) |
| | DIQMTQSPSSLSASVGDRVTITCSASQSISSDYLHWYQQKPGKAPKL |
| | LIYRTSNLASGVPSRFSGSGSGTDFTFTISSLQPEDIATYYCQQGTS |
| | LPRAFGQGTKLEIK |
| | (SEQ ID NO: 125) |
| 723C2VK- | GATATCCAGATGACGCAGAGCCCAAGCAGCCTGAGCGCGTCCGTGGG |
| 462-07 | CGACCGCGTGACGATCACCTGTCAGGCGTCCCAGAGCATCAGCAGCG |
| | ACTATCTGCATTGGTATCAGCAGAAACCAGGTAAAGCCCCTAAACTG |
| | CTGATCTACCGGACCTCCAATCTGGAAACCGGCGTGCCTAGCCGTTT |
| | CAGCGGTAGCGGCTCCGGTACCGACTTCACCTTTACTATCTCCAGCC |
| | TGCAGCCTGAAGACATCGCGACGTATTATTGTCAGCAGGGTACTAGC |
| | CTGCCTCGCGCCTTCGGCCAGGGGACCAAACTGGAAATCAAA |
| | (SEQ ID NO: 126) |
| | DIQMTQSPSSLSASVGDRVTITCQASQSISSDYLHWYQQKPGKAPKL |
| | LIYRTSNLETGVPSRFSGSGSGTDFTFTISSLQPEDIATYYCQQGTS |
| | LPRAFGQGTKLEIK |
| | (SEQ ID NO: 127) |
| 723C2VK- | GATATCCAGATGACGCAGAGCCCAAGCAGCCTGAGCGCGTCCGTGGG |
| 462-08 | CGACCGCGTGACGATCACCTGTCAGGCGTCCCAGAGCATCAGCAGCG |
| | ACTATCTGCATTGGTATCAGCAGAAACCAGGTAAAGCCCCTAAACTG |
| | CTGATCTACCGGACCTCCAATCTGGAAAGCGGCGTGCCTAGCCGTTT |
| | CAGCGGTAGCGGCTCCGGTACCGACTTCACCTTTACTATCTCCAGCC |
| | TGCAGCCTGAAGACATCGCGACGTATTATTGTCAGCAGGGTACTAGC |
| | CTGCCTCGCGCCTTCGGCCAGGGGACCAAACTGGAAATCAAA |
| | (SEQ ID NO: 128) |
| | DIQMTQSPSSLSASVGDRVTITCQASQSISSDYLHWYQQKPGKAPKL |
| | LIYRTSNLESGVPSRFSGSGSGTDFTFTISSLQPEDIATYYCQQGTS |
| | LPRAFGQGTKLEIK |
| | (SEQ ID NO: 129) |

TABLE 6
Humanized 723C2-VH Sequence

| | |
|---|---|
| 723C2VH- | GAAGTGCAGCTGGTGGAAAGCGGTGGTGGCCTGGTGCAGCCAGGCGG |
| 463-60 | CTCCCTGCGCCTGAGCTGCGCCGCAAGCGGTTTCACCTTTAGCGACT |
| | ACTATATGTCCTGGGTGCGTCAGGCGCCAGGTAAAGGTCTGGAATGG |
| | GTGTCATACATCAGCTCCGGGGGCGGTAGCAAGTACTATCCGGACCT |
| | GGTGAAAGGGCGCTTTACTATCTCCCGGGATAATGCAAAAAATAGCC |
| | TGTACCTGCAGATGAGCAGCCTGCGGGCGGAAGATACCGCCGTGTAT |
| | TACTGTGCGCGTCTGCCGCATTATTTCGCCATGGATTACTGGGGCCA |
| | GGGGACCCTGGTGACCGTGAGCAGC |
| | (SEQ ID NO: 130) |
| | EVQLVESGGGLVQPGGSLRLSCAASGFTFSDYYMSWVRQAPGKGLEW |
| | VSYISSGGGSKYYPDLVKGRFTISRDNAKNSLYLQMSSLRAEDTAVY |
| | YCARLPHYFAMDYWGQGTLVTVSS |
| | (SEQ ID NO: 131) |
| 723C2VH- | GAAGTGCAGCTGGTGGAAAGCGGTGGTGGCCTGGTGCAGCCAGGCGG |
| 461-41 | CTCCCTGCGCCTGAGCTGCGCCGCAAGCGGTTTCACCTTTAGCGACT |
| | ACTATATGTCCTGGGTGCGTCAGGCGCCAGGTAAAGGTCTGGAATGG |
| | GTGGCATACATCAGCTCCGGGGGCGGTAGCAGCTACTATCCGGACCT |
| | GGTGAAAGGGCGCTTTACTATCTCCCGGGATAATGCAAAAAATAGCC |
| | TGTACCTGCAGATGCAGAGCCTGCGGGCGGAAGATACCGCCGTGTAT |
| | TACTGTGCGCGTCTGCCGCATTATTTCGCCATGGATTACTGGGGCCA |
| | GGGGACCCTGGTGACCGTGAGCAGC |
| | (SEQ ID NO: 132) |
| | EVQLVESGGGLVQPGGSLRLSCAASGFTFSDYYMSWVRQAPGKGLEW |
| | VAYISSGGGSSYYPDLVKGRFTISRDNAKNSLYLQMQSLRAEDTAVY |
| | YCARLPHYFAMDYWGQGTLVTVSS |
| | (SEQ ID NO: 133) |
| 723C2VH- | GAAGTGCAGCTGGTGGAAAGCGGTGGTGGCCTGGTGCAGCCAGGCGG |
| 461-47 | CTCCCTGCGCCTGAGCTGCGCCGCAAGCGGTTTCACCTTTAGCGACT |
| | ACTATATGTCCTGGGTGCGTCAGGCGCCAGGTAAAGGTCTGGAATGG |
| | GTGGCATACATCAGCTCCGGGGGCGGTAGCAGCTACTATCCGGACGC |
| | TGTGAAAGGGCGCTTTACTATCTCCCGGGATAATGCAAAACAGAGCC |
| | TGTACCTGCAGATGCAGAGCCTGCGGGCGGAAGATACCGCCGTGTAT |
| | TACTGTGCGCGTCTGCCGCATTATTTCGCCATGGATTACTGGGGCCA |
| | GGGGACCCTGGTGACCGTGAGCAGC |
| | (SEQ ID NO: 134) |
| | EVQLVESGGGLVQPGGSLRLSCAASGFTFSDYYMSWVRQAPGKGLEW |
| | VAYISSGGGSSYYPDAVKGRFTISRDNAKQSLYLQMQSLRAEDTAVY |
| | YCARLPHYFAMDYWGQGTLVTVSS |
| | (SEQ ID NO: 135) |
| 723C2VH- | GAAGTGCAGCTGGTGGAAAGCGGTGGTGGCCTGGTGCAGCCAGGCGG |
| 461-44 | CTCCCTGCGCCTGAGCTGCGCCGCAAGCGGTTTCACCTTTAGCGACT |
| | ACTATATGTCCTGGGTGCGTCAGGCGCCAGGTAAAGGTCTGGAATGG |
| | GTGGCCTACATCAGCTCCGGGGGCGGTAGCAGCTACTATCCGGACCT |
| | GGTGAAAGGGCGCTTTACTATCTCCCGGGATAATGCAAAACAGAGCC |
| | TGTACCTGCAGATGAACAGCCTGCGGGCGGAAGATACCGCCGTGTAT |
| | TACTGTGCGCGTCTGCCGCATTATTTCGCCATGGATTACTGGGGCCA |
| | GGGGACCCTGGTGACCGTGAGCAGC |
| | (SEQ ID NO: 136) |
| | EVQLVESGGGLVQPGGSLRLSCAASGFTFSDYYMSWVRQAPGKGLEW |
| | VAYISSGGGSSYYPDLVKGRFTISRDNAKQSLYLQMNSLRAEDTAVY |
| | YCARLPHYFAMDYWGQGTLVTVSS |
| | (SEQ ID NO: 137) |
| 723C2VH- | GAAGTGCAGCTGGTGGAAAGCGGTGGTGGCCTGGTGCAGCCAGGCGG |
| 461-40 | CTCCCTGCGCCTGAGCTGCGCCGCAAGCGGTTTCACCTTTAGCGACT |
| | ACTATATGTCCTGGGTGCGTCAGGCGCCAGGTAAAGGTCTGGAATGG |
| | GTGGCCTACATCAGCTCCGGGGGCGGTAGCAGCTACTATCCGGACCT |
| | GGTGAAAGGGCGCTTTACTATCTCCCGGGATAATGCAAAACAGAGCC |
| | TGTACCTGCAGATGCAGAGCCTGCGGGCGGAAGATACCGCCGTGTAT |
| | TACTGTGCGCGTCTGCCGCATTATTTCGCCATGGATTACTGGGGCCA |
| | GGGGACCCTGGTGACCGTGAGCAGC |
| | (SEQ ID NO: 138) |
| | EVQLVESGGGLVQPGGSLRLSCAASGFTFSDYYMSWVRQAPGKGLEW |
| | VAYISSGGGSSYYPDLVKGRFTISRDNAKQSLYLQMQSLRAEDTAVY |
| | YCARLPHYFAMDYWGQGTLVTVSS |
| | (SEQ ID NO: 139) |

Selected combination of humanized light chain and heavy chain variable regions derived from mouse antibody 723C2 resulted in Antibodies A, B, C, D and E:
Antibody A: 723C2-IgG4Pro-463-60 with IgK-463-60 (heavy chain variable region 723C2V_{H}-463-60 and light chain variable region 723C2VK-463-60);
Antibody B: 723C2-IgG4Pro-461-41 with IgK-462-07 (heavy chain variable region 723C2V_{H}-461-41 and light chain variable region 723C2VK-462-07);
Antibody C: 723C2-IgG4Pro-461-47 with IgK-462-07 (heavy chain variable region 723C2V_{H}-461-47 and light chain variable region 723C2VK-462-07);
Antibody D: 723C2-IgG4Pro-461-44 with IgK-462-08 (heavy chain variable region 723C2V_{H}-461-44 and light chain variable region 723C2VK-462-08).
Antibody E: 723C2-IgG4Pro-461-40 with IgK-462-08 (heavy chain variable region 723C2V_{H}-461-40 and light chain variable region 723C2VK-462-08).

Antibodies A, B, C, D and E have the heavy and light chain sequences shown in Table 7.

TABLE 7
Heavy and Light Chain DNA and Amino Acid Sequences for Antibodies
A, B, C, D, and E

| | | |
|---|---|---|
| Antibody A | IgK | GATATCCAGATGACGCAGAGCCCAAGCAGCCTGAGCG |
| | light | CGTCCGTGGGCGACCGCGTGACGATCACCTGTAGCGC |
| | Chain | GTCCCAGAGCATCAGCAGCGACTATCTGCATTGGTAT |
| | #463-60 | CAGCAGAAACCAGGTAAAGCCCCTAAACTGCTGATCT |
| | | ACCGGACCTCCAATCTGGCAAGCGGCGTGCCTAGCCG |
| | | TTTCAGCGGTAGCGGCTCCGGTACCGACTTCACCTTT |
| | | ACTATCTCCAGCCTGCAGCCTGAAGACATCGCGACGT |
| | | ATTATTGTCAGCAGGGTACTAGCCTGCCTCGCGCCTT |
| | | CGGCCAGGGGACCAAACTGGAAATCAAACGTACTGTG |
| | | GCTGCACCATCTGTCTTCATCTTCCCGCCATCTGATG |
| | | AGCAATTGAAATCTGGAACTGCCTCTGTTGTGTGCCT |
| | | GCTGAATAACTTCTATCCCAGAGAGGCCAAAGTACAG |
| | | TGGAAGGTGGATAACGCCCTCCAATCGGGTAACTCCC |
| | | AGGAGAGTGTCACAGAGCAGGACAGCAAGGACAGCAC |
| | | CTACAGCCTCAGCAGCACCCTGACGCTGAGCAAAGCA |
| | | GACTACGAGAAACACAAAGTCTACGCCTGCGAAGTCA |
| | | CCCATCAGGGCCTGAGCTCGCCCGTCACAAAGAGCTT |
| | | CAACAGGGGAGAGTGT (SEQ ID NO: 140) |
| | | DIQMTQSPSSLSASVGDRVTITCSASQSISSDYLHWY |
| | | QQKPGKAPKLLIYRTSNLASGVPSRFSGSGSGTDFTF |
| | | TISSLQPEDIATYYCQQGTSLPRAFGQGTKLEIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| | | (SEQ ID NO: 141) |
| | IgG4Pro | GAAGTGCAGCTGGTGGAAAGCGGTGGTGGCCTGGTGC |
| | Heavy | AGCCAGGCGGCTCCCTGCGCCTGAGCTGCGCCGCAAG |
| | Chain | CGGTTTCACCTTTAGCGACTACTATATGTCCTGGGTG |
| | #463-60 | CGTCAGGCGCCAGGTAAAGGTCTGGAATGGGTGTCAT |
| | | ACATCAGCTCCGGGGGCGGTAGCAAGTACTATCCGGA |
| | | CCTGGTGAAAGGGCGCTTTACTATCTCCCGGGATAAT |
| | | GCAAAAAATAGCCTGTACCTGCAGATGAGCAGCCTGC |
| | | GGGCGGAAGATACCGCCGTGTATTACTGTGCGCGTCT |
| | | GCCGCATTATTTCGCCATGGATTACTGGGGCCAGGGG |
| | | ACCCTGGTGACCGTGAGCAGCGCCTCCACAAAGGGCC |
| | | CTTCCGTGTTCCCCCTGGCCCCTTGCTCCCGGTCCAC |
| | | CTCCGAGTCTACCGCCGCTCTGGGCTGCCTGGTCAAG |
| | | GACTACTTCCCCGAGCCCGTGACCGTGTCCTGGAACT |
| | | CTGGCGCCCTGACCTCCGGCGTGCACACCTTCCCTGC |
| | | TGTGCTGCAGTCCTCCGGCCTGTACTCCCTGTCCTCC |
| | | GTCGTGACCGTGCCCTCCTCTAGCCTGGGCACCAAGA |
| | | CCTACACCTGTAACGTGGACCACAAGCCCTCCAACAC |
| | | CAAGGTGGACAAGCGGGTGGAATCTAAGTACGGCCCT |
| | | CCCTGCCCCCCCTGCCCTGCCCCTGAATTTCTGGGCG |
| | | GACCCTCCGTGTTCCTGTTCCCCCCAAAGCCCAAGGA |
| | | CACCCTGATGATCTCCCGGACCCCCGAAGTGACCTGC |
| | | GTGGTGGTGGACGTGTCCCAGGAAGATCCCGAGGTCC |
| | | AGTTTAATTGGTACGTGGACGGCGTGGAAGTGCACAA |
| | | CGCCAAGACCAAGCCCAGAGAGGAACAGTTCAACTCC |
| | | ACCTACCGGGTGGTGTCCGTGCTGACCGTGCTGCACC |
| | | AGGACTGGCTGAACGGCAAAGAGTACAAGTGCAAGGT |
| | | GTCCAACAAGGGCCTGCCCTCCAGCATCGAAAAGACC |
| | | ATCTCCAAGGCCAAGGGCCAGCCCCGCGAGCCCCAGG |
| | | TGTACACCCTGCCTCCAAGCCAGGAAGAGATGACCAA |
| | | GAACCAGGTGTCCCTGACCTGTCTGGTCAAGGGCTTC |
| | | TACCCCTCCGATATCGCCGTGGAATGGGAGTCCAACG |
| | | GCCAGCCCGAGAACAACTACAAGACCACCCCCCCTGT |
| | | GCTGGACTCCGACGGCTCCTTCTTCCTGTACTCTCGG |
| | | CTGACCGTGGACAAGTCCCGGTGGCAGGAAGGCAACG |
| | | TCTTCTCCTGCTCCGTGATGCACGAGGCCCTGCACAA |
| | | CCACTACACCCAGAAGTCCCTGTCCCTGAGCCTGGGC |
| | | (SEQ ID NO: 142) |
| | | EVQLVESGGGLVQPGGSLRLSCAASGFTFSDYYMSWV |
| | | RQAPGKGLEWVSYISSGGGSKYYPDLVKGRFTISRDN |
| | | AKNSLYLQMSSLRAEDTAVYYCARLPHYFAMDYWGQG |
| | | TLVTVSSASTKGPSVFPLAPCSRSTSESTAALGCLVK |
| | | DYFPEPVTVSWNSGALTSGVHTFPAVLQSSGLYSLSS |
| | | VVTVPSSSLGTKTYTCNVDHKPSNTKVDKRVESKYGP |
| | | 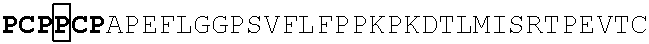 |
| | | VVVDVSQEDPEVQFNWYVDGVEVHNAKTKPREEQFNS |
| | | TYRVVSVLTVLHQDWLNGKEYKCKVSNKGLPSSIEKT |
| | | ISKAKGQPREPQVYTLPPSQEEMTKNQVSLTCLVKGF |
| | | YPSDIAVEWESNGQPENNYKTTPPVLDSDGSFFLYSR |
| | | LTVDKSRWQEGNVFSCSVMHEALHNHYTQKSLSLSLG |
| | | (SEQ ID NO: 143) |
| Antibody B | IgK | GACATCCAGATGACCCAGAGCCCAAGCAGCCTGAGCG |
| | light | CCAGCGTGGGCGACCGCGTGACCATCACCTGCCAGGC |
| | Chain | CAGCCAGAGCATCAGCAGCGACTACCTGCACTGGTAC |
| | #462-07 | CAGCAGAAGCCAGGCAAGGCCCCAAAGCTGCTGATCT |
| | | ACCGCACCAGCAACCTGGAGACCGGCGTGCCAAGCCG |
| | | CTTCAGCGGCAGCGGCAGCGGCACCGACTTCACCTTC |
| | | ACCATCAGCAGCCTGCAGCCAGAGGACATCGCCACCT |
| | | ACTACTGCCAGCAGGGCACCAGCCTGCCACGCGCCTT |
| | | CGGCCAGGGCACCAAGCTGGAGATCAAGCGTACTGTG |
| | | GCTGCACCATCTGTCTTCATCTTCCCGCCATCTGATG |
| | | AGCAATTGAAATCTGGAACTGCCTCTGTTGTGTGCCT |
| | | GCTGAATAACTTCTATCCCAGAGAGGCCAAAGTACAG |
| | | TGGAAGGTGGATAACGCCCTCCAATCGGGTAACTCCC |
| | | AGGAGAGTGTCACAGAGCAGGACAGCAAGGACAGCAC |
| | | CTACAGCCTCAGCAGCACCCTGACGCTGAGCAAAGCA |
| | | GACTACGAGAAACACAAAGTCTACGCCTGCGAAGTCA |
| | | CCCATCAGGGCCTGAGCTCGCCCGTCACAAAGAGCTT |
| | | CAACAGGGGAGAGTGT (SEQ ID NO: 144) |
| | | DIQMTQSPSSLSASVGDRVTITCQASQSISSDYLHWY |
| | | QQKPGKAPKLLIYRTSNLETGVPSRFSGSGSGTDFTF |
| | | TISSLQPEDIATYYCQQGTSLPRAFGQGTKLEIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| | | (SEQ ID NO: 145) |
| | IgG4Pro | GAAGTGCAGCTGGTGGAAAGCGGCGGAGGCCTGGTGC |
| | Heavy | AGCCAGGCGGCAGCCTGAGACTGAGCTGCGCCGCCAG |
| | Chain | CGGCTTCACCTTCAGCGACTACTACATGAGCTGGGTG |
| | #461-41 | CGCCAGGCCCCAGGCAAGGGCCTGGAGTGGGTGGCCT |
| | | ACATCAGCAGCGGCGGCGGCAGCAGCTACTACCCAGA |
| | | CCTGGTGAAGGGCCGCTTCACCATCAGCCGCGACAAC |
| | | GCCAAGAACAGCCTGTACCTGCAGATGCAGAGCCTGC |
| | | GCGCCGAGGACACCGCCGTGTACTACTGCGCCCGCCT |
| | | GCCACACTACTTCGCCATGGACTACTGGGGCCAGGGC |
| | | ACCCTGGTGACCGTGAGCAGCGCCTCCACAAAGGGCC |
| | | CTTCCGTGTTCCCCCTGGCCCCTTGCTCCCGGTCCAC |
| | | CTCCGAGTCTACCGCCGCTCTGGGCTGCCTGGTCAAG |
| | | GACTACTTCCCCGAGCCCGTGACCGTGTCCTGGAACT |
| | | CTGGCGCCCTGACCTCCGGCGTGCACACCTTCCCTGC |
| | | TGTGCTGCAGTCCTCCGGCCTGTACTCCCTGTCCTCC |
| | | GTCGTGACCGTGCCCTCCTCTAGCCTGGGCACCAAGA |
| | | CCTACACCTGTAACGTGGACCACAAGCCCTCCAACAC |
| | | CAAGGTGGACAAGCGGGTGGAATCTAAGTACGGCCCT |
| | | CCCTGCCCCCCCTGCCCTGCCCCTGAATTTCTGGGCG |
| | | GACCCTCCGTGTTCCTGTTCCCCCCAAAGCCCAAGGA |
| | | CACCCTGATGATCTCCCGGACCCCCGAAGTGACCTGC |
| | | GTGGTGGTGGACGTGTCCCAGGAAGATCCCGAGGTCC |
| | | AGTTTAATTGGTACGTGGACGGCGTGGAAGTGCACAA |
| | | CGCCAAGACCAAGCCCAGAGAGGAACAGTTCAACTCC |
| | | ACCTACCGGGTGGTGTCCGTGCTGACCGTGCTGCACC |
| | | AGGACTGGCTGAACGGCAAAGAGTACAAGTGCAAGGT |
| | | GTCCAACAAGGGCCTGCCCTCCAGCATCGAAAAGACC |
| | | ATCTCCAAGGCCAAGGGCCAGCCCCGCGAGCCCCAGG |
| | | TGTACACCCTGCCTCCAAGCCAGGAAGAGATGACCAA |
| | | GAACCAGGTGTCCCTGACCTGTCTGGTCAAGGGCTTC |
| | | TACCCCTCCGATATCGCCGTGGAATGGGAGTCCAACG |
| | | GCCAGCCCGAGAACAACTACAAGACCACCCCCCCTGT |
| | | GCTGGACTCCGACGGCTCCTTCTTCCTGTACTCTCGG |
| | | CTGACCGTGGACAAGTCCCGGTGGCAGGAAGGCAACG |
| | | TCTTCTCCTGCTCCGTGATGCACGAGGCCCTGCACAA |
| | | CCACTACACCCAGAAGTCCCTGTCCCTGAGCCTGGGC |
| | | (SEQ ID NO: 146) |
| | | EVQLVESGGGLVQPGGSLRLSCAASGFTFSDYYMSWV |
| | | RQAPGKGLEWVAYISSGGGSSYYPDLVKGRFTISRDN |
| | | AKNSLYLQMQSLRAEDTAVYYCARLPHYFAMDYWGQG |
| | | TLVTVSSASTKGPSVFPLAPCSRSTSESTAALGCLVK |
| | | DYFPEPVTVSWNSGALTSGVHTFPAVLQSSGLYSLSS |
| | | VVTVPSSSLGTKTYTCNVDHKPSNTKVDKRVESKYGP |
| | | 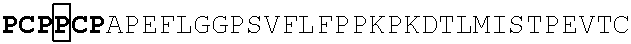 |
| | | VVVDVSQEDPEVQFNWYVDGVEVHNAKTKPREEQFNS |
| | | TYRVVSVLTVLHQDWLNGKEYKCKVSNKGLPSSIEKT |
| | | ISKAKGQPREPQVYTLPPSQEEMTKNQVSLTCLVKGF |
| | | YPSDIAVEWESNGQPENNYKTTPPVLDSDGSFFLYSR |
| | | LTVDKSRWQEGNVFSCSVMHEALHNHYTQKSLSLSLG |
| | | (SEQ ID NO: 147) |
| Antibody C | IgK | GACATCCAGATGACCCAGAGCCCAAGCAGCCTGAGCG |
| | light | CCAGCGTGGGCGACCGCGTGACCATCACCTGCCAGGC |
| | Chain | CAGCCAGAGCATCAGCAGCGACTACCTGCACTGGTAC |
| | #462-07 | CAGCAGAAGCCAGGCAAGGCCCCAAAGCTGCTGATCT |
| | | ACCGCACCAGCAACCTGGAGACCGGCGTGCCAAGCCG |
| | | CTTCAGCGGCAGCGGCAGCGGCACCGACTTCACCTTC |
| | | ACCATCAGCAGCCTGCAGCCAGAGGACATCGCCACCT |
| | | ACTACTGCCAGCAGGGCACCAGCCTGCCACGCGCCTT |
| | | CGGCCAGGGCACCAAGCTGGAGATCAAGCGTACTGTG |
| | | GCTGCACCATCTGTCTTCATCTTCCCGCCATCTGATG |
| | | AGCAATTGAAATCTGGAACTGCCTCTGTTGTGTGCCT |
| | | GCTGAATAACTTCTATCCCAGAGAGGCCAAAGTACAG |
| | | TGGAAGGTGGATAACGCCCTCCAATCGGGTAACTCCC |
| | | AGGAGAGTGTCACAGAGCAGGACAGCAAGGACAGCAC |
| | | CTACAGCCTCAGCAGCACCCTGACGCTGAGCAAAGCA |
| | | GACTACGAGAAACACAAAGTCTACGCCTGCGAAGTCA |
| | | CCCATCAGGGCCTGAGCTCGCCCGTCACAAAGAGCTT |
| | | CAACAGGGGAGAGTGT (SEQ ID NO: 144) |
| | | DIQMTQSPSSLSASVGDRVTITCQASQSISSDYLHWY |
| | | QQKPGKAPKLLIYRTSNLETGVPSRFSGSGSGTDFTF |
| | | TISSLQPEDIATYYCQQGTSLPRAFGQGTKLEIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| | | (SEQ ID NO: 145) |
| | IgG4Pro | GAGGTGCAGCTGGTGGAGAGCGGCGGCGGCCTGGTGC |
| | Heavy | AGCCAGGTGGTAGCCTGCGCCTGAGCTGCGCCGCCAG |
| | Chain | CGGCTTCACCTTCAGCGACTACTACATGAGCTGGGTG |
| | #461-47 | CGCCAGGCTCCAGGCAAGGGTCTGGAATGGGTGGCCT |
| | | ACATCAGCAGCGGCGGCGGCAGCAGCTACTACCCAGA |
| | | CGCCGTGAAGGGCCGCTTCACCATCAGCCGCGACAAC |
| | | GCCAAGCAGAGCCTGTACCTGCAGATGCAGAGCCTGC |
| | | GCGCCGAGGACACCGCCGTGTACTACTGCGCCCGCCT |
| | | GCCACACTACTTCGCCATGGACTACTGGGGCCAGGGC |
| | | ACCCTGGTGACCGTGAGCAGCGCCTCCACAAAGGGCC |
| | | CTTCCGTGTTCCCCCTGGCCCCTTGCTCCCGGTCCAC |
| | | CTCCGAGTCTACCGCCGCTCTGGGCTGCCTGGTCAAG |
| | | GACTACTTCCCCGAGCCCGTGACCGTGTCCTGGAACT |
| | | CTGGCGCCCTGACCTCCGGCGTGCACACCTTCCCTGC |
| | | TGTGCTGCAGTCCTCCGGCCTGTACTCCCTGTCCTCC |
| | | GTCGTGACCGTGCCCTCCTCTAGCCTGGGCACCAAGA |
| | | CCTACACCTGTAACGTGGACCACAAGCCCTCCAACAC |
| | | CAAGGTGGACAAGCGGGTGGAATCTAAGTACGGCCCT |
| | | CCCTGCCCCCCCTGCCCTGCCCCTGAATTTCTGGGCG |
| | | GACCCTCCGTGTTCCTGTTCCCCCCAAAGCCCAAGGA |
| | | CACCCTGATGATCTCCCGGACCCCCGAAGTGACCTGC |
| | | GTGGTGGTGGACGTGTCCCAGGAAGATCCCGAGGTCC |
| | | AGTTTAATTGGTACGTGGACGGCGTGGAAGTGCACAA |
| | | CGCCAAGACCAAGCCCAGAGAGGAACAGTTCAACTCC |
| | | ACCTACCGGGTGGTGTCCGTGCTGACCGTGCTGCACC |
| | | AGGACTGGCTGAACGGCAAAGAGTACAAGTGCAAGGT |
| | | GTCCAACAAGGGCCTGCCCTCCAGCATCGAAAAGACC |
| | | ATCTCCAAGGCCAAGGGCCAGCCCCGCGAGCCCCAGG |
| | | TGTACACCCTGCCTCCAAGCCAGGAAGAGATGACCAA |
| | | GAACCAGGTGTCCCTGACCTGTCTGGTCAAGGGCTTC |
| | | TACCCCTCCGATATCGCCGTGGAATGGGAGTCCAACG |
| | | GCCAGCCCGAGAACAACTACAAGACCACCCCCCCTGT |
| | | GCTGGACTCCGACGGCTCCTTCTTCCTGTACTCTCGG |
| | | CTGACCGTGGACAAGTCCCGGTGGCAGGAAGGCAACG |
| | | TCTTCTCCTGCTCCGTGATGCACGAGGCCCTGCACAA |
| | | CCACTACACCCAGAAGTCCCTGTCCCTGAGCCTGGGC |
| | | (SEQ ID NO: 148) |
| | | EVQLVESGGGLVQPGGSLRLSCAASGFTFSDYYMSWV |
| | | RQAPGKGLEWVAYISSGGGSSYYPDAVKGRFTISRDN |
| | | AKQSLYLQMQSLRAEDTAVYYCARLPHYFAMDYWGQG |
| | | TLVTVSSASTKGPSVFPLAPCSRSTSESTAALGCLVK |
| | | DYFPEPVTVSWNSGALTSGVHTFPAVLQSSGLYSLSS |
| | | VVTVPSSSLGTKTYTCNVDHKPSNTKVDKRVESKYGP |
| | | 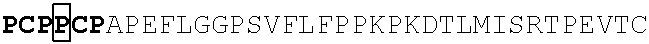 |
| | | VVVDVSQEDPEVQFNWYVDGVEVHNAKTKPREEQFNS |
| | | TYRVVSVLTVLHQDWLNGKEYKCKVSNKGLPSSIEKT |
| | | ISKAKGQPREPQVYTLPPSQEEMTKNQVSLTCLVKGF |
| | | YPSDIAVEWESNGQPENNYKTTPPVLDSDGSFFLYSR |
| | | LTVDKSRWQEGNVFSCSVMHEALHNHYTQKSLSLSLG |
| | | (SEQ ID NO: 149) |
| Antibody D | IgK | GACATCCAGATGACCCAGAGCCCAAGCAGCCTGAGCG |
| | light | CCAGCGTGGGCGACCGCGTGACCATCACCTGCCAGGC |
| | Chain | CAGCCAGAGCATCAGCAGCGACTACCTGCACTGGTAC |
| | #462-08 | CAGCAGAAGCCAGGCAAGGCCCCAAAGCTGCTGATCT |
| | | ACCGCACCAGCAACCTGGAGAGCGGCGTGCCAAGCCG |
| | | CTTCAGCGGCAGCGGCAGCGGCACCGACTTCACCTTC |
| | | ACCATCAGCAGCCTGCAGCCAGAGGACATCGCCACCT |
| | | ACTACTGCCAGCAGGGCACCAGCCTGCCACGCGCCTT |
| | | CGGCCAGGGCACCAAGCTGGAGATCAAGCGTACTGTG |
| | | GCTGCACCATCTGTCTTCATCTTCCCGCCATCTGATG |
| | | AGCAATTGAAATCTGGAACTGCCTCTGTTGTGTGCCT |
| | | GCTGAATAACTTCTATCCCAGAGAGGCCAAAGTACAG |
| | | TGGAAGGTGGATAACGCCCTCCAATCGGGTAACTCCC |
| | | AGGAGAGTGTCACAGAGCAGGACAGCAAGGACAGCAC |
| | | CTACAGCCTCAGCAGCACCCTGACGCTGAGCAAAGCA |
| | | GACTACGAGAAACACAAAGTCTACGCCTGCGAAGTCA |
| | | CCCATCAGGGCCTGAGCTCGCCCGTCACAAAGAGCTT |
| | | CAACAGGGGAGAGTGT (SEQ ID NO: 150) |
| | | DIQMTQSPSSLSASVGDRVTITCQASQSISSDYLHWY |
| | | QQKPGKAPKLLIYRTSNLESGVPSRFSGSGSGTDFTF |
| | | TISSLQPEDIATYYCQQGTSLPRAFGQGTKLEIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| | | (SEQ ID NO: 151) |
| | IgG4Pro | GAAGTGCAGCTGGTGGAAAGCGGTGGTGGCCTGGTGC |
| | Heavy | AGCCAGGCGGCTCCCTGCGCCTGAGCTGCGCCGCAAG |
| | Chain | CGGTTTCACCTTTAGCGACTACTATATGTCCTGGGTG |
| | #461-44 | CGTCAGGCGCCAGGTAAAGGTCTGGAATGGGTGGCCT |
| | | ACATCAGCTCCGGGGGCGGTAGCAGCTACTATCCGGA |
| | | CCTGGTGAAAGGGCGCTTTACTATCTCCCGGGATAAT |
| | | GCAAAACAGAGCCTGTACCTGCAGATGAACAGCCTGC |
| | | GGGCGGAAGATACCGCCGTGTATTACTGTGCGCGTCT |
| | | GCCGCATTATTTCGCCATGGATTACTGGGGCCAGGGG |
| | | ACCCTGGTGACCGTGAGCAGCGCCTCCACAAAGGGCC |
| | | CTTCCGTGTTCCCCCTGGCCCCTTGCTCCCGGTCCAC |
| | | CTCCGAGTCTACCGCCGCTCTGGGCTGCCTGGTCAAG |
| | | GACTACTTCCCCGAGCCCGTGACCGTGTCCTGGAACT |
| | | CTGGCGCCCTGACCTCCGGCGTGCACACCTTCCCTGC |
| | | TGTGCTGCAGTCCTCCGGCCTGTACTCCCTGTCCTCC |
| | | GTCGTGACCGTGCCCTCCTCTAGCCTGGGCACCAAGA |
| | | CCTACACCTGTAACGTGGACCACAAGCCCTCCAACAC |
| | | CAAGGTGGACAAGCGGGTGGAATCTAAGTACGGCCCT |
| | | CCCTGCCCCCCCTGCCCTGCCCCTGAATTTCTGGGCG |
| | | GACCCTCCGTGTTCCTGTTCCCCCCAAAGCCCAAGGA |
| | | CACCCTGATGATCTCCCGGACCCCCGAAGTGACCTGC |
| | | GTGGTGGTGGACGTGTCCCAGGAAGATCCCGAGGTCC |
| | | AGTTTAATTGGTACGTGGACGGCGTGGAAGTGCACAA |
| | | CGCCAAGACCAAGCCCAGAGAGGAACAGTTCAACTCC |
| | | ACCTACCGGGTGGTGTCCGTGCTGACCGTGCTGCACC |
| | | AGGACTGGCTGAACGGCAAAGAGTACAAGTGCAAGGT |
| | | GTCCAACAAGGGCCTGCCCTCCAGCATCGAAAAGACC |
| | | ATCTCCAAGGCCAAGGGCCAGCCCCGCGAGCCCCAGG |
| | | TGTACACCCTGCCTCCAAGCCAGGAAGAGATGACCAA |
| | | GAACCAGGTGTCCCTGACCTGTCTGGTCAAGGGCTTC |
| | | TACCCCTCCGATATCGCCGTGGAATGGGAGTCCAACG |
| | | GCCAGCCCGAGAACAACTACAAGACCACCCCCCCTGT |
| | | GCTGGACTCCGACGGCTCCTTCTTCCTGTACTCTCGG |
| | | CTGACCGTGGACAAGTCCCGGTGGCAGGAAGGCAACG |
| | | TCTTCTCCTGCTCCGTGATGCACGAGGCCCTGCACAA |
| | | CCACTACACCCAGAAGTCCCTGTCCCTGAGCCTGGGC |
| | | (SEQ ID NO: 152) |
| | | EVQLVESGGGLVQPGGSLRLSCAASGFTFSDYYMSWV |
| | | RQAPGKGLEWVAYISSGGGSSYYPDLVKGRFTISRDN |
| | | AKQSLYLQMNSLRAEDTAVYYCARLPHYFAMDYWGQG |
| | | TLVTVSSASTKGPSVFPLAPCSRSTSESTAALGCLVK |
| | | DYFPEPVTVSWNSGALTSGVHTFPAVLQSSGLYSLSS |
| | | VVIVPSSSLGIKTYTCNVDHKPSNIKVDKRVESKYGP |
| | | 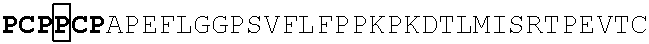 |
| | | VVVDVSQEDPEVQFNWYVDGVEVHNAKTKPREEQFNS |
| | | TYRVVSVLTVLHQDWLNGKEYKCKVSNKGLPSSIEKT |
| | | ISKAKGQPREPQVYTLPPSQEEMTKNQVSLTCLVKGF |
| | | YPSDIAVEWESNGQPENNYKTTPPVLDSDGSFFLYSR |
| | | LTVDKSRWQEGNVFSCSVMHEALHNHYTQKSLSLSLG |
| | | (SEQ ID NO: 153) |
| Antibody E | IgK | GACATCCAGATGACCCAGAGCCCAAGCAGCCTGAGCG |
| | light | CCAGCGTGGGCGACCGCGTGACCATCACCTGCCAGGC |
| | Chain | CAGCCAGAGCATCAGCAGCGACTACCTGCACTGGTAC |
| | #462-08 | CAGCAGAAGCCAGGCAAGGCCCCAAAGCTGCTGATCT |
| | | ACCGCACCAGCAACCTGGAGAGCGGCGTGCCAAGCCG |
| | | CTTCAGCGGCAGCGGCAGCGGCACCGACTTCACCTTC |
| | | ACCATCAGCAGCCTGCAGCCAGAGGACATCGCCACCT |
| | | ACTACTGCCAGCAGGGCACCAGCCTGCCACGCGCCTT |
| | | CGGCCAGGGCACCAAGCTGGAGATCAAGCGTACTGTG |
| | | GCTGCACCATCTGTCTTCATCTTCCCGCCATCTGATG |
| | | AGCAATTGAAATCTGGAACTGCCTCTGTTGTGTGCCT |
| | | GCTGAATAACTTCTATCCCAGAGAGGCCAAAGTACAG |
| | | TGGAAGGTGGATAACGCCCTCCAATCGGGTAACTCCC |
| | | AGGAGAGTGTCACAGAGCAGGACAGCAAGGACAGCAC |
| | | CTACAGCCTCAGCAGCACCCTGACGCTGAGCAAAGCA |
| | | GACTACGAGAAACACAAAGTCTACGCCTGCGAAGTCA |
| | | CCCATCAGGGCCTGAGCTCGCCCGTCACAAAGAGCTT |
| | | CAACAGGGGAGAGTGT (SEQ ID NO:150) |
| | | DIQMTQSPSSLSASVGDRVTITCQASQSISSDYLHWY |
| | | QQKPGKAPKLLIYRTSNLESGVPSRFSGSGSGTDFTF |
| | | TISSLQPEDIATYYCQQGTSLPRAFGQGTKLEIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| | | (SEQ ID NO: 151) |
| | IgG4Pro | GAGGTGCAGCTGGTGGAGAGCGGCGGCGGCCTGGTGC |
| | Heavy | AGCCAGGTGGTAGCCTGCGCCTGAGCTGCGCCGCCAG |
| | Chain | CGGCTTCACCTTCAGCGACTACTACATGAGCTGGGTG |
| | #461-40 | CGCCAGGCTCCAGGCAAGGGTCTGGAATGGGTGGCCT |
| | | ACATCAGCAGCGGCGGCGGCAGCAGCTACTACCCAGA |
| | | CCTGGTGAAGGGCCGCTTCACCATCAGCCGCGACAAC |
| | | GCCAAGCAGAGCCTGTACCTGCAGATGCAGAGCCTGC |
| | | GCGCCGAGGACACCGCCGTGTACTACTGCGCCCGCCT |
| | | GCCACACTACTTCGCCATGGACTACTGGGGCCAGGGC |
| | | ACCCTGGTGACCGTGAGCAGCGCCTCCACAAAGGGCC |
| | | CTTCCGTGTTCCCCCTGGCCCCTTGCTCCCGGTCCAC |
| | | CTCCGAGTCTACCGCCGCTCTGGGCTGCCTGGTCAAG |
| | | GACTACTTCCCCGAGCCCGTGACCGTGTCCTGGAACT |
| | | CTGGCGCCCTGACCTCCGGCGTGCACACCTTCCCTGC |
| | | TGTGCTGCAGTCCTCCGGCCTGTACTCCCTGTCCTCC |
| | | GTCGTGACCGTGCCCTCCTCTAGCCTGGGCACCAAGA |
| | | CCTACACCTGTAACGTGGACCACAAGCCCTCCAACAC |
| | | CAAGGTGGACAAGCGGGTGGAATCTAAGTACGGCCCT |
| | | CCCTGCCCCCCCTGCCCTGCCCCTGAATTTCTGGGCG |
| | | GACCCTCCGTGTTCCTGTTCCCCCCAAAGCCCAAGGA |
| | | CACCCTGATGATCTCCCGGACCCCCGAAGTGACCTGC |
| | | GTGGTGGTGGACGTGTCCCAGGAAGATCCCGAGGTCC |
| | | AGTTTAATTGGTACGTGGACGGCGTGGAAGTGCACAA |
| | | CGCCAAGACCAAGCCCAGAGAGGAACAGTTCAACTCC |
| | | ACCTACCGGGTGGTGTCCGTGCTGACCGTGCTGCACC |
| | | AGGACTGGCTGAACGGCAAAGAGTACAAGTGCAAGGT |
| | | GTCCAACAAGGGCCTGCCCTCCAGCATCGAAAAGACC |
| | | ATCTCCAAGGCCAAGGGCCAGCCCCGCGAGCCCCAGG |
| | | TGTACACCCTGCCTCCAAGCCAGGAAGAGATGACCAA |
| | | GAACCAGGTGTCCCTGACCTGTCTGGTCAAGGGCTTC |
| | | TACCCCTCCGATATCGCCGTGGAATGGGAGTCCAACG |
| | | GCCAGCCCGAGAACAACTACAAGACCACCCCCCCTGT |
| | | GCTGGACTCCGACGGCTCCTTCTTCCTGTACTCTCGG |
| | | CTGACCGTGGACAAGTCCCGGTGGCAGGAAGGCAACG |
| | | TCTTCTCCTGCTCCGTGATGCACGAGGCCCTGCACAA |
| | | CCACTACACCCAGAAGTCCCTGTCCCTGAGCCTGGGC |
| | | (SEQ ID NO: 154) |
| | | EVQLVESGGGLVQPGGSLRLSCAASGFTFSDYYMSWV |
| | | RQAPGKGLEWVAYISSGGGSSYYPDLVKGRFTISRDN |
| | | AKQSLYLQMQSLRAEDTAVYYCARLPHYFAMDYWGQG |
| | | TLVTVSSASTKGPSVFPLAPCSRSTSESTAALGCLVK |
| | | DYFPEPVTVSWNSGALTSGVHTFPAVLQSSGLYSLSS |
| | | VVTVPSSSLGTKTYTCNVDHKPSNTKVDKRVESKYGP |
| | | 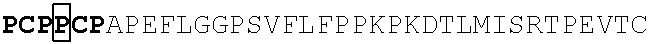 |
| | | VVVDVSQEDPEVQFNWYVDGVEVHNAKTKPREEQFNS |
| | | TYRVVSVLTVLHQDWLNGKEYKCKVSNKGLPSSIEKT |
| | | ISKAKGQPREPQVYTLPPSQEEMTKNQVSLTCLVKGF |
| | | YPSDIAVEWESNGQPENNYKTTPPVLDSDGSFFLYSR |
| | | LTVDKSRWQEGNVFSCSVMHEALHNHYTQKSLSLSLG |
| | | (SEQ ID NO: 155) |

Light chains and heavy chain variable regions of Antibodies A, B, C, D, and E are underlined in Table 7. The hinge region in the heavy chain constant regions is shown in bold with the Ser228Pro mutation boxed.

Mouse lead 723C2 was also converted to human IgG1WT, IgG1KO, and IgG4Pro formats. IgG4Pro has one mutation in the hinge region, Ser228Pro, which prevents Fab-arm exchange. IgG1KO has two mutations in the hinge region, Leu234Ala and Leu235Ala, to reduce effector function (ADCC).

Chimeric 723C2 in human IgG1WT, IgG1KO, and IgG4Pro formats are shown in Table 8.

TABLE 8
Heavy and Light Chain DNA and Amino Acid Sequences
for chimeric 723C2 in human IgG1WT, IgG1KO and IgG4Pro

| | | |
|---|---|---|
| Chimeric 723C2 | IgK light | GAGATCGTGCTGACACAGAGCCCTACCACAATGGCCG |
| in IgG1WT | Chain | CCTCTCCAGGCGAGAAGATCACCATCACATGTAGCGC |
| (723-IgG1WT) | | CAGCAGCAGCATCAGCAGCGACTACCTGCACTGGTAT |
| | | CAGCAGAAGCCTGGCTTCAGCCCCGAGCTGCTGATCT |
| | | ACAGAACAAGCAATCTGGCCAGCGGCGTGCCAGCCAG |
| | | ATTTTCTGGTTCTGGCAGCGGCACCAGCTACAGCCTG |
| | | ACAATCGGATCCATGGAAGCCGAGGACGTGGCCACCT |
| | | ATTACTGTCAGCAGGGCACAAGCCTGCCTAGAGCCTT |
| | | TGGCGGAGGCACCAAGCTGGAAATCAAGCGTACTGTG |
| | | GCTGCACCATCTGTCTTCATCTTCCCGCCATCTGATG |
| | | AGCAATTGAAATCTGGAACTGCCTCTGTTGTGTGCCT |
| | | GCTGAATAACTTCTATCCCAGAGAGGCCAAAGTACAG |
| | | TGGAAGGTGGATAACGCCCTCCAATCGGGTAACTCCC |
| | | AGGAGAGTGTCACAGAGCAGGACAGCAAGGACAGCAC |
| | | CTACAGCCTCAGCAGCACCCTGACGCTGAGCAAAGCA |
| | | GACTACGAGAAACACAAAGTCTACGCCTGCGAAGTCA |
| | | CCCATCAGGGCCTGAGCTCGCCCGTCACAAAGAGCTT |
| | | CAACAGGGGAGAGTGT |
| | | (SEQ ID NO: 156) |
| | | EIVLTQSPTTMAASPGEKITITCSASSSISSDYLHWY |
| | | QQKPGFSPELLIYRTSNLASGVPARFSGSGSGTSYSL |
| | | TIGSMEAEDVATYYCQQGTSLPRAFGGGTKLEIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| | | (SEQ ID NO: 157) |
| | IgG1WT heavy | GAAGTGCAGCTGGTGGAATCTGGCGGAGGACTTGTTC |
| | Chain | AACCTGGCGGCAGCCTGAAACTGTCTTGTGCCGCCAG |
| | | CGGCTTCACCTTCAGCGACTACTACATGAGCTGGGTC |
| | | CGACAGACCCCTGAGAAGAGACTGGAATGGGTCGCCT |
| | | ACATCAGCTCTGGCGGCGGAAGCAGCTACTACCCTGA |
| | | TAGCGTGAAGGGCAGATTCACCATCAGCCGGGACAAC |
| | | ACCAAGAACACCCTGTACCTGCAGATGTCCAGCCTGA |
| | | AGTCTGAGGACACCGCCGTGTACTACTGTGCCAGACT |
| | | GCCTCACTACTTCGCCATGGATTATTGGGGCCAGGGC |
| | | ACCAGCGTGACCGTTTCTTCTGCCTCCACCAAGGGCC |
| | | CATCGGTCTTCCCGCTAGCACCCTCCTCCAAGAGCAC |
| | | CTCTGGGGGCACAGCGGCCCTGGGCTGCCTGGTCAAG |
| | | GACTACTTCCCCGAACCGGTGACGGTGTCGTGGAACT |
| | | CAGGCGCCCTGACCAGCGGCGTGCACACCTTCCCGGC |
| | | TGTCCTACAGTCCTCAGGACTCTACTCCCTCAGCAGC |
| | | GTGGTGACCGTGCCCTCCAGCAGCTTGGGCACCCAGA |
| | | CCTACATCTGCAACGTGAATCACAAGCCCAGCAACAC |
| | | CAAGGTGGACAAGCGCGTTGAGCCCAAATCTTGTGAC |
| | | AAAACTCACACATGCCCACCGTGCCCAGCACCTGAAC |
| | | TACTAGGGGGACCGTCAGTCTTCCTCTTCCCCCCAAA |
| | | ACCCAAGGACACCCTCATGATCTCCCGGACCCCTGAG |
| | | GTCACATGCGTGGTGGTGGACGTGAGCCACGAAGACC |
| | | CTGAGGTCAAGTTCAACTGGTACGTGGACGGCGTGGA |
| | | GGTGCATAATGCCAAGACAAAGCCGCGGGAGGAGCAG |
| | | TACAACAGCACGTACCGTGTGGTCAGCGTCCTCACCG |
| | | TCCTGCACCAGGACTGGCTGAATGGCAAGGAGTACAA |
| | | GTGCAAGGTCTCCAACAAAGCCCTCCCAGCCCCCATC |
| | | GAGAAAACCATCTCCAAAGCCAAAGGGCAGCCCCGAG |
| | | AACCACAGGTGTACACCCTGCCCCCATCCCGCGAGGA |
| | | GATGACCAAGAACCAGGTAAGTTTGACCTGCCTGGTC |
| | | AAAGGCTTCTATCCCAGCGACATCGCCGTGGAGTGGG |
| | | AGAGCAATGGGCAGCCGGAGAACAACTACAAGACCAC |
| | | GCCTCCCGTGCTGGACTCCGACGGCTCCTTCTTCCTC |
| | | TATAGCAAGCTCACCGTGGACAAGAGCAGGTGGCAGC |
| | | AGGGGAACGTCTTCTCATGCTCCGTGATGCATGAGGC |
| | | TCTGCACAACCACTACACGCAGAAGAGCCTCTCCCTG |
| | | TCTCCGGGT |
| | | (SEQ ID NO: 158) |
| | | EVQLVESGGGLVQPGGSLKLSCAASGFTFSDYYMSWV |
| | | RQTPEKRLEWVAYISSGGGSSYYPDSVKGRFTISRDN |
| | | TKNTLYLQMSSLKSEDTAVYYCARLPHYFAMDYWGQG |
| | | TSVTVSSASTKGPSVFPLAPSSKSTSGGTAALGCLVK |
| | | DYFPEPVTVSWNSGALTSGVHTFPAVLQSSGLYSLSS |
| | | VVTVPSSSLGTQTYICNVNHKPSNTKVDKRVEPKSCD |
| | | KTHTCPPCPAPELLGGPSVFLFPPKPKDTLMISRTPE |
| | | VTCVVVDVSHEDPEVKFNWYVDGVEVHNAKTKPREEQ |
| | | YNSTYRVVSVLTVLHQDWLNGKEYKCKVSNKALPAPI |
| | | EKTISKAKGQPREPQVYTLPPSREEMTKNQVSLTCLV |
| | | KGFYPSDIAVEWESNGQPENNYKTTPPVLDSDGSFFL |
| | | YSKLTVDKSRWQQGNVFSCSVMHEALHNHYTQKSLSL |
| | | SPG |
| | | (SEQ ID NO: 159) |
| Chimeric | IgK light | (SEQ ID NO: 156) |
| 723C2 in | Chain | |
| IgG1KO | | |

(723-IgG1KO)

| | | |
|---|---|---|
| | | (SEQ ID NO: 157) |
| | IgG1KO heavy | GAAGTGCAGCTGGTGGAATCTGGCGGAGGACTTGTTC |
| | Chain | AACCTGGCGGCAGCCTGAAACTGTCTTGTGCCGCCAG |
| | | CGGCTTCACCTTCAGCGACTACTACATGAGCTGGGTC |
| | | CGACAGACCCCTGAGAAGAGACTGGAATGGGTCGCCT |
| | | ACATCAGCTCTGGCGGCGGAAGCAGCTACTACCCTGA |
| | | TAGCGTGAAGGGCAGATTCACCATCAGCCGGGACAAC |
| | | ACCAAGAACACCCTGTACCTGCAGATGTCCAGCCTGA |
| | | AGTCTGAGGACACCGCCGTGTACTACTGTGCCAGACT |
| | | GCCTCACTACTTCGCCATGGATTGTTGGGGCCAGGGC |
| | | ACATCTGTGACCGTTAGTTCTGCCTCCACCAAGGGCC |
| | | CATCGGTCTTCCCGCTAGCACCCTCCTCCAAGAGCAC |
| | | CTCTGGGGGCACAGCGGCCCTGGGCTGCCTGGTCAAG |
| | | GACTACTTCCCCGAACCGGTGACGGTGTCGTGGAACT |
| | | CAGGCGCCCTGACCAGCGGCGTGCACACCTTCCCGGC |
| | | TGTCCTACAGTCCTCAGGACTCTACTCCCTCAGCAGC |
| | | GTGGTGACCGTGCCCTCCAGCAGCTTGGGCACCCAGA |
| | | CCTACATCTGCAACGTGAATCACAAGCCCAGCAACAC |
| | | CAAGGTGGACAAGCGCGTTGAGCCCAAATCTTGTGAC |
| | | AAAACTCACACATGCCCACCGTGCCCAGCACCTGAAG |
| | | CCGCTGGGGGACCGTCAGTCTTCCTCTTCCCCCCAAA |
| | | ACCCAAGGACACCCTCATGATCTCCCGGACCCCTGAG |
| | | GTCACATGCGTGGTGGTGGACGTGAGCCACGAAGACC |
| | | CTGAGGTCAAGTTCAACTGGTACGTGGACGGCGTGGA |
| | | GGTGCATAATGCCAAGACAAAGCCGCGGGAGGAGCAG |
| | | TACAACAGCACGTACCGTGTGGTCAGCGTCCTCACCG |
| | | TCCTGCACCAGGACTGGCTGAATGGCAAGGAGTACAA |
| | | GTGCAAGGTCTCCAACAAAGCCCTCCCAGCCCCCATC |
| | | GAGAAAACCATCTCCAAAGCCAAAGGGCAGCCCCGAG |
| | | AACCACAGGTGTACACCCTGCCCCCATCCCGCGAGGA |
| | | GATGACCAAGAACCAGGTAAGTTTGACCTGCCTGGTC |
| | | AAAGGCTTCTATCCCAGCGACATCGCCGTGGAGTGGG |
| | | AGAGCAATGGGCAGCCGGAGAACAACTACAAGACCAC |
| | | GCCTCCCGTGCTGGACTCCGACGGCTCCTTCTTCCTC |
| | | TATAGCAAGCTCACCGTGGACAAGAGCAGGTGGCAGC |
| | | AGGGGAACGTCTTCTCATGCTCCGTGATGCATGAGGC |
| | | TCTGCACAACCACTACACGCAGAAGAGCCTCTCCCTG |
| | | TCTCCGGGT |
| | | (SEQ ID NO: 160) |
| | | EVQLVESGGGLVQPGGSLKLSCAASGFTFSDYYMSWV |
| | | RQTPEKRLEWVAYISSGGGSSYYPDSVKGRFTISRDN |
| | | TKNTLYLQMSSLKSEDTAVYYCARLPHYFAMDCWGQG |
| | | TSVTVSSASTKGPSVFPLAPSSKSTSGGTAALGCLVK |
| | | DYFPEPVTVSWNSGALTSGVHTFPAVLQSSGLYSLSS |
| | | VVTVPSSSLGTQTYICNVNHKPSNTKVDKRVEPKSCD |
| | | KTHTCPPCPAPEAAGGPSVFLFPPKPKDTLMISRTPE |
| | | VTCVVVDVSHEDPEVKFNWYVDGVEVHNAKTKPREEQ |
| | | YNSTYRVVSVLTVLHQDWLNGKEYKCKVSNKALPAPI |
| | | EKTISKAKGQPREPQVYTLPPSREEMTKNQVSLTCLV |
| | | KGFYPSDIAVEWESNGQPENNYKTTPPVLDSDGSFFL |
| | | YSKLTVDKSRWQQGNVFSCSVMHEALHNHYTQKSLSL |
| | | SPG |
| | | (SEQ ID NO: 161) |
| Chimeric 723C2 | IgK in light | (SEQ ID NO: 156) |
| in IgG4Pro | Chain | |
| (723-IgG4Pro) | | |
| | | (SEQ ID NO: 157) |
| | IgG4Pro | GAAGTGCAGCTGGTGGAATCTGGCGGAGGACTTGTTC |
| | heavy | AACCTGGCGGCAGCCTGAAACTGTCTTGTGCCGCCAG |
| | Chain | CGGCTTCACCTTCAGCGACTACTACATGAGCTGGGTC |
| | | CGACAGACCCCTGAGAAGAGACTGGAATGGGTCGCCT |
| | | ACATCAGCTCTGGCGGCGGAAGCAGCTACTACCCTGA |
| | | TAGCGTGAAGGGCAGATTCACCATCAGCCGGGACAAC |
| | | ACCAAGAACACCCTGTACCTGCAGATGTCCAGCCTGA |
| | | AGTCTGAGGACACCGCCGTGTACTACTGTGCCAGACT |
| | | GCCTCACTACTTCGCCATGGATTATTGGGGCCAGGGC |
| | | ACCAGCGTGACCGTTTCTTCTGCCTCCACAAAGGGCC |
| | | CTTCCGTGTTCCCCCTGGCCCCTTGCTCCCGGTCCAC |
| | | CTCCGAGTCTACCGCCGCTCTGGGCTGCCTGGTCAAG |
| | | GACTACTTCCCCGAGCCCGTGACCGTGTCCTGGAACT |
| | | CTGGCGCCCTGACCTCCGGCGTGCACACCTTCCCTGC |
| | | TGTGCTGCAGTCCTCCGGCCTGTACTCCCTGTCCTCC |
| | | GTCGTGACCGTGCCCTCCTCTAGCCTGGGCACCAAGA |
| | | CCTACACCTGTAACGTGGACCACAAGCCCTCCAACAC |
| | | CAAGGTGGACAAGCGGGTGGAATCTAAGTACGGCCCT |
| | | CCCTGCCCCCCCTGCCCTGCCCCTGAATTTCTGGGCG |
| | | GACCCTCCGTGTTCCTGTTCCCCCCAAAGCCCAAGGA |
| | | CACCCTGATGATCTCCCGGACCCCCGAAGTGACCTGC |
| | | GTGGTGGTGGACGTGTCCCAGGAAGATCCCGAGGTCC |
| | | AGTTTAATTGGTACGTGGACGGCGTGGAAGTGCACAA |
| | | CGCCAAGACCAAGCCCAGAGAGGAACAGTTCAACTCC |
| | | ACCTACCGGGTGGTGTCCGTGCTGACCGTGCTGCACC |
| | | AGGACTGGCTGAACGGCAAAGAGTACAAGTGCAAGGT |
| | | GTCCAACAAGGGCCTGCCCTCCAGCATCGAAAAGACC |
| | | ATCTCCAAGGCCAAGGGCCAGCCCCGCGAGCCCCAGG |
| | | TGTACACCCTGCCTCCAAGCCAGGAAGAGATGACCAA |
| | | GAACCAGGTGTCCCTGACCTGTCTGGTCAAGGGCTTC |
| | | TACCCCTCCGATATCGCCGTGGAATGGGAGTCCAACG |
| | | GCCAGCCCGAGAACAACTACAAGACCACCCCCCCTGT |
| | | GCTGGACTCCGACGGCTCCTTCTTCCTGTACTCTCGG |
| | | CTGACCGTGGACAAGTCCCGGTGGCAGGAAGGCAACG |
| | | TCTTCTCCTGCTCCGTGATGCACGAGGCCCTGCACAA |
| | | CCACTACACCCAGAAGTCCCTGTCCCTGAGCCTGGGC |
| | | (SEQ ID NO: 162) |
| | | EVQLVESGGGLVQPGGSLKLSCAASGFTFSDYYMSWV |
| | | RQTPEKRLEWVAYISSGGGSSYYPDSVKGRFTISRDN |
| | | TKNTLYLQMSSLKSEDTAVYYCARLPHYFAMDYWGQG |
| | | TSVTVSSASTKGPSVFPLAPCSRSTSESTAALGCLVK |
| | | DYFPEPVTVSWNSGALTSGVHTFPAVLQSSGLYSLSS |
| | | VVTVPSSSLGTKTYTCNVDHKPSNTKVDKRVESKYGP |
| | | PCPPCPAPEFLGGPSVFLFPPKPKDTLMISRTPEVTC |
| | | VVVDVSQEDPEVQFNWYVDGVEVHNAKTKPREEQFNS |
| | | TYRVVSVLTVLHQDWLNGKEYKCKVSNKGLPSSI |
| | | EKTISKAKGQPREPQVYTLPPSQEEMTKNQVSLTCLV |
| | | KGFYPSDIAVEWESNGQPENNYKTTPPVLDSDGSFFL |
| | | YSRLTVDKSRWQEGNVFSCSVMHEALHNHYTQKSLSL |
| | | SLG |
| | | (SEQ ID NO: 163) |

The amino acids corresponding to the change from DC to DY in H-CDR3 are underlined in the amino acid sequences in Table 8.

### Humanization and Amino Acid Sequence Variants

Further variant anti-PD-1 antibodies and antibody fragments can be engineered based on the set of CDRs depicted in Tables 3 and 4. It is to be understood that in the variant anti-PD-1 antibodies and antibody fragments the amino acid sequence of the CDRs remain unchanged but the surrounding regions, e.g., FR regions can be engineered. Amino acid sequence variants of the anti-PD-1 antibody can be prepared by introducing appropriate nucleotide changes into the anti-PD-1 antibody DNA, or by peptide synthesis. Such variants include, for example, deletions from, and/or insertions into and/or substitutions of, residues within the amino acid sequences of the anti-PD-1 antibodies of the examples herein. Any combination of deletions, insertions, and substitutions is made to arrive at the final construct, provided that the final construct possesses the desired characteristics. The amino acid changes also may alter post-translational processes of the humanized or variant anti-PD-1 antibody, such as changing the number or position of glycosylation sites.

In some embodiments, the present invention includes anti-PD-1-antibodies or antibody fragments thereof having a variable heavy chain and a variable light chain, wherein the variable heavy chain amino acid sequence and the variable light chain amino acid sequence are at least 80%, at least 85%, at least 90%, at least 92.5%, at least 95%, at least 98%, or at least 99% identical to the amino acid sequences disclosed in Tables 1, 2, 5 and 6.

In some embodiments, the present invention includes anti-PD-1-antibodies or antibody fragments thereof having a variable heavy chain and a variable light chain, wherein the variable heavy chain amino acid sequence and the variable light chain amino acid sequence are at least 80%, at least 85%, at least 90%, at least 92.5%, at least 95%, at least 98%, or at least 99% identical to the amino acid sequences of SEQ ID NOs. 131, 133, 135, 137 or 139, and SEQ ID NOs. 125, 127 or 129, respectively.

In some embodiments, the present invention includes anti-PD-1 antibodies having a heavy chain and a light chain, wherein the heavy chain amino acid sequence and the light chain amino acid sequence are at least 95%, at least 98%, or at least 99% identical to the amino acid sequences disclosed in Tables 7 and 8.

Another type of amino acid variant of the antibody involves altering the original glycosylation pattern of the antibody. The term “altering” in this context means deleting one or more carbohydrate moieties found in the antibody, and/or adding one or more glycosylation sites that were not previously present in the antibody. For example, an antibody may comprise an amino acid substitution at position 297 of the human IgG1 heavy chain to abrogate oligosaccharyltransferase enzyme complex-mediated glycosylation by replacing the asparagine 297 (e.g. N297A, N297G).

In some aspects, the present invention includes nucleic acid molecules that encode the amino acid sequence variants of the anti-PD-1 antibodies described herein. Nucleic acid molecules encoding amino acid sequence variants of an anti-PD-1 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-PD-1 antibody. For example, nucleic acid molecules according to the invention also encompass nucleic acid molecules which hybridize under stringent conditions to nucleic acid molecules as disclosed herein, whereby the term “stringent conditions” within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C.

In certain embodiments, the anti-PD-1 antibody is an antibody fragment. There are techniques that have been developed for the production of antibody fragments. Fragments can be derived via proteolytic digestion of intact antibodies (see, e.g., Morimoto et al., 1992, Journal of Biochemical and Biophysical Methods 24:107-117; and Brennan et al., 1985, Science 229:81). Alternatively, the fragments can be produced directly in recombinant host cells. For example, Fab′-SH fragments can be directly recovered from E. coli and chemically coupled to form F(ab′)₂ fragments (see, e.g., Carter et al., 1992, Bio/Technology 10:163-167). By another approach, F(ab′)₂ fragments can be isolated directly from recombinant host cell culture. Other techniques for the production of antibody fragments will be apparent to the skilled practitioner.

In one aspect, the anti-PD-1 antibodies and antigen-binding fragments thereof can include modifications, such as glycosylation or deamidation.

In certain embodiments, it may be desirable to use an anti-PD-1 antibody fragment, rather than an intact antibody. It may be desirable to modify the antibody fragment in order to increase its serum half-life. This can be achieved, for example, by incorporation of a salvage receptor binding epitope into the antibody fragment. In one method, the appropriate region of the antibody fragment can be altered (e.g., mutated), or the epitope can be incorporated into a peptide tag that is then fused to the antibody fragment at either end or in the middle, for example, by DNA or peptide synthesis. See, e.g., WO 96/32478. For example, antibody fragments of the invention may also be fused to human serum albumin to increase the serum half-life, if the use of a full-length IgG1 scaffold is undesirable. Such fusion proteins of the antibody fragment with human serum albumin may be advantageous in situations in which two different antibody fragments need to be fused to increase avidity, or to generate a bispecific binding protein with extended serum half-life (see e.g. WO05077042 A2).

In other embodiments, the present invention includes covalent modifications of the anti-PD-1 antibodies. Covalent modifications include modification of cysteinyl residues, histidyl residues, lysinyl and amino-terminal residues, arginyl residues, tyrosyl residues, carboxyl side groups (aspartyl or glutamyl), glutaminyl and asparaginyl residues, or seryl, or threonyl residues. Another type of covalent modification involves chemically or enzymatically coupling glycosides to the antibody. Such modifications may be made by chemical synthesis or by enzymatic or chemical cleavage of the antibody, if applicable. Other types of covalent modifications of the antibody can be introduced into the molecule by reacting targeted amino acid residues of the antibody with an organic derivatizing agent that is capable of reacting with selected side chains or the amino- or carboxy-terminal residues.

Removal of any carbohydrate moieties present on the antibody can be accomplished chemically or enzymatically. Chemical deglycosylation is described by Hakimuddin et al., 1987, Arch. Biochem. Biophys. 259:52 and by Edge et al., 1981, Anal. Biochem., 118:131. Enzymatic cleavage of carbohydrate moieties on antibodies can be achieved by the use of a variety of endo- and exo-glycosidases as described by Thotakura et al., 1987, Meth. Enzymol 138:350.

Another type of useful covalent modification comprises linking the antibody to one of a variety of nonproteinaceous polymers, e.g., polyethylene glycol, polypropylene glycol, or polyoxyalkylenes, in the manner set forth in one or more of U.S. Pat. Nos. 4,640,835, 4,496,689, 4,301,144, 4,670,417, 4,791,192 and 4,179,337.

### Epitope Binding

In another aspect, the invention relates to an antibody or antigen-binding fragment thereof that recognizes a specific “PD-1 antigen epitope” and “PD-1 epitope”.

As used herein, the terms “PD-1 antigen epitope” and “PD-1 epitope” refer to a molecule (e.g., a peptide) or a fragment of a molecule capable of binding to an anti-PD-1 antibody or antigen-binding fragment thereof. These terms further include, for example, a PD-1 antigenic determinant recognized by any of the antibodies or antibody fragments of the present invention.

PD-1 antigen epitopes can be included in proteins, protein fragments, peptides or the like. The epitopes are most commonly proteins, short oligopeptides, oligopeptide mimics (i.e., organic compounds that mimic antibody binding properties of the PD-1 antigen), or combinations thereof.

In one aspect, an anti-PD-1 antibody of the present invention or antigen-binding fragment thereof binds specifically to a PD-1 epitope in a manner that mimics the binding of the physiological ligand resulting in antibody-mediated agonism.

The present invention also provides an anti-PD-1 antibody or antigen-binding fragment thereof that competes for binding to PD-1 with an anti-PD-1 antibody according to the present invention. In one embodiment, the present invention provides an anti-PD-1 antibody or antigen-binding fragment thereof that competes for binding to PD-1 with any one of Antibody A, Antibody B, Antibody C, Antibody D or Antibody E described herein. Competition assays may be conducted for example as described in PLOS One. 2014; 9 (3): e92451 using a biosensor, or PLOS One 2020 Mar. 5;15 (3): e0229206, or by a method disclosed herein.

### Therapeutic Uses

In one embodiment, the anti-PD-1 antibodies of the invention or antigen-binding fragments thereof are useful for treating or preventing PD-1 pathway disorders.

In another embodiment, the anti-PD-1 antibodies of the invention or antigen-binding fragments thereof are useful as a medicament.

Accordingly, in one embodiment, the present invention provides a method of modulating the interaction between PD-1 and PD-L1 in a human patient comprising administering to said human patient a composition comprising an anti-PD-1 antibody or antigen-binding fragment thereof according to the present invention in an amount sufficient to activate the PD-1 pathway in said human patient. In one embodiment, the present invention provides an anti-PD-1 antibody or antigen-binding fragment thereof according to the present invention for use in modulating the interaction between PD-1 and PD-L1 in a human patient. In one embodiment, the present invention provides the use of an anti-PD-1 antibody or antigen-binding fragment thereof according to the present invention in the manufacture of a medicament for modulating the interaction between PD-1 and PD-L1 in a human patient.

In one embodiment, the present invention provides a method of attenuating PD-1 expressing T cell activity in a human patient comprising administering to said human patient a composition comprising an anti-PD-1 antibody or antigen-binding fragment thereof according to the present invention in an amount sufficient to down-modulate an immune response in said human patient. In one embodiment, the present invention provides an anti-PD-1 antibody or antigen-binding fragment thereof according to the present invention for use in attenuating PD-1 expressing T cell activity in a human patient. In one embodiment, the present invention provides the use of an anti-PD-1 antibody or antigen-binding fragment thereof according to the present invention in the manufacture of a medicament for attenuating PD-1 expressing T cell activity in a human patient.

In one embodiment, a PD-1 pathway disease or disorder is systemic sclerosis (SSc), systemic lupus erythematosus, polymyositis, giant cell arteritis, psoriasis, psoriatic arthritis, ankylosing spondylitis or inflammatory bowel disease. Accordingly, in one embodiment, the present invention provides a method of treating or preventing systemic sclerosis (SSc), systemic lupus erythematosus, polymyositis, giant cell arteritis, psoriasis, psoriatic arthritis, ankylosing spondylitis or inflammatory bowel disease in a human patient comprising administering to said human patient a composition comprising an anti-PD-1 antibody or antigen-binding fragment thereof according to the present invention. In one embodiment, the present invention provides an anti-PD-1 antibody or antigen-binding fragment thereof according to the present invention for use in treating or preventing systemic sclerosis (SSc), systemic lupus erythematosus, polymyositis, giant cell arteritis, psoriasis, psoriatic arthritis, ankylosing spondylitis or inflammatory bowel disease in a human patient. In one embodiment, the present invention provides the use of an anti-PD-1 antibody or antigen-binding fragment thereof according to the present invention in the manufacture of a medicament for treating or preventing systemic sclerosis (SSc), systemic lupus erythematosus, polymyositis, giant cell arteritis, psoriasis, psoriatic arthritis, ankylosing spondylitis or inflammatory bowel disease in a human patient.

In one embodiment, a PD-1 pathway disease or disorder is chronic or acute, such as chronic inflammatory disease or acute inflammatory disease. In one embodiment, a PD-1 pathway disease or disorder is arthritis, rheumatoid arthritis, asthma, COPD, pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, psoriatic arthritis, vasculitis, surgical adhesions, stroke, Type I Diabetes, Lyme disease, meningoencephalitis, autoimmune uveitis, multiple sclerosis, lupus (such as systemic lupus erythematosus), Guillain-Barr syndrome, Atopic dermatitis, autoimmune hepatitis, fibrosing alveolitis, Grave's disease, IgA nephropathy, idiopathic thrombocytopenia purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease, transplant rejection, heart disease including ischaemic diseases such as myocardial infarction as well as atherosclerosis, intravascular coagulation, bone resorption, osteoporosis, osteoarthritis, periodontitis and hypochlorhydia, infertility related to lack of fetal-maternal tolerance, Sjogren's Syndrome, vitiligo, myasthenia gravis or systemic sclerosis.

Accordingly, in one embodiment, the present invention provides a method of treating or preventing one of the above diseases or disorders in a human patient comprising administering to said human patient a composition comprising an anti-PD-1 antibody or antigen-binding fragment thereof according to the present invention. In one embodiment, the present invention provides an anti-PD-1 antibody or antigen-binding fragment thereof according to the present invention for use in treating or preventing one of the above diseases or disorders in a human patient. In one embodiment, the present invention provides the use of an anti-PD-1 antibody or antigen-binding fragment thereof according to the present invention in the manufacture of a medicament for treating or preventing one of the above diseases or disorders in a human patient.

In one aspect, the PD-1 antibody or antigen-binding fragment thereof for use or in a use as described above or in a method as described above is an agonist anti-PD-1 antibody or antigen-binding fragment thereof.

### Non-Therapeutic Uses

The antibodies described herein are useful as affinity purification agents. In this process, the antibodies are immobilized on a solid phase such a Protein A resin, using methods well known in the art. The immobilized antibody is contacted with a sample containing the PD-1 protein (or fragment thereof) to be purified, and thereafter the support is washed with a suitable solvent that will remove substantially all the material in the sample except the PD-1 protein, which is bound to the immobilized antibody. Finally, the support is washed with another suitable solvent that will release the PD-1 protein from the antibody.

The anti-PD-1 antibodies and fragments thereof of the invention as disclosed herein are also useful in diagnostic assays to detect and/or quantify PD-1 protein, for example, detecting PD-1 expression in specific cells, tissues, or serum.

It will be advantageous in some embodiments, for example, for diagnostic purposes to label the antibody with a detectable detectable moiety. Numerous detectable labels are available, including radioisotopes, fluorescent labels, enzyme substrate labels, quantum dots and the like. The label may be indirectly conjugated with the antibody using various known techniques. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody can be conjugated with a small hapten (such as digoxin) and one of the different types of labels mentioned above is conjugated with an anti-hapten antibody (e.g., anti-digoxin antibody). Thus, indirect conjugation of the label with the antibody can be achieved.

Exemplary radioisotopes labels include 35S, 14C, 1251, 3H, and 1311. The antibody can be labeled with the radioisotope, using the techniques described in, for example, Current Protocols in Immunology, Volumes 1 and 2, 1991, Coligen et al., Ed. Wiley-Interscience, New York, N.Y., Pubs. Radioactivity can be measured, for example, by scintillation counting.

Exemplary fluorescent labels include labels derived from rare earth chelates (europium chelates) or fluorescein and its derivatives, rhodamine and its derivatives, dansyl, Lissamine, phycoerythrin, and Texas Red are available, or e.g. any of the following fluorescent labels: dialkylaminocoumarin, rhodamine isothiocyanate, Alexa 350, Alexa 430, Alexa Fluor 488, Alexa Fluor 532, Alexa Fluor 546, Alexa Fluor 568, Alexa Fluor 594, Alexa Fluor 633, Alexa Fluor 660, Alexa Fluor 680, AMCA, aminoacridine, BODIPY 630/650, BODIPY 650/665, BODIPY-FL, BODIPY-R6G, BODIPY-TMR, BODIPY-TRX, BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin. DM-NERF, Eosin, Erythrosin, Fluorescein, FA, Hydroxycoumarin, IRDyes (IRD40, IRD 700, IRD 800), JOE, Lissamine rhodamine B, Marina Blue, Methoxy coumarin, Naphtho fluorescein, Oregon Green 488, Oregon Green 500, Oregon Green 514, Pacific Blue, PyMPO, 5-carboxy-4′,5,-dichloro-2′,7′-dimethoxy fluorescein, 5-carboxy-2′,4′,5,7′-tetrachlorofluorescein, 5-carboxyfluorescein, 5-carboxyrhodamine, 6-carboxyrhodamine, 6-carboxytetramethyl amino, Cascade Blue, Cy2, Cy3, Cy5,6-FAM, dansyl chloride, fluorescein, HEX, 6-JOE, NBD (7-nitrobenz-2-oxa-1,3-diazole), Oregon Green 488, Oregon Green 500, Oregon Green 514, Pacific Blue, phthalic acid, terephthalic acid, isophthalic acid, cresyl fast violet, cresyl blue violet, brilliant cresyl blue, para-aminobenzoic acid, erythrosine, phthalocyanines, azomethines, cyanines, xanthines, succinylfluoresceins, rare earth metal cryptates, europium trisbipyridine diamine, a europium cryptate or chelate, diamine, dicyanins, La Jolla blue dye, aUopycocyanin, allococyanin B, phycocyanin C, phycocyanin R, thiamine, phycoerythrocyanin, phycoerythrin R, REG, Rhodamine Green, rhodamine isothiocyanate, Rhodamine Red, TAMRA, TET, TRIT (tetramethyl rhodamine isothiol), Tetramethylrhodamine, or Texas Red. The fluorescent labels can be conjugated to the antibody via known techniques, such as those disclosed in Current Protocols in Immunology, supra, for example. Fluorescence can be quantified using a fluorimeter.

There are various well-characterized enzyme-substrate labels known in the art (see, e.g., U.S. Pat. No. 4,275,149 for a review). The enzyme generally catalyzes a chemical alteration of the chromogenic substrate that can be measured using various techniques. For example, alteration may be a color change in a substrate that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured, using a chemiluminometer, for example, or donates energy to a fluorescent acceptor.

Examples of enzymatic labels include luciferases such as firefly luciferase and bacterial luciferase (U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (such as glucose oxidase, galactose oxidase, and glucose-6-phosphate dehydrogenase), heterocydic oxidases (such as uricase and xanthine oxidase), lactoperoxidase, microperoxidase, and the like. Techniques for conjugating enzymes to antibodies are described, for example, in O'Sullivan et al., 1981, Methods for the Preparation of Enzyme-Antibody Conjugates for use in Enzyme Immunoassay, in Methods in Enzym. (J. Langone & H. Van Vunakis, eds.), Academic press, N.Y., 73:147-166.

Examples of enzyme-substrate combinations include, for example: Horseradish peroxidase (HRPO) with hydrogen peroxidase as a substrate, wherein the hydrogen peroxidase oxidizes a dye precursor such as orthophenylene diamine (OPD) or 3,3′,5,5′-tetramethyl benzidine hydrochloride (TMB); alkaline phosphatase (AP) with para-Nitrophenyl phosphate as chromogenic substrate; and β-D-galactosidase (β-D-Gal) with a chromogenic substrate such as p-nitrophenyl-β-D-galactosidase or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

In another embodiment, an anti-PD-1 antibody or antibody fragment of the invention is used unlabeled and detected with a labeled antibody that binds the anti-PD-1 antibody or fragment thereof. For example, labeled anti-human Fc, or anti-human Fab antibodies may be used to detect the unlabeled anti-PD-1 antibody or fragment. The use of an unlabeled anti-PD-1 antibody or fragments thereof according to the invention may be advantageous to achieve a better tissue penetration, as the fluorescent label will increase the molecular weight and/or increase hydrophobicity of the antibody or antibody fragment it is fused to thereby reducing tissue penetration.

The antibodies described herein may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987). Diagnostic Kits.

A humanized anti-PD-1 antibody of the invention can be used in a diagnostic kit, i.e., a packaged combination of reagents in predetermined amounts with instructions for performing the diagnostic assay. Where the antibody is labeled with an enzyme, the kit may include substrates and cofactors required by the enzyme such as a substrate precursor that provides the detectable chromophore or fluorophore. In addition, other additives may be included such as stabilizers, buffers (for example a block buffer or lysis buffer), and the like. The relative amounts of the various reagents may be varied widely to provide for concentrations in solution of the reagents that substantially optimize the sensitivity of the assay. The reagents may be provided as dry powders, usually lyophilized, including excipients that on dissolution will provide a reagent solution having the appropriate concentration.

### Diagnostic Kits

An anti-PD-1 antibody or fragment thereof can be used in a diagnostic kit, i.e., a packaged combination of reagents in predetermined amounts with instructions for performing the diagnostic assay. Where the antibody is labeled with an enzyme, the kit may include substrates and cofactors required by the enzyme such as a substrate precursor that provides the detectable chromophore or fluorophore. In addition, other additives may be included such as stabilizers, buffers (for example a block buffer or lysis buffer), and the like. The relative amounts of the various reagents may be varied widely to provide for concentrations in solution of the reagents that substantially optimize the sensitivity of the assay. The reagents may be provided as dry powders, usually lyophilized, including excipients that on dissolution will provide a reagent solution having the appropriate concentration.

### Compositions and Administration Thereof

A composition comprising an anti-PD-1 antibody or an antigen-binding fragment thereof according to the invention can be administered to a subject having or at risk of the PD-1 pathway diseases or disorders described herein. The invention further provides for the use of an anti-PD-1 antibody or an antigen-binding fragment thereof in the manufacture of a medicament for prevention or treatment of a PD-1 pathway disease or disorder. The term “subject” as used herein means any mammalian patient to which an anti-PD-1 antibody or an antigen-binding fragment thereof can be administered, including, e.g., humans and certain non-human mammals, such as primates, and dogs. Subjects specifically intended for treatment using the methods described herein include humans. The anti-PD-1 antibody or an antigen-binding fragment thereof of the invention can be administered either alone or in combination with other compositions.

In one aspect, the present invention also provides pharmaceutical compositions comprising an antibody or antigen-binding fragment thereof of the present invention.

Various delivery systems are known and can be used to administer the anti-PD-1 antibody or an antigen-binding fragment thereof. Methods of introduction include but are not limited to intravitreal, eye drops, intradermal, intramuscular, intraperitoneal, intravenous, subcutaneous, intranasal, epidural, and oral routes. The anti-PD-1 antibody or an antigen-binding fragment thereof can be administered, for example by infusion, bolus or injection, and can be administered together with other biologically active agents. Administration can be systemic or local. Formulations for such injections may be prepared in, for example, prefilled syringes.

An anti-PD-1 antibody or an antigen-binding fragment thereof can be administered as pharmaceutical compositions comprising a therapeutically effective amount of the anti-PD-1 antibody or an antigen-binding fragment thereof and one or more pharmaceutically compatible ingredients.

In typical embodiments, the pharmaceutical composition is formulated in accordance with routine procedures as a pharmaceutical composition adapted for intravenous or subcutaneous administration to human beings. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing an anti-PD-1 antibody or an antigen-binding fragment thereof in lyophilized form and (b) a second container containing a pharmaceutically acceptable diluent (e.g., sterile water) for injection. The pharmaceutically acceptable diluent can be used for reconstitution or dilution of the lyophilized anti-PD-1 antibody or antigen-binding fragment thereof. Optionally associated with such container(s) can be a notice in the form prescribed by a governmental agency regulating the manufacture, use or sale of pharmaceuticals or biological products, which notice reflects approval by the agency of manufacture, use or sale for human administration.

The amount of the anti-PD-1 antibody or antigen-binding fragment thereof that is effective in the treatment or prevention PD-1 pathway diseases or disorders can be determined by standard clinical techniques. In addition, in vitro assays may optionally be employed to help identify optimal dosage ranges. The precise dose to be employed in the formulation will also depend on the route of administration, and the stage of disorder, and should be decided according to the judgment of the practitioner and each patient's circumstances. Effective doses may be extrapolated from dose-response curves derived from in vitro or animal model test systems.

For example, toxicity and therapeutic efficacy of the anti-PD-1 antibody or antigen-binding fragment thereof can be determined in cell cultures or experimental animals by standard pharmaceutical procedures for determining the ED₅₀ (the dose therapeutically effective in 50% of the population). An anti-PD-1 antibody or antigen-binding fragment thereof that exhibits a large therapeutic index is preferred.

The data obtained from the cell culture assays and animal studies can be used in formulating a range of dosage for use in humans. The dosage of the anti-PD-1 antibody or antigen-binding fragment thereof typically lies within a range of circulating concentrations that include the ED₅₀ with little or no toxicity. The dosage may vary within this range depending upon the dosage form employed and the route of administration utilized. For any anti-PD-1 antibody or antigen-binding fragment thereof used in the method, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography, ELISA and the like.

In one embodiment, the anti-PD-1-antibody is administered at regular intervals.

In some embodiments, antibodies of the present invention can be formulated to doses, which include for example from 1 mg/ml to 250 mg/ml, for example from 20 mg/ml to 200 mg/ml.

In some embodiments, the pharmaceutical compositions comprising the anti-PD-1 antibody or antigen-binding fragment thereof can further comprise a therapeutic agent, either conjugated or unconjugated to the binding agent.

Such combination therapy administration can have an additive or synergistic effect on disease parameters (e.g., severity of a symptom, the number of symptoms, or frequency of relapse).

With respect to therapeutic regimens for combinatorial administration, in a specific embodiment, an anti-PD-1 antibody or antigen-binding fragment thereof is administered concurrently with a therapeutic agent. In another specific embodiment, the therapeutic agent is administered prior or subsequent to administration of the anti-PD-1 antibody or antigen-binding fragment thereof.

### Polynucleotides, Vectors, Host Cells, and Recombinant Methods

The present invention relates to isolated polynucleotides that comprise a sequence encoding an anti-PD-1 antibody or antigen-binding fragment thereof, vectors, and host cells comprising the polynucleotides, and recombinant techniques for production of the antibody. The isolated polynucleotides can encode any desired form of the anti-PD-1 antibody including, for example, full length monoclonal antibodies, Fab, Fab′, F(ab′)₂, and Fv fragments, diabodies, linear antibodies, single-chain antibody molecules, and multispecific antibodies formed from antibody fragments.

The polynucleotide(s) that comprise a sequence encoding an anti-PD-1 antibody or a fragment or chain thereof can be fused to one or more regulatory or control sequence, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The anti-PD-1 antibodies can also be produced as fusion polypeptides, in which the antibody is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the host cell. For prokaryotic host cells that do not recognize and process the anti-PD-1 antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO90/13646. In mammalian cells, mammalian signal sequences as well as viral secretory leaders, for example, the herpes simplex gD signal, can be used. The DNA for such precursor region is ligated in reading frame to DNA encoding the humanized anti-PD-1 antibody.

Expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2-v. plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid, and hygromycin. Common selectable markers for mammalian cells are those that enable the identification of cells competent to take up a nucleic acid encoding a humanized anti-PD-1 antibody, such as DHFR (dihydrofolate reductase), thymidine kinase, metallothionein-and-II (such as primate metallothionein genes), adenosine deaminase, ornithine decarboxylase, and the like. Cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., DG44).

Alternatively, host cells (particularly wild-type hosts that contain endogenous DHFR) transformed or co-transformed with DNA sequences encoding anti-PD-1 antibody, wild-type DHFR protein, and another selectable marker such as aminoglycoside 3′-phosphotransferase (APH), can be selected by cell growth in medium containing a selection agent for the selectable marker such as an aminoglycosidic antibiotic, e.g., kanamycin, neomycin, or G418. See, e.g., U.S. Pat. No. 4,965,199.

Where the recombinant production is performed in a yeast cell as a host cell, the TRP1 gene present in the yeast plasmid YRp7 (Stinchcomb et al., 1979, Nature 282:39) can be used as a selectable marker. The TRP1 gene provides a selection marker for a mutant strain of yeast lacking the ability to grow in tryptophan, for example, ATCC No. 44076 or PEP4-1 (Jones, 1977, Genetics 85:12). The presence of the trp1 lesion in the yeast host cell genome then provides an effective environment for detecting transformation by growth in the absence of tryptophan. Similarly, Leu2p-deficient yeast strains such as ATCC 20,622 and 38,626 are complemented by known plasmids bearing the LEU2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis (Van den Berg, 1990, Bio/Technology 8:135). Stable multi-copy expression vectors for secretion of mature recombinant human serum albumin by industrial strains of Kluyveromyces have also been disclosed (Fleer et al., 1991, Bio/Technology 9:968-975).

Expression and cloning vectors usually contain a promoter that is recognized by the host organism and is operably linked to the nucleic acid molecule encoding an anti-PD-1 antibody or polypeptide chain thereof. Promoters suitable for use with prokaryotic hosts include phoA promoter, β-lactamase and lactose promoter systems, alkaline phosphatase, tryptophan (trp) promoter system, and hybrid promoters such as the tac promoter. Other known bacterial promoters are also suitable. Promoters for use in bacterial systems also will contain a Shine-Dalgamo (S.D.) sequence operably linked to the DNA encoding the humanized anti-PD-1 antibody.

Many eukaryotic promoter sequences are known. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Inducible promoters have the additional advantage of transcription controlled by growth conditions. These include yeast promoter regions for alcohol dehydrogenase 2, isocytochrome C, acid phosphatase, derivative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657 or Baghban et al. Molecular Biotechnology (2019) 61:365-384. Yeast enhancers also are advantageously used with yeast promoters.

Anti-PD-1 antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, or from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a Hindlll E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., 1982, Nature 297:598-601, disclosing expression of human p-interferon cDNA in mouse cells under the control of a thymidine kinase promoter from herpes simplex virus. Alternatively, the rous sarcoma virus long terminal repeat can be used as the promoter.

Another useful element that can be used in a recombinant expression vector is an enhancer sequence, which is used to increase the transcription of a DNA encoding an anti-PD-1 antibody by higher eukaryotes. Many enhancer sequences are now known from mammalian genes (e.g., globin, elastase, albumin, α-fetoprotein, and insulin). Typically, however, an enhancer from a eukaryotic cell virus is used. Examples include the SV40 enhancer on the late side of the replication origin (bp 100-270), the cytomegalovirus early promoter enhancer, the polyoma enhancer on the late side of the replication origin, and adenovirus enhancers. See also Yaniv, 1982, Nature 297:17-18 for a description of enhancing elements for activation of eukaryotic promoters. The enhancer may be spliced into the vector at a position 5′ or 3′ to the anti-PD-1 antibody-encoding sequence, but is preferably located at a site 5′ from the promoter.

Expression vectors used in eukaryotic host cells (yeast, fungi, insect, plant, animal, human, or nucleated cells from other multicellular organisms) can also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-PD-1 antibody. One useful transcription termination component is the bovine growth hormone polyadenylation region. See WO94/11026 and the expression vector disclosed therein. In some embodiments, anti-ANGPT2 antibodies can be expressed using the CHEF system. (See, e.g., U.S. Pat. No. 5,888,809; the disclosure of which is incorporated by reference herein.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for anti-PD-1 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated anti-PD-1 antibody are derived from multicellular organisms. Examples of invertebrate cells include plant and insect cells, including, e.g., numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk worm). A variety of viral strains for transfection are publicly available, e.g., the L−1 variant of Autographa californica NPV and the Bm-5 strain of Bombyx mori NPV, and such viruses may be used, particularly for transfection of Spodoptera frugiperda cells.

Plant cell cultures of cotton, corn, potato, soybean, petunia, tomato, and tobacco can also be utilized as hosts.

The anti-PD-1 antibodies or antigen-binding fragments thereof can also be incorporated in viral vectors, i.e. the polynucleotide encoding for the anti-PD-1 antibody or antigen-binding fragment thereof is introduced into the viral vector and then expressed in the body of the patient after infection with the virus.

In another aspect, expression of the anti-PD-1 antibody or antigen-binding fragment thereof is carried out in vertebrate cells. The propagation of vertebrate cells in culture (tissue culture) has become routine procedure and techniques are widely available. Examples of useful mammalian host cell lines are monkey kidney CV1 line transformed by SV40 (COS-7, ATCC CRL 1651), human embryonic kidney line (293 or 293 cells subcloned for growth in suspension culture, (Graham et al., 1977, J. Gen Virol. 36:59), baby hamster kidney cells (BHK, ATCC CCL 10), Chinese hamster ovary cells/-DHFR1 (CHO, Urlaub et al., 1980, Proc. Natl. Acad. Sci. USA 77:4216; e.g., DG44), mouse sertoli cells (TM4, Mather, 1980, Biol. Reprod. 23:243-251), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TR1 cells (Mather et al., 1982, Annals N.Y. Acad. Sci. 383:44-68), MRC 5 cells, FS4 cells, and human hepatoma line (Hep G2).

Host cells are transformed with the above-described expression or cloning vectors for or antigen-binding fragment thereof antibody production and cultured in conventional nutrient media modified as appropriate for inducing promoters, selecting transformants, or amplifying the genes encoding the desired sequences.

The host cells used to produce an antibody or antigen-binding fragment thereof described herein may be cultured in a variety of media. Commercially available media such as Ham's F10 (Sigma-Aldrich Co., St. Louis, Mo.), Minimal Essential Medium ((MEM), (Sigma-Aldrich Co.), RPMI-1640 (Sigma-Aldrich Co.), and Dulbecco's Modified Eagle's Medium ((DMEM), Sigma-Aldrich Co.) are suitable for culturing the host cells. In addition, any of the media described in one or more of Ham et al., 1979, Meth. Enz. 58:44, Barnes et al., 1980, Anal. Biochem. 102:255, U.S. Pat. Nos. 4,767,704, 4,657,866, 4,927,762, 4,560,655, 5,122,469, WO 90/103430, and WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamicin), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, the cells may be disrupted to release protein as a first step. Particulate debris, either host cells or lysed fragments, can be removed, for example, by centrifugation or ultrafiltration. Carter et al., 1992, Bio/Technology 10:163-167 describes a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. A variety of methods can be used to isolate the antibody from the host cell.

The antibody composition prepared from the cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (see, e.g., Lindmark et al., 1983 J. Immunol. Meth. 62:1-13). Protein G is recommended for all mouse isotypes and for human gamma3 (see, e.g., Guss et al., 1986 EMBO J. 5:1567-1575). A matrix to which an affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25M salt).

Also included are nucleic acids that hybridize under low, moderate, and high stringency conditions, in particular under high stringency conditions, as defined herein, to all or a portion (e.g., the portion encoding the variable region) of the nucleotide sequence represented by isolated polynucleotide sequence(s) that encode an anti-PD-1 antibody or antibody fragment. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, or at least 98%, identical to the sequence of a portion or all of a nucleic acid encoding an anti-PD-1 polypeptide (e.g., a heavy chain or light chain variable region), or its complement. Hybridizing nucleic acids of the type described herein can be used, for example, as a cloning probe, a primer, e.g., a PCR primer, or a diagnostic probe. In one aspect, “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

In one embodiment, the present invention relates to an isolated polynucleotide comprising a nucleotide sequence encoding a heavy chain variable region comprising the amino acid sequence of any one of SEQ ID NOs: 108 to 123.

In one embodiment, the present invention relates to an isolated polynucleotide comprising a nucleotide sequence encoding a light chain variable region comprising the amino acid sequence of any one of SEQ ID NOs: 92 to 107.

In one embodiment, the present invention relates to an isolated polynucleotide comprising a nucleotide sequence encoding a heavy chain variable region comprising the amino acid sequence of any one of SEQ NO: 131, SEQ NO: 133, SEQ NO: 135, SEQ NO: 137 or SEQ NO: 139.

In one embodiment, the present invention relates to an isolated polynucleotide comprising the nucleotide sequence of any one of SEQ NO: 130, SEQ NO: 132, SEQ NO: 134, SEQ NO: 136 or SEQ NO: 138.

In one embodiment, the present invention relates to an isolated polynucleotide comprising a nucleotide sequence encoding a light chain variable region comprising the amino acid sequence of any one of SEQ NO: 125, SEQ NO: 127 or SEQ NO: 129.

In one embodiment, the present invention relates to an isolated polynucleotide comprising the nucleotide sequence of any one of SEQ NO: 124, SEQ NO: 126 or SEQ NO: 128.

In one embodiment, the present invention relates to an isolated polynucleotide comprising a nucleotide sequence encoding a heavy chain comprising the amino acid sequence of any one of SEQ NO: 143, SEQ NO: 147, SEQ NO: 149, SEQ NO: 153 or SEQ NO: 155.

In one embodiment, the present invention relates to an isolated polynucleotide comprising the nucleotide sequence of any one of SEQ NO: 142, SEQ NO: 146, SEQ NO: 148, SEQ NO: 152 or SEQ NO: 154.

In one embodiment, the present invention relates to an isolated polynucleotide comprising a nucleotide sequence encoding a light chain comprising the amino acid sequence of any one of SEQ NO: 141, SEQ NO: 145 or SEQ NO: 151.

In one embodiment, the present invention relates to an isolated polynucleotide comprising the nucleotide sequence of any one of SEQ NO: 140, SEQ NO: 144 or SEQ NO: 150.

In one embodiment, the present invention relates to an isolated polynucleotide comprising a nucleotide sequence encoding a heavy chain comprising the amino acid sequence of any one of SEQ NO: 159, SEQ NO: 161 or SEQ NO: 163.

In one embodiment, the present invention relates to an isolated polynucleotide comprising the nucleotide sequence of any one of SEQ NO: 158, SEQ NO: 160 or SEQ NO: 162.

In one embodiment, the present invention relates to an isolated polynucleotide comprising a nucleotide sequence encoding a light chain comprising the amino acid sequence of SEQ NO: 157.

In one embodiment, the present invention relates to an isolated polynucleotide comprising the nucleotide sequence of SEQ NO: 156.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the anti-PD-1 antibody or the antigen-binding fragment thereof. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The invention is further described in the following examples, which are not intended to limit the scope of the invention.

### EXAMPLES

### Example 1: Antibody Generation (Immunization)

MHC Type A, C, D, E, H, G strain mice were immunized with recombinant monomeric human PD-1 or human PD-1-humanFc-His protein. The gene symbol for this recombinant protein is PDCD1 and the GenelD is 5133. Serology was then assessed by flow cytometry using CHO-human PD-1 cells, expressing human PD-1 antigen for binding. Selected serologically positive mice were given a final boost before B-cell isolation. All selected mice exhibited positive antibody titers in the sera. At positive serology, splenocytes were harvested for recovery of antigen-specific B-cells. All procedures were carried out in accordance with protocol approved by IACUC (Institutional Animal Care and Use Committee).

### Example 2: Production of Humanized Anti-PD-1 Antibodies

Mouse lead antibody 723C2 was converted to chimeric antibodies consisting of the mouse variable domain of 723C2 and a human constant IgG1WT, IgG1KO, or IgG4Pro domain. Sequences of mouse antibody 723C2 light chain variable region (VK) and heavy chain variable region (VH) are shown in Tables 1 and 2 herein above. The IgG4Pro has one replacement mutation (Ser228Pro) that prevents Fab-arm exchanging. The IgG1KO has two mutations in the hinge region, Leu234Ala and Leu235Ala, to reduce effector function (ADCC). Chimeric antibodies are generated to confirm the function of the antibody and to ensure the correct sequence has been obtained. Sequences of the chimeric 723C2 in human IgG1WT, IgG1KO, and IgG4Pro formats are shown in Table 8. The chimeric 723C2 in human IgG1WT and IgG4Pro contains a mutation in H-CDR3, DC to DY. However, the chimeric 723C2 in human IgG1KO does not have the mutation. The mutation in this site is highlighted in Table 8. The variable region of the antibody is then humanized through a design and screening process. A library was made where human and mouse residues were varied in such a way that in any given position there could either be a human or mouse residue. Such a library was made for those amino acids that were different between human germline and mouse antibody. Only the clones that retain the function of the parent mouse antibody were selected. Representative humanized variable regions for antibody 723C2 are shown in Tables 5 and 6.

In this manner, Antibody A, Antibody B, Antibody C, Antibody D, and Antibody E were humanized antibodies derived from mouse antibody 723C2 (cloned into a human IgG4Pro/kappa backbone). Antibodies A, B, C, D and E are shown in Table 7.

### Example 3: Binding of Antibodies to Recombinant PD-1 Protein

A) Kinetics and affinity of chimeric anti-PD-1 antibodies in human IgG4Pro backbone binding to recombinant human PD-1 are shown below (Table 9). Kinetics and binding affinities were measured using the ProteOn™ XPR36 (Biorad, Hercules, CA) surface plasmon resonance optical biosensor using material generated from transient transfection following single column purification.

TABLE 9

| Antibody | ka(1/Ms) | kd(1/s) | KD(nM) |
|---|---|---|---|
| Chimeric 306E6 | 1.36E+05 | 7.65E−03 | 56.4 |
| Chimeric 307A3 | 1.16E+05 | 7.40E−03 | 63.5 |
| Chimeric 313C12 | 6.71E+04 | 5.77E−05 | 0.859 |
| Chimeric 414A12 | 1.80E+05 | 2.69E−04 | 1.50 |
| Chimeric 502H1 | 5.47E+04 | 3.49E−04 | 6.39 |
| Chimeric 701C1 | 2.34E+04 | 3.05E−04 | 13.0 |
| Chimeric 701E9 | 8.83E+04 | 1.43E−04 | 1.62 |
| Chimeric 703D10 | 3.46E+04 | 3.29E−04 | 9.49 |
| Chimeric 708E4 | 4.31E+04 | 3.48E−04 | 8.08 |
| Chimeric 709A6 | 8.74E+04 | 4.83E−04 | 5.52 |
| Chimeric 718C2 | 2.84E+04 | 1.13E−04 | 3.98 |
| Chimeric 723C2 | 2.26E+05 | 5.82E−04 | 2.58 |
| Chimeric 803E6 | 1.06E+05 | 5.30E−04 | 5.02 |
| Chimeric 811G3 | 8.77E+04 | 8.01E−04 | 9.14 |
| Chimeric 814E10 | 8.07E+04 | 1.49E−04 | 1.85 |
| Chimeric 820C3 | 1.06E+04 | 2.86E−04 | 2.71 |

B) Affinities were measured for humanized anti-PD-1 antibodies derived from mouse antibody 723C2. Kinetic binding data, measured using the ProteOn™ XPR36 (Biorad, Hercules, CA) surface plasmon resonance optical biosensor and globally fit to a 1:1 binding model, demonstrated the interactions with recombinant human PD-1 to be in the range of 1 nM˜ 10 nM (Table 10). Antibody PD1AB-6-4P (antibody in IgG4Pro backbone disclosed in WO2017/058859 to Celgene) was also tested.

TABLE 10

| | ka (1/Ms) | kd (1/s) | KD (nM) |
|---|---|---|---|
| Chimeric723C2 | 2.26E+05 | 5.82E−04 | 2.58 |
| in IgG4Pro | | | |
| Antibody A | 1.59E+05 | 4.34E−04 | 2.73 |
| Antibody B | 2.59E+05 | 4.21E−04 | 1.62 |
| Antibody C | 1.75E+05 | 4.63E−04 | 2.65 |
| Antibody D | 2.22E+05 | 5.74E−04 | 2.58 |
| Antibody E | 1.78E+05 | 4.92E−04 | 2.77 |
| PD1AB-6-4P | 2.10E+05 | 4.60E−03 | 22.0 |

C) Affinity and kinetic data for the anti-PD-1 antibodies binding to cynomologous PD-1 were measured on the ProteOn™ XPR36 surface plasmon resonance optical biosensor, and globally fit to a 1:1 binding model (Table 11). Antibody, PD1AB-6-4P was also tested.

TABLE 11

| Antibody | ka (1/Ms) | kd (1/s) | KD (nM) |
|---|---|---|---|
| Chimeric 723C2 | 1.85E+05 | 7.21E−03 | 38.9 |
| in IgG4Pro | | | |
| Antibody A | 1.78E+05 | 4.73E−03 | 26.5 |
| Antibody B | 1.69E+05 | 5.58E−03 | 32.9 |
| Antibody C | 1.54E+05 | 6.33E−03 | 41.0 |
| Antibody D | 1.49E+05 | 6.74E−03 | 45.2 |
| Antibody E | 1.73eE+05 | 6.63E−03 | 38.2 |
| PD1AB-6-4P | NA | NA | 25.0 |

D) Molecular Selectivity to human PD-1

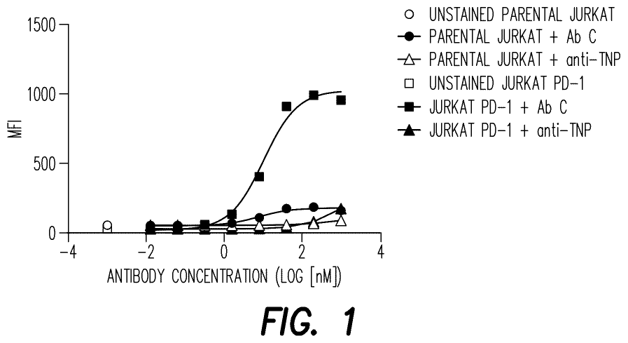

Selectivity of anti-PD-1 antibody to human PD-1 protein in a cell-based assay was evaluated by flow cytometry. Parental Jurkat cells that do not express the human PD-1 protein or Jurkat cells expressing the human PD-1 protein were incubated with the anti-PD-1 antibody labeled with AlexaFluor 647 at the concentrations indicated below. As a control, parental and PD-1 expressing Jurkat cells were incubated with anti-TNP isotype control antibodies. Following incubation, the cells were washed to remove non-bound antibody, fixed in PFA, and then washed in staining buffer. Binding of the antibody to the Jurkat cells was evaluated by flow cytometry. Unstained cells were also evaluated by flow cytometry as a negative control. The anti-PD-1 antibody selectively binds to human PD-1 up to at least 1 micromolar, as indicated by dose-dependent antibody binding to Jurkat cells that express the human PD-1 protein and the lack of binding of AlexaFluor 647 labeled anti-PD-1 antibody to parental Jurkat cells lacking PD-1 expression. The results of a representative experiment using Antibody C (Ab C) are shown in FIG. 1.

### Example 4: Competition Binding Assay of Human PD-1-Fc Binding to Human PD-L1-Fc

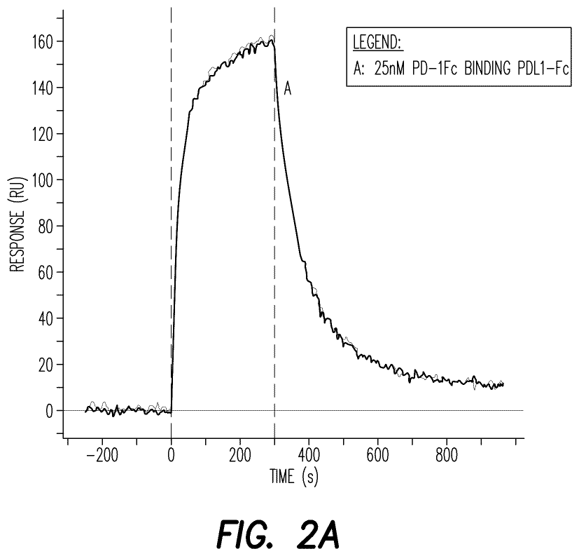

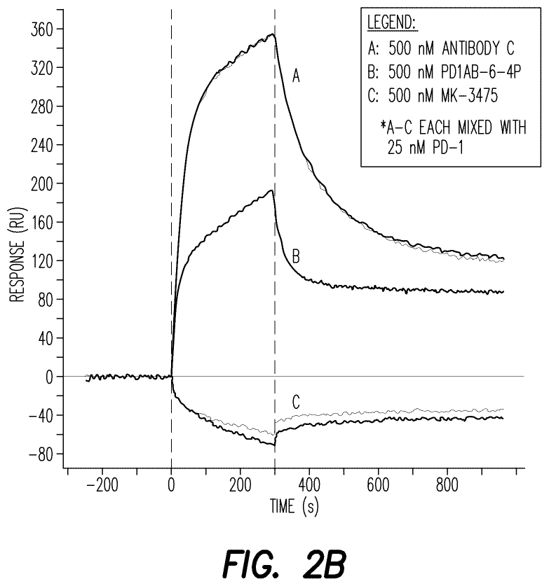

Human PD-L1-Fc was amine coupled onto channels 1-3 of a GLM chip on a BioRad ProteOn™ XPR36 surface plasmon resonance optical biosensor instrument at a concentration of 60 μg/mL; the three test antibodies, Antibody C, MK-3475 (pembrolizumab), and PD1AB-6-4P were amine coupled onto channels 4, 5, and 6, respectively at 30 μg/mL. Human PD1-Fc was injected across channels 1-6 on the chip surface at a concentration of 25 nM. The sensorgram indicates the specific binding between PD-L1 and the PD-1 receptor (FIG. 2A). 500 nM Antibody C, MK-3475, and PD1AB-6-4P were pre-mixed with 25 nM PD1-Fc and injected as analyte across all channels on the chip to assess whether the individual antibodies inhibit binding of PD-L1 to PD-1. Both Antibody C and PD1AB-6-4P are noncompetitive with PD-L1 for binding to the PD-1 antigen as demonstrated by the sensorgrams. MK-3475 and PD-L1 are potential binding blockers of one another with PD-1 based on the non-binding sensorgrams observed in the competition assay (FIG. 2B).

### Example 5. Enhanced Binding of PD-L1 to PD-1 in the Presence of Anti-PD-1 Agonist Antibody

PD-1/PD-L1 interactions were interrogated in the presence of the PD-1 agonist antibody 723C2 in human IgG4Pro backbone without the DC to DY mutation in H-CDR3. Multiple assays were utilized to demonstrate that antibody 723C2 enhanced the binding of PD-L1 to PD-1. A biochemical ELISA-based assay was utilized to evaluate the binding of PD-1 to platebound PD-L1 (BPS Bioscience). White 96-well microplates were coated with 50 μl of PD-L1 at 2 μg/ml in PBS overnight at 4° C. Supernatants were removed and plates were washed three times with 1× immunobuffer provided by the manufacturer BPS Bioscience (Cat #72005) followed by blocking with blocking buffer for one hour at room temperature (RT). Antibodies, along with the relevant controls, were added followed by addition of PD-1 biotin at 0.5 ng/ml (10 ng) for two hours at room temperature. Plates were blocked with blocking buffer for 10 minutes. A streptavidin horseradish peroxidase secondary antibody was added to the washed plates for an hour followed by washing with PD-1 assay buffer. Plates were blocked for 10 minutes. Chemilumiscent substrate mixture was added to the plate just before reading. The chemiluminescent signal was read on a luminometer (Envision) or on a microtiter plate capable of reading chemiluminescence.

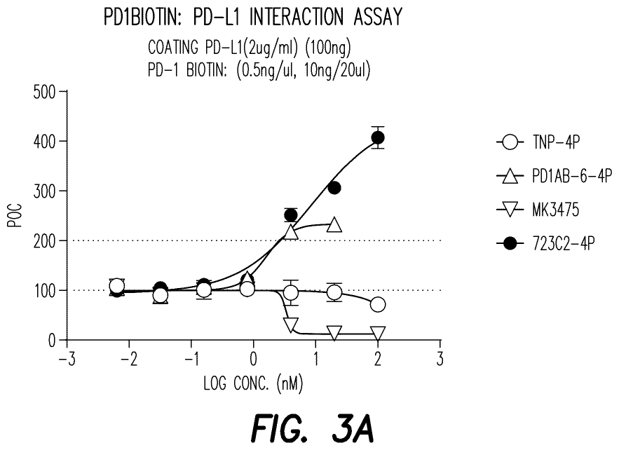

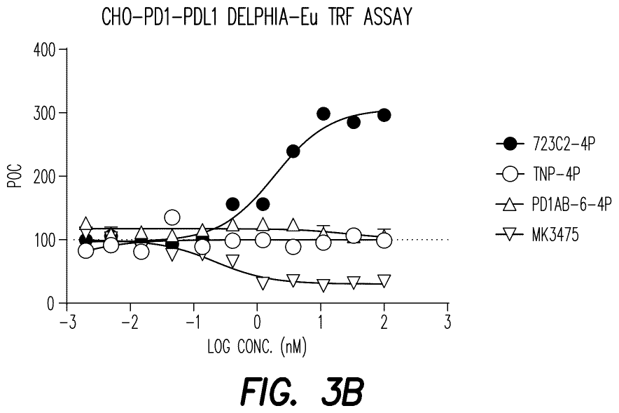

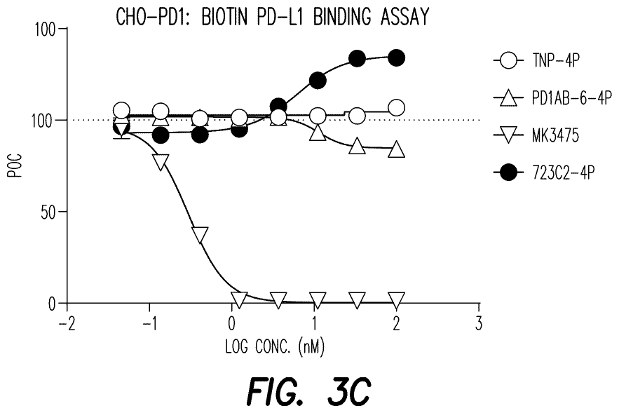

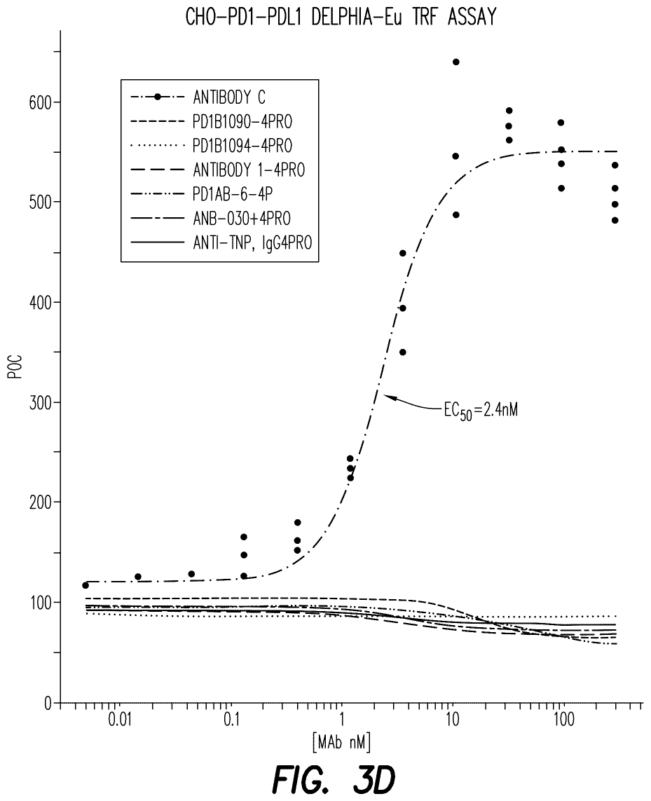

Enhanced interactions of PD-1 with PD-L1 was observed in the presence of antibody 723C2, as indicated by increased chemiluminescence signal compared to isotype control treated samples (FIG. 3A). Antibody 723C2 is designated as 723C2-4P in FIG. 3A. This is in contrast to MK3475, a known anti-PD-1 antagonist antibody, which blocked the PD-L1-PD-1 interaction. Antibody PD1AB-6-4P demonstrated limited enhancement of PD-1-PD-L1 in this assay (FIG. 3A).

A cell-based assay was utilized to confirm the ELISA-based results demonstrating the enhancement of PD-1/PD-L1 interactions in the presence of the antibody 723C2. Here, the interaction of PD-1/PD-L1 was evaluated by measuring the binding of soluble PD-1 to CHO cells over-expressing PD-1 with a DELFIA (dissociation-enhanced lanthanide fluorescence immunoassay) receptor-ligand binding assay (Perkin Elmer).

10,000 cells were plated and incubated overnight at 37° C.±5% CO2 incubator (humidified incubator). Biotin labeled PD-L1 EC 10 (130 nM) and 10 μl of PD-1 antibody was added to each well and incubated at RT for 1 hour. Plates were washed twice with 50 μl of 1× TRF Wash Buffer. 20 μL of Eu-Streptavidin reagent was added to the assay plate, and incubated at room temperature for one hour. Enhancement Solution was added and incubated for 30 minutes at RT. Plates were read on a Fluorescence Plate Reader (Excitation: 320 or 340 nm, Emission: 615 nm). In this assay, and in confirmation of the ELISA assay, the presence of antibody 723C2 in this cell-based assay enhanced the PD-1/PD-L1 interactions (FIG. 3B). Antibody 723C2 is designated as 723C2-4P in FIG. 3B.

A second cell-based assay where PD-1 was expressed on CHO cells was also utilized to evaluate PD-1/PD-L1 interactions. In this assay, PD-L1-multimer binding to CHO cells expressing PD-1 was measured by flow cytometry. 50 μl of 2×10⁶ cells/ml were added to each well (100,000 cells/well). Cells were centrifuged and re-suspended in 50 μl of the indicated antibody concentrations and incubated for 60 minutes on ice. PDL1-biotin and Streptavidin-APC were combined in stain buffer (1 μg/ml PDL1-biotin+0.25 μg/ml Streptavidin-APC). Add 50 μl of 2×PDL1-biotin/Streptavidin-APC mixture to the cells and incubate on ice for 60 minutes. Cells were washed and resuspended in 180 μl stain buffer +20μ PFA and data was acquired on a BD LSR II. As indicated in FIG. 3C, this cell-based assay also demonstrated the enhanced binding of PD-L1 to PD-1 in the presence of antibody 723C2. Antibody PD1AB-6-4P had no effect on PD-L1 binding, while the MK3475 antagonist antibody inhibited binding of PD-L1 to PD-1 (FIG. 3C). Antibody 723C2 is designated as 723C2-4P in FIG. 3C.

A CHO PD-1-PD-L1 Delphia-Eu TRF assay as described above was also performed with Antibody C, antibody PD1AB-6-4P, Antibody 1-4Pro, antibody PD1B1090-4Pro, antibody PD1B1094-4Pro and antibody ANB-030-4Pro. Antibody 1-4Pro comprises the heavy chain and light chain variable regions of Antibody 1 described in WO2019/168745 to Eli Lilly, in a IgG4-Pro backbone. Antibody PD1B1090-4Pro and antibody PD1B1094-4Pro comprise the heavy chain and light chain variable regions of PD1B1090 and PD1B1094, respectively, described in WO2018/226580 to Janssen Biotech, in a IgG4-Pro backbone. Antibody ANB-030-4Pro comprises the heavy chain and light chain variable regions of antibody ANB-030, described under CAS number CAS 2412764-40-8 in a IgG4-Pro backbone (also corresponds to the heavy chain and light chain variable regions of APE12537, described in WO2020/247648 to Anaptysbio). An anti-TNP antibody in IgG4-Pro backbone was also included.

Antibody C showed consistent (N=3) enhancement of PD-1\PDL-1 binding in a concentration dependent manner (FIG. 3D). All other anti-PD-1 agonists consistently showed no enhancement of PD-1\PDL-1 binding (FIG. 3D).

### Example 6: Functional Cell Assays, inhibition of NFAT activation in a THP-1/Jurkat-PD-1 agonist reporter assay

A THP-1/Jurkat PD1 NFAT co-culture assay was developed to assess the agonist activity of anti-PD1 antibodies generated from multiple campaigns. The THP-1 cell line was obtained from ATCC. The Jurkat reporter cell line was generated internally. The Jurkat reporter cells overexpress human PD-1 (hPD-1) on the cell surface and also express an NFAT-driven luciferase reporter to measure the activation status of the cells in response to stimulation. The Jurkat PD1 NFAT cells are activated with a CD3×CD33 BiTE in the presence of THP-1 cells. The anti-CD33 arm of the BiTE binds CD33 expressed on the THP-1 cells while the anti-CD3 arm binds the CD3 molecule on the Jurkat cells. The BiTE serves to engage the THP-1 and Jurkat cells resulting in the formation of an immune synapse between the two cells while activating the Jurkat cells. Activation of the Jurkat PD-1 NFAT cells is measured through the NFAT-driven luciferase reporter. This assay was run in the presence of anti-PD1 antibodies to identify agonist antibodies. Molecules that exhibited 20% or greater reduction in activation, as indicated by a loss of luciferase signal, were classified as agonist antibodies (Table 12). Anti-PD-1 antibodies 306E6 to 820C3 in Table 12 were on a mouse IgG1 backbone. Several of these antibodies were selected for additional profiling on a human IgG4Pro backbone (indicated as chimeric antibodies in Table 12).

TABLE 12

| | IC₅₀ Values (nM), | Emax Values (%), |
|---|---|---|
| | inhibition of NFAT | inhibition of NFAT |
| Antibody | activity | activity |
| 306E6 | 1.14 | 45.5 |
| 307A3 | 1.03 | 42.6 |
| 313C12 | 0.37 | 42.2 |
| 414Al2 | 0.39 | 45.0 |
| 502H1 | 0.34 | 48.9 |
| 701C1 | 1.69 | 53.2 |
| 701E9 | 0.22 | 46.0 |
| 703D10 | 10.9 | 47.4 |
| 708E4 | 0.17 | 48.7 |
| 709A6 | 0.04 | 29.8 |
| 718C2 | 0.82 | 50.8 |
| 723C2 | 0.14 | 43.7 |
| 803E6 | 0.38 | 48.3 |
| 811G3 | 0.14 | 44.5 |
| 814E10 | 0.25 | 40.7 |
| 820C3 | 0.14 | 48.7 |
| Chimeric 718C2 | 0.38 | 44.4 |
| Chimeric 703D10 | 0.39 | 41.9 |
| Chimeric 723C2 | 0.13 | 42.6 |
| Chimeric 820C3 | 0.14 | 40.0 |

### Example 7: Functional Cell Assays-Inhibition of IFNγ Production from Human PD-1 Knockin Splenocytes

The primary cell assay used to select the top anti-PD1 antibodies was the hPD1 knock-in mouse splenocyte assay. Spleens were collected from C57BL/6 mice that express human PD1 in place of mouse PD1. Splenocytes were isolated from the spleens and activated with anti-CD3 (clone 2C11) at a concentration of 0.1 μg/ml. T cell activation was measured after 48 hours by quantitating mIFNγ levels by MSD analysis (Meso Scale Discovery). The assay was run in the presence of anti-PD1 antibodies selected from the THP-1/Jurkat PD1 NFAT screening assay. The top molecules identified from this assay were selected based on % inhibition of mIFNγ (50% or greater) and sequence clade. Inhibition and IC50 values are shown in Table 13.

TABLE 13

| | IC₅₀ Values (pM), | Emax Values (%), |
|---|---|---|
| | Average, inhibition of | Average, inhibition of |
| Antibody | IFNγ | IFNγ |
| 306E6 | 22 | 19.5 |
| 703D10 | 3205 | 65 |
| 313C12 | 459 | 79 |
| 718C2 | 995 | 64.5 |
| 814E10 | 74.2 | 63 |
| 708E4 | 915 | 73.5 |
| 723C2 | 121.5 | 74 |
| 820C3 | 205 | 82 |

### Example 8: Functional Cell Assays, Inhibition of IFNγ Production from Human PBMC Assay

The anti-PD-1 agonist antibodies were further characterized for their ability to modulate T cell functional activity, as measured by IFNγ production, in a human primary cell assay. PBMCs were isolated from human whole blood and activated with1.5 pM of anti-CD3 (clone OKT3, BioLegend). T cell activation and function was assessed after 72 hours by quantitating hIFNγ levels by MSD analysis. Identified anti-PD-1 agonist antibodies were able to reduce IFNγ secretion compared to isotype control treated cells (Table 14A).

TABLE 14A

| | IC₅₀ Values (pM), | Emax Values (%), |
|---|---|---|
| | Geomean, | Average, |
| Antibody | inhibition of IFNγ | inhibition of IFNγ |
| chimeric 723C2 | 18.0 | 66.5 |
| in IgG4Pro | | |
| chimeric 820C3 | 38.5 | 56.7 |
| in IgG4Pro | | |
| Antibody A | 7.4 | 49.0 |
| Antibody B | 18.4 | 48.9 |
| Antibody C | 17.7 | 46.8 |
| Antibody D | 18.0 | 50.1 |
| Antibody E | 8.1 | 47.8 |
| PD1AB-6-4P | 31.5 | 40.1 |

Antibody C, Antibody 1-4Pro, antibody PD1B1090-4Pro, antibody PD1B1094-4Pro, antibody ANB-030-4Pro and abatacept were also tested in this assay. The results are shown in Table 14B. Antibody C, the variable regions of Antibody 1-4Pro, antibody PD1B1090-4Pro, antibody PD1B1094-4Pro and antibody ANB-030-4Pro in a IgG1 wild-type backbone and in a IgG1 KO backbone, and abatacept were also tested in this assay. The results are shown in Table 14C summarized below. In each experiment, five donors were tested.

TABLE 14B

| | IC₅₀ Values (nM), | |
|---|---|---|
| | Geomean, | Emax Values (%), |
| | inhibition of | Average, |
| Antibody | IFNγ | inhibition of IFNγ |
| Antibody C | 0.03 | 59 |
| Antibody 1-4Pro | 0.01 | 55 |
| PD1B1090-4Pro | 0.79 | 72 |
| PD1B1094-4Pro | 0.46 | 64 |
| ANB-030-4Pro | 0.27 | 66 |
| Abatacept | 0.39 | 66 |

TABLE 14C

| | IC₅₀ Values (nM), | Emax Values (%), |
|---|---|---|
| | Geomean, | Average, |
| Antibody | inhibition of IFNγ | inhibition of IFNγ |
| Antibody C | 0.03 | 59 |
| Antibody 1-IgG1WT | 0.01 | 46 |
| PD1B1090-IgG1WT | 0.07 | 83 |
| PD1B1094-IgG1WT | 0.12 | 79 |
| ANB-030-IgG1WT | 0.03 | 78 |
| abatacept | 0.39 | 66 |

The inhibition of IFNγ for the variable regions of Antibody 1-4Pro, antibody PD1B1090-4Pro, antibody PD1B1094-4Pro and antibody ANB-030-4Pro in a IgG1 KO backbone was less than 40% with IC₅₀ values above 30 nM.

### Example 9: Functional Cell Assays, Inhibition of IL-17A Production from Th17-Monocyte Co-Culture Assay

Anti-PD-1 agonist antibodies were tested for functional inhibition of IL-17 secretion by Th17 differentiated T cells. A primary cell co-culture assay was developed to assess modulation of IL-17 by PD-1. Human primary T cells isolated from PBMCs were Th17-differentiated under the following skewing conditions: CD4 T cells were stimulated with 0.5 μg/ml plate-bound anti-CD3 (clone UCHT1) for 4 days in Th17 skewing media (X-VIVO15 media+IL-13(10 ng/ml), IL-23(10 ng/ml), IL-6(10 ng/ml), IL-2(2 ng/ml), TGFβ (0.5 ng/ml), 5 μg/mL anti-IL4, 5 μg/mL anti-IFNγ). After 4 days, the cells were removed from the anti-CD3 coated plates and transferred to flasks containing Th17 skewing media. Following differentiation, Th17 cells were rested at least 3 days, then co-cultured with the autologous monocytes and re-stimulated with 40 fM anti-CD3 (clone OKT3) in the presence of the PD-1 antibodies. The co-culture system was required due to the Fc requirements necessary for the anti-PD-1 antibodies to demonstrate agonist activity. Inhibition of IL-17 was observed in this assay in the presence of anti-PD-1 agonist antibodies. Antibody IC₅₀ and maximum inhibition of the IL-17 response are shown in the table below (Table 15). Maximum inhibition was compared relative to an isotype control antibody.

TABLE 15

| | IC₅₀ Values (pM), | Emax Values (%), |
|---|---|---|
| | Geomean, | Average, |
| Antibody | inhibition of IL-17A | inhibition of IL-17A |
| Chimeric 723C2 | 14.4 | 70.0 |
| in IgG4Pro | | |
| Antibody A | 41.1 | 69.8 |
| Antibody B | 25.3 | 63.4 |
| Antibody C | 32.5 | 62.9 |
| Antibody D | 31.9 | 63.9 |
| Antibody E | 8.1 | 59.2 |
| PD1AB-6-4P | 175.8 | 67.5 |

### Example 10: Functional Cell Assays, Inhibition of IL-21 Production from Tfh-Monocyte Co-Culture Assay

An assay was developed to assess the ability of anti-PD-1 agonist antibodies to inhibit T follicular helper (Tfh) cell activity in vitro. CD4 T cells and autologous monocytes were obtained from ALLCELLS. T cells were skewed to the Tfh lineage by activating the cells with Dynabeads Human T-Activator CD3/CD28 (Gibco) in the presence of IL-23(25 ng/ml) and TGFβ (5 ng/ml) for 5 days and then combined with autologous monocytes in the presence of 4.5 pM anti-CD3 (clone OKT3, BioLegend) and anti-PD-1 agonist antibodies after cells were washed and activation beads removed. 24 hours later, supernatants were collected and assayed for the presence of IL-21 (Meso Scale Discovery, MSD V-Plex Human IL-21 Kit). IL-21 production of restimulated Tfh-differentiated cells was inhibited by anti-PD-1 agonist antibodies. Representative IC50 and Emax inhibition values are shown in Table 16.

TABLE 16

| | IC₅₀ Values (pM), | Emax Values (%), |
|---|---|---|
| | Geomean, | Average, |
| Antibody | inhibition of IL-21 | inhibition of IL-21 |
| Chimeric 723C2 | 12 | 85 |
| in IgG4Pro | | |
| Antibody C | 6 | 84 |
| PD1AB-6-4P | 50 | 71 |

### Example 11: Role of FcgR Interactions on PD-1 Agonist Activity

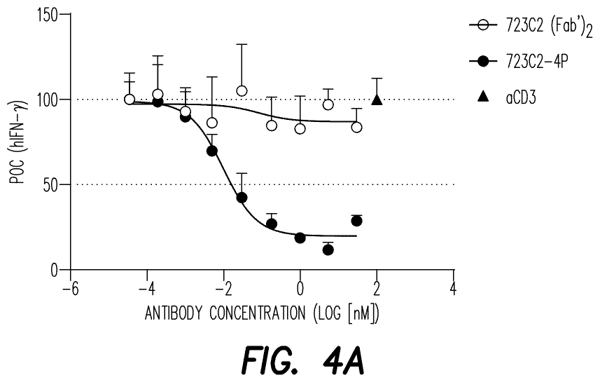

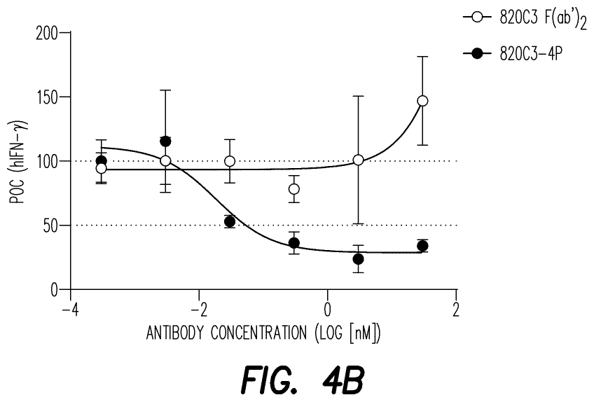

The role of Fc-Fcg receptor interactions on functional activity of the agonist antibodies was characterized by utilizing candidate anti PD-1 agonist antibodies on different backbone formats (IgG1 wild type, IgG1 KO, or IgG4 Pro) or divalent antibody fragments (F(ab′)2 fragments). The functional activity of the antibody variants was evaluated by the ability to modulate IFNγ production from activated T cells in the human PBMC assay described above. Functional agonist activity, as measured by a reduction in IFNg production, is lost with the divalent F(ab′)2 fragments of the parental 723C2 and 820C3 antibodies (FIGS. 4A and 4B). In contrast, the full-length antibody in a human IgG4Pro backbone inhibited IFNγ production in dose-dependent manner (FIGS. 4A and 4B, designated as 723C2-4P and 820C3-4P, respectively). In these assays, human PBMCs were isolated from whole blood and activated with 1.5 pM of anti-CD3 clone OKT3 in the presence of the anti-PD-1 antibody or the F(ab′) 2 fragment of the indicated anti-PD-1 antibody. After 72 hours, human IFN gamma cytokine levels in the supernatant were measured by MSD analysis.

As this suggested that Fc interactions are required for functional agonist activity of the anti-PD-1 antibody, the 723C2 antibody was generated on IgG1WT, IgG1KO, and IgG4Pro backbones to further characterize these interactions (723-IgG1WT, 723-IgG1KO and 723-IgG4Pro, respectively, in Table 17 below). Both IgG1 WT and IgG4 Pro bind to human Fc receptors to differing degrees, while the IgG1 KO backbone has greatly reduced binding to Fc receptors. The anti-PD-1 agonist antibody on the IgG4 Pro demonstrated the highest degree of inhibition of IFNγ in the human PBMC assay, while the antibody on the IgG1 KO demonstrated greatly reduced activity (Table 17). Collectively, these data indicate that functional agonism of the anti-PD-1 antibody is dependent on Fc interactions.

TABLE 17

| | % Inhibition | IC 50 |
|---|---|---|
| | IFNγ (mean, SD) | (geomean (nM), SD) |
| 723-IgG4Pro | 57 ± 17 | 0.03 ± 0.136 |
| 723-IgG1WT | 45 ± 16 | 0.015 ± 0.021 |
| 723-IgG1KO | 29 ± 17 | 0.02 ± 0.4 |

### Example 12: In Vivo Model—Xenogeneic CD4⁺ T Cell GvHD Model

An in vivo xenogeneic CD4⁺ T cell GvHD mouse model was used to test the efficacy of the PD-1 agonist antibodies. Eight NSG mice per group (NOD. Cg-Prkdo^{scid} ∥2rg^{tm1Wjl}/SzJ, The Jackson Laboratory) were injected IV with 5×10⁶ CD4⁺ T cells (purified by negative selection) from healthy donor leukopaks. Mice were dosed two times per week at 0.625 mg/kg IP according to the following; Group 1:723 (IgG4-Pro), Group 2: PD1AB-6-4P (IgG4-Pro), Group 3: anti-TNP isotype (IgG4-Pro), Group 4: Avelumab (hIgG1-LALAPG), Group 5: anti-TNP isotype (hIgG1-LALAPG), Group 6: CTLA4-Ig (hIgG1-LALA). TNP is trinitrophenol. LALA represents the Leu234Ala/Leu235Ala mutations commonly used for disrupting antibody effector function. PG represents the Pro329Gly mutation, which eliminates effector function by preventing binding to Fc gamma receptors. Three experimental repeats were run, each with a unique donor. By week four, significant inhibition of human cell accumulation was noted in Groups 1, 2 and 6 compared to their isotype-matched controls for all donors tested (Table 18). Quantification of inflammatory cytokines at week four showed a significant reduction in the levels of human IFNγ, TNFα, and IL-10 in all donors (Table 19). Human IL-1B, IL-2, IL-4, IL-6, IL-8, IL-12p70, and IL-13 were also tested, but all were below the limit of detection for the assay.

TABLE 18
HUMAN CD45⁺ CELL ACCUMULATION

| | % hCD45⁺ cells (mean ± SD) | | | | | |
|---|---|---|---|---|---|---|
| | Donor 1 | | Donor 2 | | Donor 3 | |
| Group 1 | 2.23 | (2.83) | 3.52 | (6.37) | 0.63 | (0.68) |
| Group 2 | 14.86 | (13.43) | 3.63 | (3.12) | 1.31 | (0.92) |
| Group 3 | 43.57 | (18.33) | 21.92 | (14.73) | 15.07 | (14.48) |
| Group 4 | 40.70 | (19.29) | 21.09 | (9.41) | 24.70 | (16.73) |
| Group 5 | 48.98 | (17.87) | 25.22 | (14.73) | 19.86 | (3.95) |
| Group 6 | 3.24 | (1.88) | 6.63 | (5.50) | 1.76 | (1.96) |

TABLE 19
HUMAN PLASMA CYTOKINE PRODUCTION

| | hIFNγ (pg/mL, mean ± SD) | | | hTNFα (pg/mL, mean ± SD) | | | hIL-10 (pg/mL, mean ± SD) | | |
|---|---|---|---|---|---|---|---|---|---|
| | Donor | Donor | Donor | Donor | Donor | Donor | Donor | Donor | Donor |
| | 1 | 2 | 3 | 1 | 2 | 3 | 1 | 2 | 3 |
| Group | 331 | 6616 | 372 | 1 (1) | 8 (17) | 1 (1) | 1 (1) | 2 (5) | 0 (0) |
| 1 | (333) | (16780) | (742) | | | | | | |
| Group | 11475 | 2846 | 631 | 9 (10) | 6 (4) | 1 (1) | 6 (8) | 1 (1) | 0 (0) |
| 2 | (18275) | (3079) | (606) | | | | | | |
| Group | 40323 | 24450 | 11967 | 18 (7) | 43 (27) | 19 (13) | 12 (6) | 12 (6) | 4 (2) |
| 3 | (36540) | (15273) | (9048) | | | | | | |
| Group | 76077 | 28643 | 13302 | 29 (15) | 37 (15) | 19 (14) | 18 (7) | 10 (3) | 4 (3) |
| 4 | (61582) | (12275) | (11700) | | | | | | |
| Group | 39655 | 20180 | 15435 | 21 (9) | 34 (14) | 20 (9) | 16 (8) | 11 (7) | 4 (3) |
| 5 | (48118) | (9671) | (10619) | | | | | | |
| Group | 837 | 7163 | 871 | 1 (1) | 13 (15) | 3 (4) | 1 (1) | 8 (15) | 1 (1) |
| 6 | (1498) | (9580) | (1907) | | | | | | |

### Example 13: Pharmacokinetic Studies in Cynomolgus Monkey

Pharmacokinetics (PK) of Antibody C were assessed in male cynomolgus monkeys of Chinese origin following a single intravenous (IV) bolus dose of 0.1, 0.3 and 1.5 mg/kg or subcutaneous (SC) dose of 1.5 mg/kg (n=3 per group). Serum concentrations of Antibody C were determined using two different MSD immunoassay formats: (1) A “total” drug generic anti-human capture and detection assay and (2) A “free” drug assay with antigen (PD1-ECD) capture and anti-human detection. The PK profiles of both assays were superimposable, suggesting that endogenous sPD-1 did not interfere with the measurement of Antibody C and had little to no effect on TMDD. Antibody C demonstrated dose dependent CL (using both free and “total” assays), between 0.1 and 0.3 mg/kg suggestive of target mediated drug disposition (TMDD) contributions to overall clearance. The summary of NCA pharmacokinetic parameters for each of the respective doses is shown in Table 20 below.

TABLE 20

| Dose | | CL | | AUC | T_{1/2} | MRT | |
|---|---|---|---|---|---|---|---|
| (mg/kg) | Route | (ml/d/kg) | Vc(ml/kg) | (μg · h/ml) | (days) | (days) | F % |
| 0.1 | i.v. | 14.3 | 35.6 | 171 | 2.9 | 4.1 | — |
| 0.3 | i.v. | 6.2 | 38.6 | 1,190 | 8.7 | 12.1 | — |
| 1.5 | i.v. | 3.0 | 33.9 | 12,400 | 18 | 25 | — |
| 1.5 | s.c. | — | — | 12,000 | — | 22.7 | 96.9 |

### Example 14: Transfection and production in CHO cells and biophysical data

Transfection and production in CHO cells:

CHO-E cells are transfected at ˜2×10E6 cells/mL in Irvine BalanCD Transfectory CHO+4 mM L-glutamine (or Glutamax). Amounts required for 1L transfection are 0.15 mg of HC DNA plus 0.3 mg of LC DNA and 1.05 mg Filler DNA (Herring sperm) and 0.15 mg XBP1 DNA. The DNA is diluted in 100 mL of OptiPro SFM and sterile filtered through a 0.2 μm filter. 0.75 ml of Mirus TransIT Pro transfection reagent is added to the diluted DNA mixture and the DNA complex immediately added to the prepared CHO-E cells, and the shake flask returned to the 37° C., 5% CO2 shaker at 140 rpm. 24 hours post-transfection, temperature is shifted to 32° C., 2 mL of Gibco Anti-Clumping Agent and 100 ml Irvine Transfectory Supplement is added to the transfected cells. Five days post-transfection shaker temperature is shifted to 30° C. 200 mL of Irvine Transfectory Supplement is added between day 5 or day 7, depending on when glucose levels drops between 2 g/L−1 g/L. The transfected cultures is maintained for 10 days. Harvest is done by spinning down the cells, followed by sterile filtration through a 0.2 μm PES filter (Thermo Scientific). After harvest, the clarified cell culture supernatants were sampled for titer by ForteBio/Pall Octet Red 96 instrument with Protein A biosensors as follows.

The titers for Antibody A, Antibody C, and Antibody E were between 18-38 mg/L, with about 80% recovery from protein purification, and more than 98% monomer after SEC purification. Proteins were buffer-exchanged in a final buffer containing 10 mM histidine-HCl, pH 6.0 and are stable at 4° C. for at least 4 months and with solubility up to 180 mg/ml in this buffer.

TABLE 21

| | Protein A Column | | | IEX Column | |
|---|---|---|---|---|---|
| | Titer | Yield | | Yield | |
| | (mg/L) | (mg/L) | Recovery | (mg/L) | Recovery |
| Antibody A | 22/18 | 25/21 | 114%/117% | 18/18 | 71%/83% |
| Antibody C | 23 | 22 | 95% | 18 | 83% |
| Antibody E | 38 | 38 | 100% | 31 | 80% |

TABLE 22

| | Quality | Stability | | Solubility |
|---|---|---|---|---|
| | SEC fresh | SEC 1 week at | SEC 4 weeks at | AUC at 180 |
| | (% M) | 40° C. (% M) | 40° C. (% M) | mg/ml (% M) |
| Antibody A | 99.08 | 98.41 | 97.55 | 97.7 |
| Antibody C | 98.99 | 97.87 | 96.68 | 95.7 |
| Antibody E | 98.61 | 97.80 | 96.96 | 99.9 |

AUC: Analytical Ultracentrifugation as measured by the sedimentation velocity method at concentrations of 0.5-1 mg/ml;
SEC: Size exclusion chromatography;
% M: percent monomer.

### Example 15: Bi-Specific Antibodies

### Materials and Methods

Mouse antibodies and Reagents. anti-hPD1 (EH12.2H7) (Biolegend, 329912); Anti-hCD48 (Bio-gems, 10511-25-500); IgG1 (cat #16-4714-85), anti-hCD3 (OKT3) (16-0037-85), anti-hCD3 (UCHT1) (16-0038-85) and anti-CD11a (140011982) from eBiosciences; anti-hCD71 (Southern Biotech, 9670-14). aCD3/aCD28 human T-cell activator Dynabeads (Gibco, 11131D)

Imagestream. Jurkat PD-1 cells were incubated in XVIVO 15 medium (Lonza) on ice for 10 minutes with AF-488 Cholera Toxin (Life Technologies, V-34403) and crosslinking antibody (Jackson ImmunoResearch) and either APC aCD3 (Biolegend, 317318), PV786 aPD-1 (Biolegend, 329930) or APC aCD48 (Sigma, SAB4700193). Cells were activated by transfer to pre-warmed X-VIVO 15 and allowed to incubate for an additional 12 minutes. Cell activation was stopped by addition of cold PBS-2% PFA (approximately 1:10 ratio, cells: PFA), and cells were incubated in fix solution for 20 minutes on ice. Cells were washed and resuspended in XVIVO and analyzed for cap formation and perimeter threshold using the Imagestream software.

Flow cytometry. 1×10⁵ Jurkat, Jurkat-PD-1 or aCD3/aCD28-stimulated primary human T cells were incubated for 1 hr at 4° C. with 1 mg/ml of primary MAbs, or where bispecific molecules were tested, 8-point binding curves were generated from a starting concentration of 6.25 mg/ml and serially diluted 1:4. Cells were washed and stained for 1 hr at 4° C. with a 1:100 dilution of PE-anti-mouse Ig (Life Technologies, P852), or 1:800 dilution of PE-Goat anti-human F(ab′)₂ (Invitrogen AHI1707), respectively. Samples were washed, fixed in 1× fix/lyse buffer (eBioscience, 00-5333-57) and analyzed on an LSR2 (BD)

PD-1 complementation assay. 2×10⁴ Jurkat T cells overexpressing full-length PD-1-PK and intracellular full-length SHP1-EA fusion proteins were purchased from DiscoverX (DRX-BI-080515A) and cultured following manufacturer description. Cells were resuspended in cell plating media (DiscoverX, 93-0563R4B), and pre-incubated with primary mouse or human antibodies at 4° C. for 30 min. Depending of experiment, cells were additionally pre-incubated with 10 mM of pan-Src kinase inhibitor PP2 (Abcam, ab120308), or the inactive analog PP3 (Abcam, ab120617). Cells were washed and treated with or without crosslinking secondary goat anti-mouse IgG (Thermo Scientific, 31170). Cells were transferred to 384-white Opti-Plates (PerkinElmer), received Flash Detection reagent (DiscoverX, 93-0247), and read in an EnVision Plate Reader (Perkin Elmer).

Primary huT cell activation. Primary human Pan-T cells (AllCells, PB009-1F) were labeled with 500 nM of Cell Trace Violet (Life technologies, cat #c34557). Epoxy-dynabeads M450 (Invitrogen, 14011) were covalently coated with 2.5 mg of mouse Abs/107 beads following manufacturer instructions. Cells were left unstimulated or were stimulated with plate bound anti-CD3 (UCHT1) (250 and 500 ng/ml) in the presence of Ab coated epoxy beads. Cells were harvested after 96h, stained with BV510 anti-CD4 (BD, 562970) and PeCy7 anti-CD8 (BD, 335787) Abs, and cell proliferation was analyzed by Cell Trace Violet dilution in LSR2 (BD). Primary memory CD4⁺/CD45RO⁺ T cells (AllCells, PB009-7F) were stimulated with 1 mg/well of plate bound anti-CD3 (UCHT1) in presence of 1 mg/well of plate bound isotype control (ISO) or BsAbs, respectively. Culture supernants were harvested at 72 h and analyzed for IL-2 and IL-10 secretion (MSD).

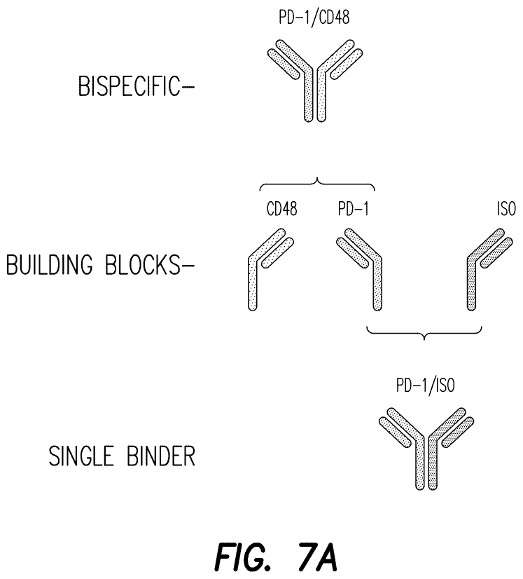

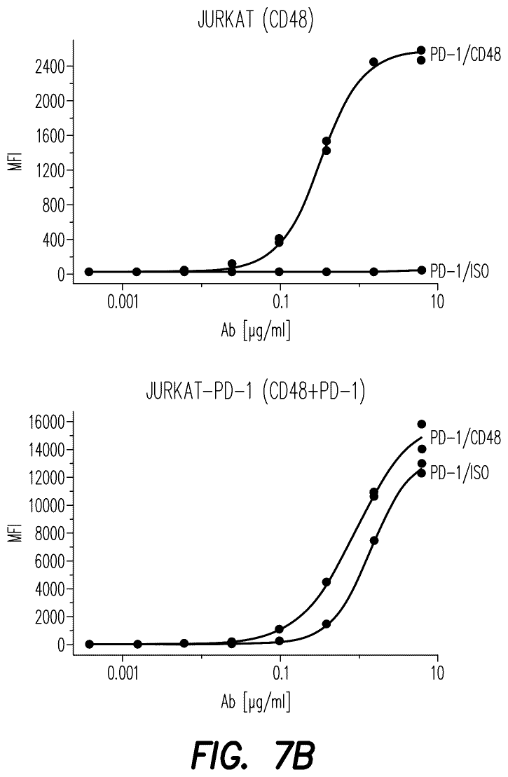

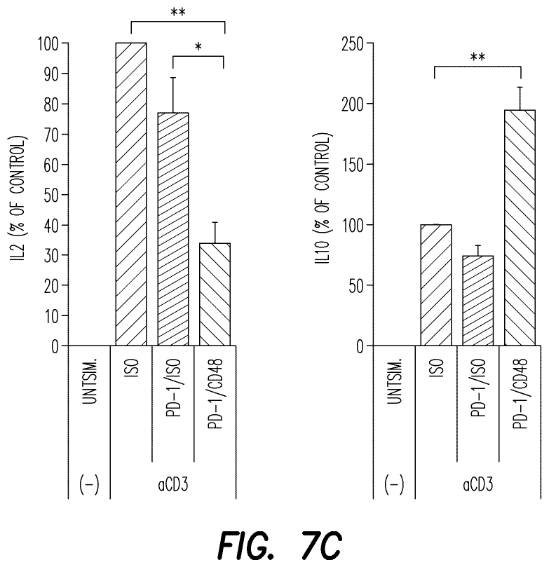

BsAbs generation and construct design. Bi-specific antibodies (BsAbs) were generated from published anti-CD48 (US2012/0076790) and anti-PD1 (WO 2011/110621A1) sequences that were used as building blocks. Bispecific constructs were designed with knob-into-hole technology to facilitate heterodimerization of two different target variable regions (IgG1-KO) to generate BsAbs containing anti-PD-1 and anti-CD48 (PD-1/CD48), or anti-PD-1 and anti-TNP as control (PD-1/ISO) (FIG. 7A). Variable-region sequences obtained for respective targets were cloned into pTT-5 (licensed from National Research Council Canada) expression vectors containing human constant regions. Briefly, variable region amino acid sequences were codon-optimized for mammalian expression. Light and heavy chains of the target V-gene were cloned into the same expression vector containing a joining linker segment. The vector was linearized by restriction-enzyme digest using EcoRI and Nhel recognition sites. DNA sequences for variable regions were ordered as G-blocks (dsDNA) from Integrated DNA Technologies (IDTDNA) with overlapping homologous ends to the vector and adjoining linker segment. G-blocks were then joined via Gibson assembly method (NEBuilder HiFi kit, New England Biolabs, cat #E5510S) according to the manufacturer's protocol. Traditional cloning was then completed by transforming the assembly mixture into competent cells (NEB 5-alpha C2987, New England Biolabs) and then grown overnight at 37C on LB agar plates with 100 μg/ml carbenicillin (Teknova). Individual colonies were picked and grown at 37C overnight in LB media with carbenicillin. Positive clones for insert were confirmed by sequence analysis using Lasergene software package (DNAstar). Sequence-confirmed plasmid DNA was scaled-up in 0.5L cultures and then purified via Plasmid Plus megaprep kit (Qiagen, cat #12981) according to the manufacturer's protocol.

CHO-E Transient Transfection. CHO-E cells are transfected at 2e6 cells/mL in FS-CHO supplemented with 2 mM Glutamine. For a 1L mAb transfection volume, 1 mg light chain (LC) plasmid DNA and 0.5 mg heavy chain (HC) plasmid DNA are diluted in 100 mL of OptiPro SFM (Gibco) and sterile filtered through a 0.2 μm filter (Millipore). 1.5 mL of TransIT Pro (Mirus Bio LLC) transfection reagent is added and allowed to incubate for 15-30 minutes at room temperature. The complex is then added to the prepared CHO-E cells, and the shake flask returned to the shaker. 24 hours post transfection, 10 mL of Anti-Clumping Agent and 150 mL of CHO CD Efficient Feed B (both from Gibco) are added to the transfected cells and the temperature is shifted to 32° C. The transfected culture is maintained for 6-12 days and monitored routinely throughout the culture for cell growth, viability and nutrient consumption. Culture harvest is completed by centrifugation at 4700 rpm at 4° C., followed by sterile filtration.

BsAbs Purification. Load harvested culture supernatant onto 1 ml HiTrap MabSelect SuRe column from GE (Cat #11003493) pre-equilibrated with buffer A (DPBS, pH7.2) at 1.0 ml/min. Wash the columns with 10 ml each of buffer A, buffer B (DPBS plus 1.0 M NaCl) and buffer A again at 1 ml/min. Then, elute the bound proteins with 30 mM Sodium Acetate, pH3.5. Neutralize 5 ml fraction with 1% volume to volume of 3M Sodium Acetate, PH˜9. The final buffer is 60 mM NaOAc, PH˜5 after Protein A elution. The monomer percentage was 71% for PD1/ISO & 63% for PD1/CD48 by aSEC.

MabSelect Sure purified materials were further polished to remove aggregates by Cation exchange. Poros GoPure HS Pre-packed column from Thermo Fisher (Cat #4481316) was used for ion exchange. Load the Protein A sample onto a 1 ml Poros HS column pre-equilibrated with buffer A (60 mM NaOAc. pH 5.0) and wash the column with 10 column volume of buffer A. Then elute the bound proteins with a gradient from 0% to 40% of buffer B (60 mM NaOA, 1 M NaCl, pH 5.0) in 20 column volume at 0.5 ml/min. Pool fractions around peak and adjust salt concentration to 100 mM NaCl. Sterilely filter the samples with filtration units Measure protein concentration, determine endotoxin level and run SDS-PAGE as well as aSEC.

NFAT Luciferase assay. The Jurkat PD-1 NFAT reporter cell line was generated in-house. Human PD-1 from GeneCopoeia (EX-B0169-M02) was cloned into a vector which was transfected into the Jurkat cells (ATCC) via electroporation. The NFAT luciferase reporter (Promega E8481) was then transfected into PD-1 expressing clones via electroporation. The THP-1 cell line was purchased ATCC (TIB-202) and cultured following manufacturer instructions.

Jurkat PD-1 and NFAT reporter cells were re-suspended in assay media (RPMI, 2% HI-FBS), and 3×10⁴ cells/condition were pre-incubated with doses of BsAbs (starting concentration of 100 nM and 1:3 dilutions) for 15 min in 384 flat-bottom Opti-Plates. THP-1 cells (3×10⁴ cells/condition) were added, and cells were stimulated with a 10 nM solution of aCD3×aCD33 activator for 6 h at 37° C. NFAT reporter was analyzed by addition of Steady-Glo® Luciferase Assay reagent form 15 min (Promega, E2520) and read in EnVision Plate Reader.

### Results

CD48 and PD-1 crosslinking enhance PD-1 phosphorylation. CD48 is a well-established lipid raft- and IS-resident protein in mouse and human lymphocytes (Elishmereni and Levi-Schaffer, 2011). To better qualify the presence and abundance of CD48 in lipid rafts relative to that of PD-1 and CD3 we carried out ImageStream experiments to quantify at the single cell level the co-localization of these receptors with Cholera Toxin (CT)-induced lipid-raft coalescence (capping) in Jurkat cells overexpressing PD-1. Analysis of co-localization of CD48, CD3 and PD-1 within the lipid raft caps induced by CT was carried out using flurophore-labeled MAbs. This analysis showed that, unlike PD-1, CD3 and CD48 were readily observed within the capping induced by CT. Perimeter quantification, in which smaller perimeter values correlate with capping, revealed that after activation the capping of CD48 was evident, yet slightly less abundant than CD3. In contrast, PD-1 normally did not colocalize with CT, consistent with the hypothesis that PD-1 requires an active process (e.g. interaction with PDL-1) in order to be recruited to the lipid-raft enriched IS (Yokosuka et al., 2012).

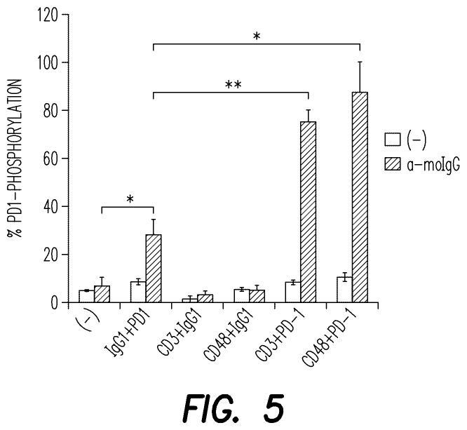

Presence of CD48 into lipid rafts and constitutive association with Src kinases (Lck in T cells), allowed us to hypothesized that, similar to CD3, approximation of CD48 with PD-1 will induce PD-1 activation/phosphorylation, as the canonical mechanism for PD-1 activation requires Lck-mediated phosphorylation of the intracellular ITSM and ITIM domains of PD-1 (Chemnitz et al., 2004; Parry et al., 2005; Sheppard et al., 2004). To test this hypothesis, a customized Jurkat cell line was generated to express human PD-1 fusion protein with one half of b-galactosidase (PK), and a cytosolic full-length SHP1 fusion protein with the complementary half of b-galactosidase (EA). PD-1 activation was thus measured as a function of PK/EA complementation due to recruitment of SHP1 to phosphorylated PD1, which produces a functional b-galactosidase. After confirming expression of PD-1, CD48 and CD3 in these cells, experiments were set up to evaluate the potential of a MAb against CD48 to induce PD-1 activation upon crosslinking (FIG. 5). PD-1 activation was not induced in the absence of PD-1 MAb or a Fc-specific secondary F(ab′)2 antibody. Crosslinking with the secondary antibody induced PD-1 activation by ˜3 fold; however, in the presence of CD48 or CD3 Mabs, PD-1 activation was enhanced by ˜9 fold, indicating that close association of CD48 or CD3 with PD-1 can boost PD-1 activation. The low level of PD-1 activation induced by self-crosslinking was not surprising as studies have demonstrated that a small fraction of Lck is constitutively associated with PD-1 in T cells (Sheppard et al., 2004).

CD48-dependent activation of PD-1 requires Src-kinase activity. To determine whether the enhancement of PD-1 activation/phosphorylation by CD48 is dependent on Src kinase activity, crosslinking experiments were carried out in the presence of a pan-Src kinase inhibitor PP2, or the inactive analog PP3. Src-kinase inhibition abrogated PD-1 activation upon self-crosslinking or co-crosslinking with CD48, indicating that Lck activity is required for PD-1 activation. To further validate this concept, PD-1 activation was evaluated by crosslinking PD-1 with suboptimal amounts of anti-PD-1 in presence of antibodies against CD71, a receptor that does not migrate into lipid rafts or associate with Src-kinases (Schatzlmaier et al., 2015). Crosslinking of PD-1 with CD71 did not result in PD-1 activation, supporting the finding that translocation of PD-1 to an environment rich in activated Src kinases enables PD-1 phosphorylation and activation.

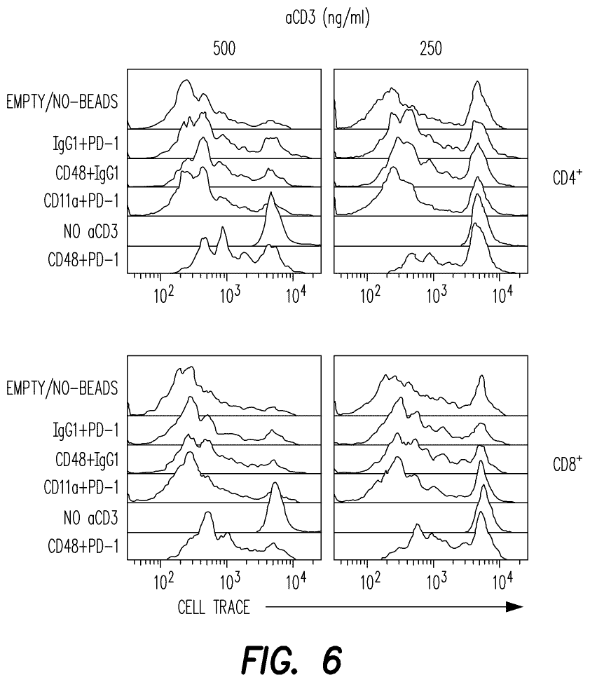

CD48-dependent PD-1 activation blunts AR-induced proliferation of primary human T cells. To functionally evaluate the ability of the CD48-dependent PD-1 activation to modulate T cell functions, magnetic beads were covalently co-coated with CD48, PD-1 MAbs, and isotype control, and tested on primary human T cells (CD4+ and CD8+) stimulated with plate bound anti-CD3. First, we confirmed expression of CD48 and PD-1 in pre-activated human CD4+ and CD8+T lymphocytes. To evaluate the effect of the coated beads on cell activation, human pan-T cell were labeled with CellTrace-Violet, then activated with CD3 MAbs, and cell proliferation was analyzed by dilution of the CellTrace (FIG. 6). As shown in FIG. 6, beads co-coated with both CD48 and PD-1 MAbs were able to significantly reduce T cell proliferation relative to cells treated with beads coated with CD48 or PD-1 MAbs alone, and the inhibitory effect was more significant on CD4⁺than CD8⁺ cells. As an additional control, we also tested beads co-coated with PD-1 and CD11a Mabs; the latter selected on the premise that CD11a is not a constitutively lipid-raft resident protein. As expected, PD-1 had no inhibitory function upon co-recruitment with CD11a. These results further support the hypothesis that PD-1 activation by lipid-raft resident molecules (i.e. CD48) can effectively activate PD-1 to inhibit T cell expansion.

A bispecific antibody against PD-1 and CD48 induces PD-1 activation to modulate cytokine secretion and NFAT activation in AR-stimulated human T cells. The results obtained using separate monoclonal antibody-coated beads, prompted us to generate bispecific antibodies (BsAbs) in order to test the hypothesis that molecular localization of PD-1 with CD48 would provide an inhibitory signal on activated human T cells (FIG. 7A, 7B, 7C). Published antibodies were generated (agonistic anti-PD-1 antibody from patent application WO 2011/110621AI), and anti-CD48 Ab from US2012/0076790), and BsAbs were engineered as knob or hole single heavy/light chain constructs to generate BsAbs containing anti-PD-1 and anti-CD48 (PD-1/CD48), or anti-PD-1 and anti-TNP as control (PD-1/ISO) (FIG. 7A). Binding of each arm to PD-1 or CD48 was demonstrated by flow cytometry on Jurkat cells overexpressing PD-1 in order to detect both PD-1 and CD48 binding, or lacking PD-1 expression, to detect only CD48 binding (FIG. 7B). Using the Jurkat PD-1 complementation assay system described above, we demonstrated that PD-1/CD48 BsAb was ˜3-fold more potent inducing PD-1activation than the PD-1/ISO control, confirming that PD-1/CD48 colocalization using this BsAb format results in enhanced PD-1 phosphorylation as well (FIG. 6). To evaluate the functional effect of the PD-1/CD48 BsAb, human memory CD4+T (PD1⁺) cells were stimulated with plate-bound anti-CD3e in presence of plate-bound PD-1/CD48 BsAbs or control antibodies, and analyzed for cytokine secretion (FIG. 7C). This analysis revealed an immunomodulatory effect of the PD-1/CD48 BsAb as it significantly reduced secretion of the pro-inflammatory cytokine IL2, but enhanced production of the anti-inflammatory cytokine IL-10. As IL-2 secretion requires NFAT transcriptional activation (Chow et al., 1999), the effect of the PD-1/CD48 BsAb on NFAT activation was evaluated in Jurkat T cells expressing both PD-1 and a NFAT-Luciferase reporter, and activated with anti-CD3e in presence of THP-1 cells for co-stimulation. This analysis showed that PD-1/CD48 BsAb was able to reduce the NFAT reporter >10⁻³⁰% than control antibodies, and indicates that CD48-dependent activation of PD-1 is also to inhibit a key T cell effector transcriptional event leading to IL-2 production.

1. An anti-Programmed cell death 1 (PD-1) anti-PD-1 antibody or antigen-binding fragment thereof, wherein comprising:
(i) the anti-PD 1 antibody or antigen-binding fragment thereof comprises:
a heavy chain variable region comprising the amino acid sequence of SEQ ID NO: 73 (H-CDR 1); the amino acid sequence of SEQ ID NO: 76, SEQ ID NO: 78, or SEQ ID NO: 79 (H-CDR2); and the amino acid sequence of SEQ ID NO: 77 (H-CDR 3), and
a light chain variable region comprising the amino acid sequence of SEQ ID NO: 164 or SEQ ID NO: 165 (L-CDR 1); the amino acid sequence of SEQ ID NO: 31, SEQ ID NO: 166, or SEQ ID NO: 167 (L-CDR2); and the amino acid sequence of SEQ ID NO: 32 (L-CDR3); or
(ii) the anti-PD 1 antibody or antigen-binding fragment thereof comprises:
a heavy chain variable region comprising the amino acid sequence of SEQ ID NO: 43 (H-CDR 1); the amino acid sequence of SEQ ID NO: 44 (H-CDR2); and the amino acid sequence of SEQ ID NO: 45 (H-CDR 3), and
a light chain variable region comprising the amino acid sequence of SEQ ID NO: 1 (L-CDR 1); the amino acid sequence of SEQ ID NO: 2 (L-CDR2); and the amino acid sequence of SEQ ID NO: 3 (L-CDR3),
or
a heavy chain variable region comprising the amino acid sequence of SEQ ID NO: 43 (H-CDR 1); the amino acid sequence of SEQ ID NO: 46 (H-CDR2); and the amino acid sequence of SEQ ID NO: 45 (H-CDR3), and
a light chain variable region comprising the amino acid sequence of SEQ ID NO: 1 (L-CDR 1); the amino acid sequence of SEQ ID NO: 2 (L-CDR2); and the amino acid sequence of SEQ ID NO: 3 (L-CDR3),
or
a heavy chain variable region comprising the amino acid sequence of SEQ ID NO: 47 (H-CDR 1); the amino acid sequence of SEQ ID NO: 48 (H-CDR2); and the amino acid sequence of SEQ ID NO: 49 (H-CDR3), and
a light chain variable region comprising the amino acid sequence of SEQ ID NO: 4 (L-CDR 1); the amino acid sequence of SEQ ID NO: 5 (L-CDR2); and the amino acid sequence of SEQ ID NO: 6 (L-CDR3),
or
a heavy chain variable region comprising the amino acid sequence of SEQ ID NO: 50 (H-CDR 1); the amino acid sequence of SEQ ID NO: 51 (H-CDR2); and the amino acid sequence of SEQ ID NO: 52 (H-CDR3), and
a light chain variable region comprising the amino acid sequence of SEQ ID NO: 7 (L-CDR 1); the amino acid sequence of SEQ ID NO: 8 (L-CDR2); and the amino acid sequence of SEQ ID NO: 9 (L-CDR3),
or
a heavy chain variable region comprising the amino acid sequence of SEQ ID NO: 53 (H-CDR 1); the amino acid sequence of SEQ ID NO: 54 (H-CDR2); and the amino acid sequence of SEQ ID NO: 55 (H-CDR3), and
a light chain variable region comprising the amino acid sequence of SEQ ID NO: 10 (L-CDR1); the amino acid sequence of SEQ ID NO: 11 (L-CDR2); and the amino acid sequence of SEQ ID NO: 29 (L-CDR3),
or
a heavy chain variable region comprising the amino acid sequence of SEQ ID NO: 56 (H-CDR 1); the amino acid sequence of SEQ ID NO: 57 (H-CDR2); and the amino acid sequence of SEQ ID NO: 58 (H-CDR3), and
a light chain variable region comprising the amino acid sequence of SEQ ID NO: 13 (L-CDR1); the amino acid sequence of SEQ ID NO: 14 (L-CDR2); and the amino acid sequence of SEQ ID NO: 15 (L-CDR3),
or
a heavy chain variable region comprising the amino acid sequence of SEQ ID NO: 59 (H-CDR1); the amino acid sequence of SEQ ID NO: 60 (H-CDR2); and the amino acid sequence of SEQ ID NO: 61 (H-CDR3), and
a light chain variable region comprising the amino acid sequence of SEQ ID NO: 16 (L-CDR1); the amino acid sequence of SEQ ID NO: 17 (L-CDR2); and the amino acid sequence of SEQ ID NO: 18 (L-CDR3),
or
a heavy chain variable region comprising the amino acid sequence of SEQ ID NO: 62 (H-CDR 1); the amino acid sequence of SEQ ID NO: 63 (H-CDR2); and the amino acid sequence of SEQ ID NO: 64 (H-CDR3), and
a light chain variable region comprising the amino acid sequence of SEQ ID NO: 19 (L-CDR 1); the amino acid sequence of SEQ ID NO: 20 (L-CDR2); and the amino acid sequence of SEQ ID NO: 21 (L-CDR3),
or
a heavy chain variable region comprising the amino acid sequence of SEQ ID NO: 65 (H-CDR 1); the amino acid sequence of SEQ ID NO: 66 (H-CDR2); and the amino acid sequence of SEQ ID NO: 67 (H-CDR3), and
a light chain variable region comprising the amino acid sequence of SEQ ID NO: 22 (L-CDR1); the amino acid sequence of SEQ ID NO: 23 (L-CDR2); and the amino acid sequence of SEQ ID NO: 24 (L-CDR3),
or
a heavy chain variable region comprising the amino acid sequence of SEQ ID NO: 68 (H-CDR 1); the amino acid sequence of SEQ ID NO: 69 (H-CDR2); and the amino acid sequence of SEQ ID NO: 70 (H-CDR3), and
a light chain variable region comprising the amino acid sequence of SEQ ID NO: 25 (L-CDR1); the amino acid sequence of SEQ ID NO: 26 (L-CDR2); and the amino acid sequence of SEQ ID NO: 27 (L-CDR3),
or
a heavy chain variable region comprising the amino acid sequence of SEQ ID NO: 71 (H-CDR 1); the amino acid sequence of SEQ ID NO: 72 (H-CDR2); and the amino acid sequence of SEQ ID NO: 58 (H-CDR3), and
a light chain variable region comprising the amino acid sequence of SEQ ID NO: 28 (L-CDR1); the amino acid sequence of SEQ ID NO: 14 (L-CDR2); and the amino acid sequence of SEQ ID NO: 29 (L-CDR 3),
or
a heavy chain variable region comprising the amino acid sequence of SEQ ID NO: 73 (H-CDR 1); the amino acid sequence of SEQ ID NO: 74 (H-CDR2); and the amino acid sequence of SEQ ID NO: 75 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 29 (L-CDR3),
or
a heavy chain variable region comprising the amino acid sequence of SEQ ID NO: 73 (H-CDR 1); the amino acid sequence of SEQ ID NO: 76 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 78 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR 3),
or
a heavy chain variable region comprising the amino acid sequence of SEQ ID NO: 73 (H-CDR 1); the amino acid sequence of SEQ ID NO: 76 (H-CDR2); and the amino acid sequence of SEQ ID NO: 77 (H-CDR3), and
a light chain variable region comprising the amino acid sequence of SEQ ID NO: 164 (L-CDR1); the amino acid sequence of SEQ ID NO: 31 (L-CDR2); and the amino acid sequence of SEQ ID NO: 29 (L-CDR3),
or
a heavy chain variable region comprising the amino acid sequence of SEQ ID NO: 73 (H-CDR1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 166 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 78 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 166 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 165 (L-CDR 1); the amino acid sequence of SEQ ID NO: 167 (L-CDR2); and the amino acid sequence of SEQ ID NO: 32 (L-CDR3),
or
a heavy chain variable region comprising the amino acid sequence of SEQ ID NO: 80 (H-CDR 1); the amino acid sequence of SEQ ID NO: 81 (H-CDR2); and the amino acid sequence of SEQ ID NO: 82 (H-CDR3), and
a light chain variable region comprising the amino acid sequence of SEQ ID NO: 33 (L-CDR1); the amino acid sequence of SEQ ID NO: 14 (L-CDR2); and the amino acid sequence of SEQ ID NO: 34 (L-CDR3),
or
a heavy chain variable region comprising the amino acid sequence of SEQ ID NO: 83 (H-CDR 1); the amino acid sequence of SEQ ID NO: 84 (H-CDR2); and the amino acid sequence of SEQ ID NO: 85 (H-CDR3), and
a light chain variable region comprising the amino acid sequence of SEQ ID NO: 16 (L-CDR1); the amino acid sequence of SEQ ID NO: 35 (L-CDR2); and the amino acid sequence of SEQ ID NO: 36 (L-CDR3),
or
a heavy chain variable region comprising the amino acid sequence of SEQ ID NO: 86 (H-CDR 1); the amino acid sequence of SEQ ID NO: 87 (H-CDR2); and the amino acid sequence of SEQ ID NO: 88 (H-CDR3), and
a light chain variable region comprising the amino acid sequence of SEQ ID NO: 37 (L-CDR1); the amino acid sequence of SEQ ID NO: 38 (L-CDR2); and the amino acid sequence of SEQ ID NO: 39 (L-CDR3),
or
a heavy chain variable region comprising the amino acid sequence of SEQ ID NO: 89 (H-CDR 1); the amino acid sequence of SEQ ID NO: 90 (H-CDR2); and the amino acid sequence of SEQ ID NO: 91 (H-CDR3), and
a light chain variable region comprising the amino acid sequence of SEQ ID NO: 40 (L-CDR1); the amino acid sequence of SEQ ID NO: 41 (L-CDR2); and the amino acid sequence of SEQ ID NO: 42 (L-CDR 3).

2. (canceled)

3. The anti-PD-1 antibody or antigen-binding fragment thereof of claim 1, wherein said antibody or antigen-binding fragment thereof is a humanized antibody or antigen-binding fragment thereof.

4. The anti-PD-1 antibody or antigen-binding fragment thereof of claim 1, wherein said antibody or antigen-binding fragment thereof is selected from the group consisting of a monoclonal antibody, a Fab, a F(ab′)2, a Fv and an scFv.

5. The anti-PD-1 antibody or antigen-binding fragment thereof of claim 1, wherein;
(i) said antibody or antigen-binding fragment thereof comprises a heavy chain variable region comprising the amino acid sequence of any one of SEQ NO: 131, SEQ NO: 133, SEQ NO: 135, SEQ NO: 137 or SEQ NO: 139 and a light chain variable region comprising the amino acid sequence of any one of SEQ NO: 125, SEQ NO: 127 or SEQ NO: 129; or
(ii) said antibody or antigen-binding fragment thereof comprises a heavy chain variable region and a light chain variable region comprising the amino acid sequences of SEQ ID NO: 108 and SEQ ID NO: 92, respectively; SEQ ID NO: 109 and SEQ ID NO: 93, respectively; SEQ ID NO: 110 and SEQ ID NO: 94, respectively; SEQ ID NO: 111 and SEQ ID NO: 95, respectively; SEQ ID NO: 112 and SEQ ID NO: 96, respectively; SEQ ID NO: 113 and SEQ ID NO: 97, respectively; SEQ ID NO: 114 and SEQ ID NO: 98, respectively; SEQ ID NO: 115 and SEQ ID NO: 99, respectively; SEQ ID NO: 116 and SEQ ID NO: 100, respectively; SEQ ID NO: 117 and SEQ ID NO: 101, respectively; SEQ ID NO: 118 and SEQ ID NO: 102, respectively; SEQ ID NO: 119 and SEQ ID NO: 103, respectively; SEQ ID NO: 120 and SEQ ID NO: 104, respectively; SEQ ID NO: 121 and SEQ ID NO: 105, respectively; SEQ ID NO: 122 and SEQ ID NO: 106, respectively; SEQ ID NO: 123 and SEQ ID NO: 107, respectively; or
(iii) wherein said antibody or antigen-binding fragment thereof comprises:
a heavy chain variable region and a light chain variable region having at least 90% identity to the amino acid sequences of SEQ ID NO: 131 and SEQ ID NO: 125, respectively;
a heavy chain variable region and a light chain variable region having at least 90% identity to the amino acid sequences of SEQ ID NO: 133 and SEQ ID NO: 127, respectively;
a heavy chain variable region and a light chain variable region having at least 90% identity to the amino acid sequences of SEQ ID NO: 135 and SEQ ID NO: 127, respectively;
a heavy chain variable region and a light chain variable region having at least 90% identity to the amino acid sequences of SEQ ID NO: 137 and SEQ ID NO: 129, respectively; or
a heavy chain variable region and a light chain variable region having at least 90% identity to the amino acid sequences of SEQ ID NO: 139 and SEQ ID NO: 129, respectively.

6-8. (canceled)

9. The anti-PD-1 antibody of claim 1, wherein said antibody comprises a heavy chain constant region selected from the group consisting of IgG1, IgG2, IgG3, IgG4, IgM, IgA and IgE constant regions.

10. The anti-PD-1 antibody of claim 9, wherein;
(i) the heavy chain constant region is a heavy chain constant region of a human IgG1 or a mouse IgG1, wherein the heavy chain constant region has a leucine to alanine mutation at a position corresponding to residue 235 of SEQ ID NO: 161 and at a position corresponding to residue 236 of SEQ ID NO: 161; or
(ii) wherein the heavy chain constant region is a heavy chain constant region of a human IgG4 or a mouse IgG 4, wherein the heavy chain constant region has an IgG 4 with a serine to proline Ser228Pro mutation at a position corresponding to residue 226 of SEQ ID NO: 143, SEQ ID NO: 147, SEQ ID NO: 149, SEQ ID NO: 153, or SEQ ID NO: 155; or (ii).

11. The anti-PD-1 antibody of claim 9, wherein the heavy chain constant region is a heavy chain constant region of an IgG1 or IgG4.

12. (canceled)

13. The anti-PD-1 antibody or antigen-binding fragment thereof of claim 1, wherein the antibody or antigen-binding fragment thereof comprises a light chain constant region selected from the group consisting of kappa and lambda.

14. The anti-PD-1 antibody of claim 1, wherein said antibody comprises:
a heavy chain and a light chain comprising the amino acid sequences of SEQ ID NO: 143 and SEQ ID NO: 141, respectively;
a heavy chain and a light chain comprising the amino acid sequences of SEQ ID NO: 147 and SEQ ID NO: 145, respectively;
a heavy chain and a light chain comprising the amino acid sequences of SEQ ID NO: 149 and SEQ ID NO: 145, respectively;
a heavy chain and a light chain comprising the amino acid sequences of SEQ ID NO: 153 and SEQ ID NO: 151, respectively; or
a heavy chain and a light chain comprising the amino acid sequences of SEQ ID NO: 155 and SEQ ID NO: 151, respectively.

15. (canceled)

16. A pharmaceutical composition comprising an anti-PD-1 antibody or antigen-binding fragment thereof of claim 1, and a pharmaceutically acceptable excipient.

17-19. (canceled)

20. An isolated polynucleotide encoding a heavy chain variable region and/or a light chain variable region of claim 5.

21. (canceled)

22. An expression vector comprising a polynucleotide according to claim 20.

23. An isolated host cell comprising an expression vector according to claim 22.

24. The isolated host cell of claim 23, wherein the cell is a mammalian cell.

25-27. (canceled)

28. A method of manufacturing an antibody comprising the steps of:
culturing an isolated host cell comprising an expression vector comprising an isolated polynucleotide encoding a heavy chain variable region according to claim 5 and an expression vector comprising an isolated polynucleotide encoding a light chain variable region according to claim 5 under conditions that allow formation of an antibody; and
recovering said antibody.

29. The method of claim 28, further comprising the step of purifying said antibody.

30. The method of claim 28, further comprising the step of formulating said antibody into a pharmaceutical composition.

31-39. (canceled)

40. A method of treating a Programmed cell death 1 (PD-1) pathway disorder comprising administering to a patient in need thereof a pharmaceutically effective amount of an anti-PD-1 antibody or antigen-binding fragment thereof, wherein:
(i) the anti-PD 1 antibody or antigen-binding fragment thereof comprises:
a heavy chain variable region comprising the amino acid sequence of SEQ ID NO: 73 (H-CDR 1); the amino acid sequence of SEQ ID NO: 76, SEQ ID NO: 78, or SEQ ID NO: 79 (H-CDR2); and the amino acid sequence of SEQ ID NO: 77 (H-CDR3), and
a light chain variable region comprising the amino acid sequence of SEQ ID NO: 164 or SEQ ID NO: 165 (L-CDR1); the amino acid sequence of SEQ ID NO: 31, SEQ ID NO: 166, or SEQ ID NO: 167 (L-CDR2); and the amino acid sequence of SEQ ID NO: 32 (L-CDR3); or
(ii) the anti-PD 1 antibody or antigen-binding fragment thereof comprises:
a heavy chain variable region comprising the amino acid sequence of SEQ ID NO: 43 (H-CDR 1); the amino acid sequence of SEQ ID NO: 44 (H-CDR2); and the amino acid sequence of SEQ ID NO: 45 (H-CDR3), and
a light chain variable region comprising the amino acid sequence of SEQ ID NO: 1 (L-CDR 1); the amino acid sequence of SEQ ID NO: 2 (L-CDR2); and the amino acid sequence of SEQ ID NO: 3 (L-CDR3),
or
a heavy chain variable region comprising the amino acid sequence of SEQ ID NO: 43 (H-CDR 1); the amino acid sequence of SEQ ID NO: 46 (H-CDR2); and the amino acid sequence of SEQ ID NO: 45 (H-CDR3), and
a light chain variable region comprising the amino acid sequence of SEQ ID NO: 1 (L-CDR 1); the amino acid sequence of SEQ ID NO: 2 (L-CDR2); and the amino acid sequence of SEQ ID NO: 3 (L-CDR3),
or
a heavy chain variable region comprising the amino acid sequence of SEQ ID NO: 47 (H-CDR 1); the amino acid sequence of SEQ ID NO: 48 (H-CDR2); and the amino acid sequence of SEQ ID NO: 49 (H-CDR3), and
a light chain variable region comprising the amino acid sequence of SEQ ID NO: 4 (L-CDR 1); the amino acid sequence of SEQ ID NO: 5 (L-CDR2); and the amino acid sequence of SEQ ID NO: 6 (L-CDR3),
or
a heavy chain variable region comprising the amino acid sequence of SEQ ID NO: 50 (H-CDR 1); the amino acid sequence of SEQ ID NO: 51 (H-CDR2); and the amino acid sequence of SEQ ID NO: 52 (H-CDR3), and
a light chain variable region comprising the amino acid sequence of SEQ ID NO: 7 (L-CDR1); the amino acid sequence of SEQ ID NO: 8 (L-CDR2); and the amino acid sequence of SEQ ID NO: 9 (L-CDR3),
or
a heavy chain variable region comprising the amino acid sequence of SEQ ID NO: 53 (H-CDR 1); the amino acid sequence of SEQ ID NO: 54 (H-CDR2); and the amino acid sequence of SEQ ID NO: 55 (H-CDR3), and
a light chain variable region comprising the amino acid sequence of SEQ ID NO: 10 (L-CDR1); the amino acid sequence of SEQ ID NO: 11 (L-CDR2); and the amino acid sequence of SEQ ID NO: 12 (L-CDR3),
or
a heavy chain variable region comprising the amino acid sequence of SEQ ID NO: 56 (H-CDR 1); the amino acid sequence of SEQ ID NO: 57 (H-CDR2); and the amino acid sequence of SEQ ID NO: 58 (H-CDR3), and
a light chain variable region comprising the amino acid sequence of SEQ ID NO: 13 (L-CDR1); the amino acid sequence of SEQ ID NO: 14 (L-CDR2); and the amino acid sequence of SEQ ID NO: 15 (L-CDR3),
or
a heavy chain variable region comprising the amino acid sequence of SEQ ID NO: 59 (H-CDR 1); the amino acid sequence of SEQ ID NO: 60 (H-CDR2); and the amino acid sequence of SEQ ID NO: 61 (H-CDR3), and
a light chain variable region comprising the amino acid sequence of SEQ ID NO: 16 (L-CDR1); the amino acid sequence of SEQ ID NO: 17 (L-CDR2); and the amino acid sequence of SEQ ID NO: 18 (L-CDR3),
or
a heavy chain variable region comprising the amino acid sequence of SEQ ID NO: 62 (H-CDR 1); the amino acid sequence of SEQ ID NO: 63 (H-CDR2); and the amino acid sequence of SEQ ID NO: 64 (H-CDR3), and
a light chain variable region comprising the amino acid sequence of SEQ ID NO: 19 (L-CDR1); the amino acid sequence of SEQ ID NO: 20 (L-CDR2); and the amino acid sequence of SEQ ID NO: 21 (L-CDR3),
or
a heavy chain variable region comprising the amino acid sequence of SEQ ID NO: 65 (H-CDR1); the amino acid sequence of SEQ ID NO: 66 (H-CDR2); and the amino acid sequence of SEQ ID NO: 67 (H-CDR3), and
a light chain variable region comprising the amino acid sequence of SEQ ID NO: 22 (L-CDR1); the amino acid sequence of SEQ ID NO: 23 (L-CDR2); and the amino acid sequence of SEQ ID NO: 24 (L-CDR3),
or
a heavy chain variable region comprising the amino acid sequence of SEQ ID NO: 68 (H-CDR 1); the amino acid sequence of SEQ ID NO: 69 (H-CDR2); and the amino acid sequence of SEQ ID NO: 70 (H-CDR3), and
a light chain variable region comprising the amino acid sequence of SEQ ID NO: 25 (L-CDR1); the amino acid sequence of SEQ ID NO: 26 (L-CDR2); and the amino acid sequence of SEQ ID NO: 27 (L-CDR3),
or
a heavy chain variable region comprising the amino acid sequence of SEQ ID NO: 71 (H-CDR 1); the amino acid sequence of SEQ ID NO: 72 (H-CDR2); and the amino acid sequence of SEQ ID NO: 58 (H-CDR3), and
a light chain variable region comprising the amino acid sequence of SEQ ID NO: 28 (L-CDR1); the amino acid sequence of SEQ ID NO: 14 (L-CDR2); and the amino acid sequence of SEQ ID NO: 29 (L-CDR3),
or
a heavy chain variable region comprising the amino acid sequence of SEQ ID NO: 73 (H-CDR 1); the amino acid sequence of SEQ ID NO: 74 (H-CDR2); and the amino acid sequence of SEQ ID NO: 75 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 76 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 78 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 30 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 76 (H-CDR2); and the amino acid sequence of SEQ ID NO: 77 (H-CDR3), and
a light chain variable region comprising the amino acid sequence of SEQ ID NO: 164 (L-CDR1); the amino acid sequence of SEQ ID NO: 31 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 166 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 78 (H-CDR2); and the amino acid sequence of SEQ ID NO: 77 (H-CDR3), and
a light chain variable region comprising the amino acid sequence of SEQ ID NO: 165 (L-CDR 1); the amino acid sequence of SEQ ID NO: 166 (L-CDR2); and the amino acid sequence of SEQ ID NO: 32 (L-CDR3),
or
a heavy chain variable region comprising the amino acid sequence of SEQ ID NO: 73 (H-CDR 1); the amino acid sequence of SEQ ID NO: 79 (H-CDR2); and the amino acid sequence of SEQ ID NO: 77 (H-CDR3), and
a light chain variable region comprising the amino acid sequence of SEQ ID NO: 165 (L-CDR1); the amino acid sequence of SEQ ID NO: 167 (L-CDR2); and the amino acid sequence of SEQ ID NO: 32 (L-CDR3),
or
a heavy chain variable region comprising the amino acid sequence of SEQ ID NO: 80 (H-CDR 1); the amino acid sequence of SEQ ID NO: 81 (H-CDR2); and the amino acid sequence of SEQ ID NO: 82 (H-CDR3), and
a light chain variable region comprising the amino acid sequence of SEQ ID NO: 33 (L-CDR1); the amino acid sequence of SEQ ID NO: 14 (L-CDR2); and the amino acid sequence of SEQ ID NO: 34 (L-CDR3),
or
a heavy chain variable region comprising the amino acid sequence of SEQ ID NO: 83 (H-CDR 1); the amino acid sequence of SEQ ID NO: 84 (H-CDR2); and the amino acid sequence of SEQ ID NO: 85 (H-CDR3), and
a light chain variable region comprising the amino acid sequence of SEQ ID NO: 16 (L-CDR1); the amino acid sequence of SEQ ID NO: 35 (L-CDR2); and the amino acid sequence of SEQ ID NO: 36 (L-CDR3),
or
a heavy chain variable region comprising the amino acid sequence of SEQ ID NO: 86 (H-CDR 1); the amino acid sequence of SEQ ID NO: 87 (H-CDR2); and the amino acid sequence of SEQ ID NO: 88 (H-CDR3), and
a light chain variable region comprising the amino acid sequence of SEQ ID NO: 37 (L-CDR1); the amino acid sequence of SEQ ID NO: 38 (L-CDR2); and the amino acid sequence of SEQ ID NO: 29 (L-CDR3),
or
a heavy chain variable region comprising the amino acid sequence of SEQ ID NO: 89 (H-CDR 1); the amino acid sequence of SEQ ID NO: 90 (H-CDR2); and the amino acid sequence of SEQ ID NO: 91 (H-CDR3), and
a light chain variable region comprising the amino acid sequence of SEQ ID NO: 40 (L-CDR1); the amino acid sequence of SEQ ID NO: 41 (L-CDR2); and the amino acid sequence of SEQ ID NO: 42 (L-CDR 3).

41. The method of claim 40, wherein the disorder is selected from the group consisting of arthritis, rheumatoid arthritis, asthma, Chronic obstructive pulmonary disease (COPD), pelvic inflammatory disease, Alzheimer's Disease, Crohn's disease, ulcerative colitis, Peyronie's Disease, coeliac disease, gallbladder disease, Pilonidal disease, peritonitis, vasculitis, surgical adhesions, stroke, Type I Diabetes, Lyme disease, meningoencephalitis, autoimmune uveitis, multiple sclerosis, lupus, Guillain-Barr syndrome, A topic dermatitis, autoimmune hepatitis, fibrosing alveolitis, Grave's disease, IgA nephropathy, idiopathic thrombocytopenia purpura, Meniere's disease, pemphigus, primary biliary cirrhosis, sarcoidosis, scleroderma, Wegener's granulomatosis, pancreatitis, trauma (surgery), graft-versus-host disease, transplant rejection, heart disease, myocardial infarction, atherosclerosis, intravascular coagulation, bone resorption, osteoporosis, osteoarthritis, periodontitis, hypochlorhydria, infertility related to lack of fetal-maternal tolerance, Sjogren's Syndrome, vitiligo, and myasthenia gravis.

42. The method of claim 40, wherein the disorder is selected from the group consisting of systemic sclerosis (SSc), systemic lupus erythematosus, polymyositis, giant cell arteritis, psoriasis, psoriatic arthritis, ankylosing spondylitis, vitiligo, rheumatoid arthritis, and inflammatory bowel disease.

43. The method of claim 40, wherein said antibody or antigen-binding fragment thereof is administered by a parenteral route, intravenous route or subcutaneous route of administration.

44. The method of claim 40, wherein the patient is a human.
